(19) United States
(12) Patent Application Publication
(10) Pub. No.: US 2023/0248816 A9
(43) Pub. Date: Aug. 10, 2023

(54) PROCESS FOR PREPARING A COMPOSITION COMPRISING A PROTEIN D POLYPEPTIDE

(71) Applicant: GLAXOSMITHKLINE BIOLOGICALS SA, Rixensart (BE)

(72) Inventors: Pierre SAE HOUER, Rixensart (BE); Pedro SANTANA DOS SANTOS, Rixensart (BE); Laurent Bernard Jean STRODIOT, Rixensart (BE); Bram VUYLSTEKE, Rixensart (BE); Arnaud ZULIANI, Rixensart (BE)

(73) Assignee: GLAXOSMITHKLINE BIOLOGICALS SA, Rixensart (BE)

(21) Appl. No.: 17/632,270

(22) PCT Filed: Aug. 3, 2020
(86) PCT No.: PCT/EP2020/071761

§ 371 Date: Feb. 2, 2022

(30) Foreign Application Priority Data
Aug. 5, 2019 (EP) 19189964.0

(51) Int. Cl. A61K 39/145; A61K 47/26; A61K 47/10; A61K 39/07; A61K 39/104; A61K 47/02

(52) U.S. Cl. CPC A61K 39/145; A61K 47/26; A61K 47/10; A61K 39/07; A61K 39/1045; A61K 47/02

(57) ABSTRACT

The present invention relates to a process for preparing immunogenic compositions. More particularly, it relates to a process for preparing liquid compositions of Protein D polypeptide and their use in preparing immunogenic compositions comprising Protein D polypeptide which may be used in the treatment or prevention of an acute exacerbation of chronic obstructive pulmonary disease (AECOPD) in a subject, e.g. human.

### TECHNICAL FIELD

The present invention relates to a process for preparing immunogenic compositions. More particularly, it relates to a process for preparing liquid compositions of Protein D polypeptide and their use in preparing immunogenic compositions comprising Protein D polypeptide which may be used in the treatment or prevention of an acute exacerbation of chronic obstructive pulmonary disease (AECOPD) in a subject, e.g. human.

### BACKGROUND TO THE INVENTION

Chronic Obstructive Pulmonary Disease (COPD) is a chronic inflammatory disorder resulting in irreversible decline in lung function as a consequence of inhalation of tobacco smoke or other irritants. Chronic obstructive pulmonary disease (COPD) is recognised as encompassing several conditions (airflow obstruction, chronic bronchitis, bronchiolitis or small airways disease and emphysema) that often coexist (Wilson et al., Eur. Respir. J. 2001; 17: 995-1007). Patients suffer exacerbations of their condition that are usually associated with increased breathlessness, and often have increased cough that may be productive of mucus or purulent sputum (Wilson, Eur Respir J 2001 17:995-1007). COPD is defined physiologically by the presence of irreversible or partially reversible airway obstruction in patients with chronic bronchitis and/or emphysema (Standards for the diagnosis and care of patients with chronic obstructive pulmonary disease. American Thoracic Society. Am J Respir Crit Care Med. 1995 November; 152(5 Pt 2):S77-121).

COPD is a major cause of morbidity and mortality worldwide. Approximately one in 20 deaths in 2005 in the US had COPD as the underlying cause (Drugs and Aging 26:985-999 (2009)). It is projected that in 2020 COPD will rise to the fifth leading cause of disability adjusted life years, chronic invalidating diseases, and to the third most important cause of mortality (Lancet 349:1498-1504 (1997)). The course of COPD is characterized by progressive worsening of airflow limitation and a decline in pulmonary function. COPD may be complicated by frequent and recurrent acute exacerbations (AE), which are associated with enormous health care expenditure and high morbidity (Proceedings of the American Thoracic Society 4:554-564 (2007)). One study suggests that approximately 50% of acute exacerbations of symptoms in COPD are caused by non-typeable Haemophilus influenzae, Moraxella catarrhalis, Streptococcus pneumoniae, and Pseudomonas aeruginosa. (Drugs and Aging 26:985-999 (2009)). Haemophilus influenzae (H. influenzae) is found in 20-30% of exacerbations of COPD; Streptococcus pneumoniae, in 10-15% of exacerbations of COPD; and Moraxella catarrhalis, in 10-15% of exacerbations of COPD (New England Journal of Medicine 359:2355-2365 (2008)). Haemophilus influenzae, Streptococcus pneumoniae, and Moraxella catarrhalis have been shown to be the primary pathogens in acute exacerbations of bronchitis in Hong Kong, South Korea, and the Philippines, while Klebsiella spp., Pseudomonas aeruginosa and Acinetobacter spp. constitute a large proportion of pathogens in other Asian countries/regions including Indonesia, Thailand, Malaysia and Taiwan (Respirology, (2011) 16, 532-539; doi:10.1111/j.1440.1843.2011.01943.x). In Bangladesh, 20% of patients with COPD showed positive sputum culture for Pseudomonas, Klebsiella, Streptococcus pneumoniae and Haemophilus influenzae, while 65% of patients with AECOPD (acute exacerbation of COPD) showed positive cultures for Pseudomonas, Klebsiella, Acinetobacter, Enterobacter, Moraxella catarrhalis and combinations thereof. (Mymensingh Medical Journal 19:576-585 (2010)). However, it has been suggested that the two most important measures to prevent COPD exacerbation are active immunizations and chronic maintenance of pharmacotherapy (Proceedings of the American Thoracic Society 4:554-564 (2007)).

One of the difficulties in treating and managing COPD is the heterogeneity of this complex disease in terms of severity, progression, exercise tolerance, and nature of symptoms. This complexity is also evident in acute exacerbations of COPD (AECOPD), which are transient and apparently stochastic periods of increased COPD symptoms requiring additional medical treatment and often hospitalization (Sethi et al., N Eng J Med 2008; 359:2355-85). Known subtypes of exacerbations are defined by the nature of key triggers including bacterial or viral infections, and/or high eosinophil levels, and these events are typically treated with a combination of antibiotics and steroids in a non-specific manner (Bafadhel et al., Am J Respir Crit Care Med 2011; 184:662). A Protein D polypeptide from Haemophilus influenzae together with a PE-PilA fusion protein and an UspA2 polypeptide from Moraxella catarrhalis is proposed as a vaccine in the treatment or prevention of acute exacerbations of COPD (AECOPD), as described in WO2015125118A1.

There exists a need for improved processes for preparing immunogenic compositions. In particular, there is a need for improved processes for preparing immunogenic compositions to help maintain the structure and function of protein antigens. Such considerations include, but are not limited to, chemical stability of the immunogenic composition (e.g. proteolysis or fragmentation of proteins), physical/thermal stability of the immunogenic composition (e.g., aggregation, precipitation, adsorption), compatibility of the immunogenic composition with the container/closure system, interactions between immunogenic composition and inactive ingredients (e.g. buffers, salts, excipients, cryoprotectants), the manufacturing process, the dosage form (e.g., lyophilized, liquid), the environmental conditions encountered during shipping, storage and handling (e.g., temperature, humidity, shear forces), and the length of time between manufacture and usage.

One particular issue is the formation of visible particles in liquid compositions. The presence of particles is dependent on the manufacturing process and manufacturing environment (design, qualification, validation, execution) as well as post-production handling, storage conditions, transportation, and handling by end users. This includes the choice and processing of primary packaging components, and also the design and stability of the formulation, particularly for biotechnology products. Regulatory monographs in Europe and the United States require drug products for parenteral administration to be “practically free” or “essentially free” of visible particles, respectively (Serge Mathonet et al. PDA J Pharm Sci and Tech 2016, 70: 392-408).

The present invention addresses a need for an improved process for preparing liquid compositions of Protein D polypeptide useful in the preparation of immunogenic compositions. According to the present invention, the appearance of visible particles in liquid compositions of Protein D polypeptide has been identified and an improved process and a liquid composition comprising Protein D polypeptide of improved stability is provided.

### SUMMARY OF THE INVENTION

According to the present invention, it has been found the Protein D polypeptides are susceptible to the formation of visible particles, in particular when the Protein D polypeptide is held in a liquid composition. For example, Protein D polypeptide may be held in a liquid composition (as an intermediate storage step, for example whilst the content of the Protein D polypeptide in the liquid composition is measured) prior to mixing the liquid composition comprising the Protein D polypeptide with other antigens. It was not previously known that Protein D polypeptides were susceptible to aggregation and thus the observation of visible particles was surprising. The present invention provides a process which reduces the formation of visible particles of Protein D polypeptide and thus helps to maintain the structure and function of the protein antigen in immunogenic compositions. The process of the present invention comprises diluting the Protein D polypeptide with solution(s) comprising sucrose and poloxamer (e.g. poloxamer 188). According to the present invention it has been found that adding sucrose and poloxamer to liquid Protein D polypeptide compositions reduces particle formation while stabilizing the structure of the Protein D polypeptide.

Accordingly, the present invention provides a process for preparing a liquid composition comprising a Protein D polypeptide (optionally a Protein D polypeptide of SEQ ID NO: 2), wherein the process comprises mixing the Protein D polypeptide with sucrose and poloxamer.

The present invention also provides a liquid composition comprising a Protein D polypeptide, sucrose and poloxamer.

The present invention also provides an immunogenic composition wherein the Protein D polypeptide has been prepared using a process of the invention.

The present invention also provides an immunogenic composition of the invention, for use in the treatment or prevention of an acute exacerbation of COPD (AECOPD) in a subject, e.g. human.

The present invention also provides the use of an immunogenic composition of the invention, in the manufacture of a medicament for the treatment or prevention of an acute exacerbation of COPD (AECOPD) in a subject, e.g. human.

The present invention also provides a method of treatment of an acute exacerbation of COPD (AECOPD) in a subject, e.g. human, at risk of developing an acute exacerbation of COPD (AECOPD), said method comprising administering to said subject, an effective amount of an immunogenic composition of the invention.

The present invention also provides a method of prevention of an acute exacerbation of COPD (AECOPD) in a subject, e.g. human, at risk of developing an acute exacerbation of COPD (AECOPD), said method comprising administering to said subject, an effective amount of an immunogenic composition of the invention.

### DETAILED DESCRIPTION

### Definitions

As used herein, “adjuvant” means a compound or substance that, when administered to a subject in conjunction with a vaccine, immunotherapeutic, or other antigen- or immunogen-containing composition, increases or enhances the subject's immune response to the administered antigen or immunogen (as compared to the immune response that would be obtained in the absence of adjuvant).

As used herein, the term “immunogenic fragment” is a portion of an antigen smaller than the whole, that is capable of eliciting a humoral and/or cellular immune response in a host animal, e.g. human, specific for that fragment. Thus, for example a fragment of a genomic sequence does not include the genomic sequence itself and a fragment of a protein does not include the full length protein sequence itself. Fragments of a protein can be produced using techniques known in the art, e.g. recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid which encodes the polypeptide. An immunogenic fragment of the invention may be derived from an amino acid sequence at least 70%, 80%, 85%, 90%, 91%, 92%, 93%, 94%, 95%, 96%, 97%, 98%, 99%, or 100% identical to a reference sequence (e.g. SEQ ID NO: 1 to 58 of the present invention) which has been modified by the deletion and/or addition and/or substitution of one or more amino acids (e.g. 1, 2, 3, 4, 5, 6, 7, 8, 9, 10, 11 or 12 amino acids). Amino acid substitution may be conservative or non-conservative. In one aspect, amino acid substitution is conservative. Substitutions, deletions, additions or any combination thereof may be combined in a single variant so long as the variant is an immunogenic polypeptide. For an example, an immunogenic fragment may be derived by deletion of the signal peptide.

As used herein, the term “conservative amino acid substitution” involves substitution of a native amino acid residue with a non-native residue such that there is little or no effect on the size, polarity, charge, hydrophobicity, or hydrophilicity of the amino acid residue at that position, and without resulting in decreased immunogenicity. For example, these may be substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Conservative amino acid modifications to the sequence of a polypeptide (and the corresponding modifications to the encoding nucleotides) may produce polypeptides having functional and chemical characteristics similar to those of a reference polypeptide.

As used herein “signal peptide” refers to a short (less than 60 amino acids, for example, 3 to 60 amino acids) polypeptide present on precursor proteins (typically at the N terminus), and which is typically absent from the mature protein. The signal peptide (sp) is typically rich in hydrophobic amino acids. The signal peptide directs the transport and/or secretion of the translated protein through the membrane. Signal peptides may also be called targeting signals, transit peptides, localization signals, or signal sequences. For example, the signal sequence may be a co-translational or post-translational signal peptide.

As used herein a “subject” is a mammal, including humans, non-human primates, and non-primate mammals such as members of the rodent genus (including but not limited to mice and rats) and members of the order Lagomorpha (including but not limited to rabbits). In particular embodiments, the subject is a human.

As further described below, an acute exacerbation of COPD (AECOPD) is an acute event characterised by a worsening of the patient's respiratory symptoms that is beyond normal day-to-day variations. Typically an AECOPD leads to a change in medication.

As used herein, the term “treatment of an acute exacerbation of COPD (AECOPD)” means ameliorating, stabilising, reducing or eliminating the increased symptoms that are a feature of an acute exacerbation in a subject, e.g. human.

As used herein, the phrase “prevention of an acute exacerbation of COPD (AECOPD)” means preventing, reducing the incidence or frequency, or reducing the severity (e.g. airflow obstruction, chronic bronchitis, bronchiolitis or small airways disease and emphysema) of future acute exacerbations in a subject, e.g. human.

As used herein, the term “treatment of a disease caused by H. influenzae and/or M. catarrhalis” means ameliorating, stabilising, reducing or eliminating the increased symptoms that are a feature of a bacterial infection caused by H. influenzae and/or M. catarrhalis in a subject, e.g. human.

As used herein, the phrase “prevention of a disease caused by H. influenzae and/or M. catarrhalis” means preventing, reducing the incidence or frequency, or reducing the severity of future bacterial infections caused by H. influenzae and/or M. catarrhalis in a subject, e.g. human.

As used herein, the term “bacterial infection” refers to a positive test for a bacterial pathogen on routine culture (Haemophilus influenza or Moraxella catarrhalis) or a total aerobic CFU count greater than or equal to 10⁷ cells. In particular embodiments, the bacterial infection is associated with

a) Haemophilus influenza (e.g. non-typeable H. influenzae (NTHi));

b) Moraxella catarrhalis; or

c) Haemophilus influenzae (e.g. non-typeable H. influenzae (NTHi)) and Moraxella catarrhalis.

As used herein, the term “effective amount” in the context of administering an immunogenic composition or vaccine of the invention to a subject refers to the amount of the immunogenic composition or vaccine which has a prophylactic and/or therapeutic effect.

As used herein “w/v” means weight/volume of the formulation.

Identity between polypeptides may be calculated by various algorithms. In general, when calculating percentage identity the two sequences to be compared are aligned to give a maximum correlation between the sequences. This may include inserting “gaps” in either one or both sequences, to enhance the degree of alignment. For example the Needleman Wunsch algorithm (Needleman and Wunsch 1970, J. Mol. Biol. 48: 443-453) for global alignment, or the Smith Waterman algorithm (Smith and Waterman 1981, J. Mol. Biol. 147: 195-197) for local alignment may be used, e.g. using the default parameters (Smith Waterman uses BLOSUM 62 scoring matrix with a Gap opening penalty of 10 and a Gap extension penalty of 1). A preferred algorithm is described by Dufresne et al. in Nature Biotechnology in 2002 (vol. 20, pp. 1269-71) and is used in the software GenePAST (Genome Quest Life Sciences, Inc. Boston, Mass.). The GenePAST “percent identity” algorithm finds the best fit between the query sequence and the subject sequence, and expresses the alignment as an exact percentage. GenePAST makes no alignment scoring adjustments based on considerations of biological relevance between query and subject sequences. Identity between two sequences is calculated across the entire length of both sequences and is expressed as a percentage of the reference sequence (e.g. SEQ ID NOs. 1 to 58 of the present invention). For fragments, the reference sequence is the longest sequence.

As used herein, the term “particles” refers to “visible particles” and “subvisible particles”. In an embodiment, the particles have an average diameter of 35 to 70 μm.

As used herein, the term “visible particles” refers to insoluble or partially soluble solids in a liquid composition, e.g. an aqueous solution, that are visible to a human eye. In an embodiment, the visible particles have an average diameter of at least 50 μm. In another embodiment, the visible particles have an average diameter of 50-1000 μm. In another embodiment, the visible particles have an average diameter of 75-1000 μm. In another embodiment, the visible particles have an average diameter of 100-1000 μm. In an embodiment, the visible particles are visible when detected by the method described by European Pharmacopeia 5.0, Section 2.9.20. As used herein, “essentially free of visible particles” refers to a liquid composition that does not contain visible particles according to the methods described by European Pharmacopeia 5.0, Section 2.9.20.

As used herein, “sub-visible particles” refers to particulate matter detectable by the Light Obscuration Particle Count Test described in the U.S. Pharmacopoeia, <788>. In an embodiment, the subvisible particles have an average diameter of 2-175 μm. In an embodiment, the subvisible particles have an average diameter of 2-125 μm. In another embodiment, the subvisible particles have an average diameter of less than 50 μm. In another embodiment, the subvisible particles have an average diameter of 2-50 μm.

As used herein, “stable” refers to a composition that, when stored in a container or vial, does not show a significant increase in the number of visible particles over a specified period of time. In an embodiment, the composition, when stored in a container or vial, also does not show a significant increase in the number of subvisible particles over a specified period of time. In some embodiments, the composition is stable for at least 1, 2, 3, 4, 5, 6, 7 or 14 days (i.e. the specified period of time is at least 1, 2, 3, 4, 5, 6, 7 or 14 days).

### DESCRIPTION OF FIGURES

FIG. 1: Representation of the visual inspections; −, + and ++ were depicted as 0, 5 and 10 respectively.

FIG. 2: Day 1, sum of visible particles from 35 to 70 microns: significant interaction observed between Sucrose and NaCl.

FIG. 3: Day 7 sum of 35 to 70 microns: significant effect of sucrose.

FIG. 4: Day 7 sum of 35 to 70 microns: significant effect of NaCl.

FIG. 5: Day 7, average of visible particles observed: Significant interaction observed between Poloxamer 188 and pH.

FIG. 6: Day 7, average of visible particles observed: significant effect observed for sucrose.

FIG. 7: Flowsheet for Optimized Process: Protein D dilution and filtration flow sheet (1 mg/mi in 150 mM NaCl, 10% w/v Sucrose, 1% w/v Poloxamer 188, Phosphate buffer 12.5 mM PO₄³⁻ KH₂PO₄/K₂HPO₄ pH 6.8).

FIG. 8: Flowsheet for Reference Process.

FIG. 9: Occhio particles counting: represents the sum of particles from 50 to 1000 μm detected by Occhio at 3 time points (1, 7 & 14 days) for the optimized liquid composition & reference samples.

FIG. 10: Example of the pictures of visible particles captured by Occhio on a Protein D reference sample (1 mg/ml in 150 mM NaC).

FIG. 11: represents the multivariate analysis (PCA) considering the entire range of the Light Obscuration and Occhio measurements.

FIG. 12: represents the average scores from the observers having performed the visual inspection in a black & white post on 3 different lots.

FIG. 13: Far UV Circular Dichroism

FIG. 14: Far UV Circular Dichroism Difference Spectrum

### COMPOSITION USED FOR DILUTION OF PROTEIN D POLYPEPTIDE

The present invention provides a process for preparing a liquid composition comprising a Protein D polypeptide. The present invention is based on the use of sucrose and/or poloxamer in the dilution of Protein D polypeptide to mitigate the formation of Protein D polypeptide particles. As described in the Examples, it has been surprisingly found that the addition of sucrose and/or poloxamer to a liquid composition comprising a Protein D polypeptide reduces the number of visible particles and subvisible particles formed in the liquid composition. The Protein D polypeptide is mixed with solution(s) comprising sucrose and/or poloxamer to form a liquid composition. Thus the present invention provides an improved process for preparing a liquid composition of Protein D polypeptide which reduces particle formation. The present invention also provides a liquid composition of Protein D polypeptide with improved stability. The present invention provides a liquid composition of Protein D polypeptide with improved stability compared to a liquid composition of Protein D polypeptide formulated without sucrose and poloxamer. Optionally, the process comprises mixing the Protein D polypeptide with both sucrose and poloxamer. Thus the process for preparing a liquid composition comprising a Protein D polypeptide (e.g. a Protein D polypeptide of SEQ ID NO: 2), comprises mixing the Protein D polypeptide with sucrose and poloxamer (e.g. poloxamer 188). In an embodiment, the process for preparing a liquid composition comprising a Protein D polypeptide (e.g. a Protein D polypeptide of SEQ ID NO: 2), comprises mixing the Protein D polypeptide with sucrose, poloxamer (e.g. poloxamer 188) and a salt (e.g. NaC). In another embodiment, the process for preparing a liquid composition comprising a Protein D polypeptide (e.g. a Protein D polypeptide of SEQ ID NO: 2), comprises mixing the Protein D polypeptide with sucrose, poloxamer (e.g. poloxamer 188), a salt (e.g. NaC) and a buffer (e.g. phosphate buffer). In another embodiment, the process comprises mixing the Protein D polypeptide with sucrose and poloxamer prior to mixing the Protein D polypeptide with other antigens.

### Protein D

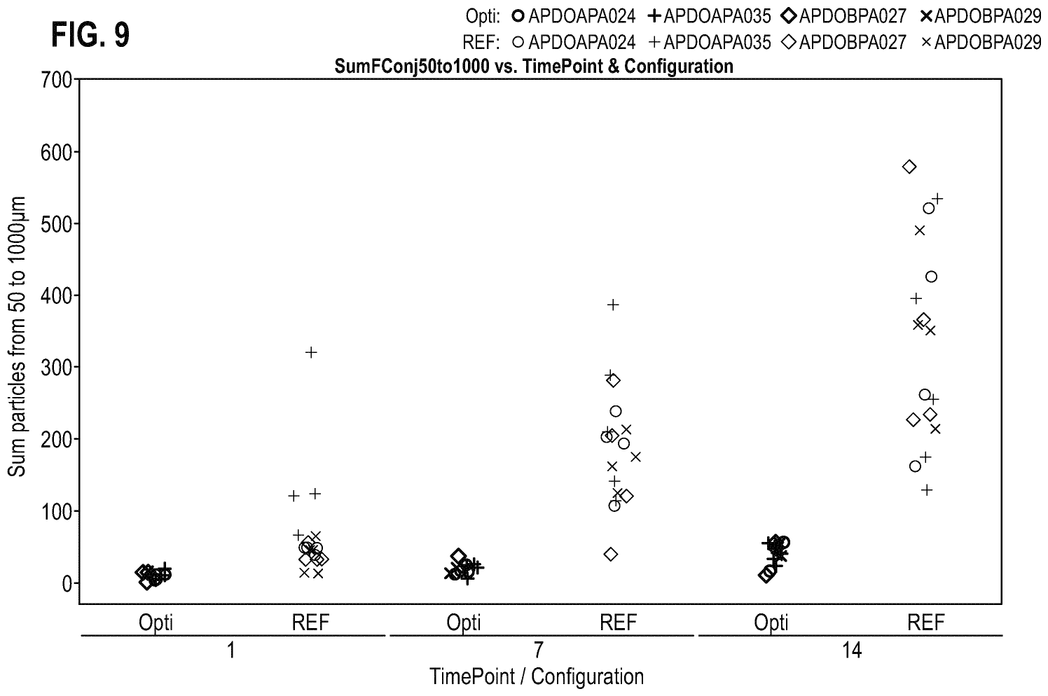

As used herein “Protein D”, “protein D” and “PD” mean Protein D from H. influenzae. Protein D (PD) from Haemophilus influenzae is described in WO91/18926 and EP0594810. Protein D from Haemophilus influenzae may be a Protein D sequence from FIG. 9 (FIG. 9a and 9b together, 364 amino acids) of EP0594610 (SEQ ID NO: 1). Protein D polypeptides may be full length Protein D or an immunogenic fragment thereof (e.g. Protein D polypeptides are described in WO00/56360). For example, the Protein D polypeptide may comprise (or consist) of the Protein D fragment described in EP0594610 beginning at the sequence SSHSSNMANT (SerSerHisSerSerAsnMetAlaAsnThr) (SEQ ID NO: 3), and lacking the 19 N-terminal amino acids from FIG. 9 of EP0594610, optionally with the addition of the tripeptide MDP from NS1 fused to the N-terminal of said Protein D fragment (348 amino acids) (i.e. SEQ ID NO:2). Thus, in an embodiment, the Protein D polypeptide may comprise (or consist) of the amino acid sequence of SEQ ID NO: 2. In an embodiment, the Protein D polypeptide is not conjugated to a polysaccharide, e.g. a polysaccharide from Streptococcus pneumoniae. In an embodiment, the Protein D polypeptide is not conjugated to a polysaccharide from Streptococcus pneumoniae. In an embodiment, the Protein D polypeptide is a free protein (e.g. unconjugated). In an embodiment, the Protein D polypeptide is unlipidated.

SEQ ID NO 1: Protein D (364 amino acids)
MetLysLeuLysThrLeuAlaLeuSerLeuLeuAlaAlaGlyValLeu
AlaGlyCysSerSerHisSerSerAsnMetAlaAsnThrGlnMetLys
SerAspLysIleIleIleAlaHisArgGlyAlaSerGlyTyrLeuPro
GluHisThrLeuGluSerLysAlaLeuAlaPheAlaGlnGlnAlaAsp
TyrLeuGluGlnAspLeuAlaMetThrLysAspGlyArgLeuValVal
IleHisAspHisPheLeuAspGlyLeuThrAspValAlaLysLysPhe
ProHisArgHisArgLysAspGlyArgTyrTyrValIleAspPheThr
LeuLysGluIleGlnSerLeuGluMetThrGluAsnPheGluThrLys
AspGlyLysGlnAlaGlnValTyrProAsnArgPheProLeuTrpLys
SerHisPheArgIleHisThrPheGluAspGluIleGluPheIleGln
GlyLeuGluLysSerThrGlyLysLysValGlyIleTyrProGluIle
LysAlaProTrpPheHisHisGlnAsnGlyLysAspIleAlaAlaGlu
ThrLeuLysValLeuLysLysTyrGlyTyrAspLysLysThrAspMet
ValTyrLeuGlnThrPheAspPheAsnGluLeuLysArgIleLysThr
GluLeuLeuProGlnMetGlyMetAspLeuLysLeuValGlnLeuIle
AlaTyrThrAspTrpLysGluThrGlnGluLysAspProLysGlyTyr
TrpValAsnTyrAsnTyrAspTrpMetPheLysProGlyAlaMetAla
GluValValLysTyrAlaAspGlyValGlyProGlyTrpTyrMetLeu
ValAsnLysGluGluSerLysProAspAsnIleValTyrThrProLeu
ValLysGluLeuAlaGlnTyrAsnValGluValHisProTyrThrVal
ArgLysAspAlaLeuProGluPhePheThrAspValAsnGlnMetTyr
AspAlaLeuLeuAsnLysSerGlyAlaThrGlyValPheThrAspPhe
ProAspThrGlyValGluPheLeuLysGlyIleLys
SEQ ID NO: 2: Protein D fragment with MDP
tripeptide from NS1 (348 amino acids)
MetAspProSerSerHisSerSerAsnMetAlaAsnThrGlnMetLys
SerAspLysIleIleIleAlaHisArgGlyAlaSerGlyTyrLeuPro
GluHisThrLeuGluSerLysAlaLeuAlaPheAlaGlnGlnAlaAsp
TyrLeuGluGlnAspLeuAlaMetThrLysAspGlyArgLeuValVal
IleHisAspHisPheLeuAspGlyLeuThrAspValAlaLysLysPhe
ProHisArgHisArgLysAspGlyArgTyrTyrValIleAspPheThr
LeuLysGluIleGlnSerLeuGluMetThrGluAsnPheGluThrLys
AspGlyLysGlnAlaGlnValTyrProAsnArgPheProLeuTrpLys
SerHisPheArgIleHisThrPheGluAspGluIleGluPheIleGln
GlyLeuGluLysSerThrGlyLysLysValGlyIleTyrProGluIle
LysAlaProTrpPheHisHisGlnAsnGlyLysAspIleAlaAlaGlu
ThrLeuLysValLeuLysLysTyrGlyTyrAspLysLysThrAspMet
ValTyrLeuGlnThrPheAspPheAsnGluLeuLysArgIleLysThr
GluLeuLeuProGlnMetGlyMetAspLeuLysLeuValGlnLeuIle
AlaTyrThrAspTrpLysGluThrGlnGluLysAspProLysGlyTyr
TrpValAsnTyrAsnTyrAspTrpMetPheLysProGlyAlaMetAla
GluValValLysTyrAlaAspGlyValGlyProGlyTrpTyrMetLeu
ValAsnLysGluGluSerLysProAspAsnIleValTyrThrProLeu
ValLysGluLeuAlaGlnTyrAsnValGluValHisProTyrThrVal
ArgLysAspAlaLeuProGluPhePheThrAspValAsnGlnMetTyr
AspAlaLeuLeuAsnLysSerGlyAlaThrGlyValPheThrAspPhe
ProAspThrGlyValGluPheLeuLysGlyIleLys

Thus the Protein D polypeptide sequence for use in the present invention can be modified, for example by truncation of N-terminal or C-terminal residues (e,g, deletion of the N-terminal 19 amino acid residues), by addition of amino acid residues (e.g. the addition of the tripeptide MDP), or by conservative amino acid substitutions. In an embodiment, the Protein D polypeptide has at least 70%, 80%, 85%, 90%, 91%, 92%, 93%, 94%, 95%, 96%, 97%, 98%, 99%, or 100% identity to SEQ ID NO: 1. Immunogenic fragments of Protein D may comprise immunogenic fragments of at least 7, 10, 15, 20, 25, 30 or 50 contiguous amino acids of SEQ ID NO: 1. For example, immunogenic fragments of Protein D may comprise immunogenic fragments of at least 7, 10, 15, 20, 25, 30, 50, 100, 200 or 300 contiguous amino acids of SEQ ID NO: 1, up to 363 contiguous amino acids of SEQ ID NO: 1. The Protein D polypeptide sequence (e.g. SEQ ID NO: 1) may be modified by the deletion and/or addition and/or substitution of one or more amino acids (e.g. 1, 2, 3, 4, 5, 6, 7, 8, 9, 10, 11 or 12 amino acids). The immunogenic fragments may elicit antibodies which can bind SEQ ID NO: 1. In another embodiment, the Protein D polypeptide has at least 70%, 80%, 85%, 90%, 91%, 92%, 93%, 94%, 95%, 96%, 97%, 98%, 99%, or 100% identity to SEQ ID NO: 2. Immunogenic fragments of Protein D may comprise at least 7, 10, 15, 20, 25, 30 or 50 contiguous amino acids of SEQ ID NO: 2. For example, immunogenic fragments of Protein D may comprise immunogenic fragments of at least 7, 10, 15, 20, 25, 30, 50, 100, 200 or 300 contiguous amino acids of SEQ ID NO: 2, up to 347 continuous amino acids of SEQ ID NO: 2. Immunogenic fragments of Protein D may comprise 100, 200, 300, 310, 320, 330 or 340 contiguous amino acids of SEQ ID NO: 2. The Protein D polypeptide sequence (e.g. SEQ ID NO: 2) may be modified by the deletion and/or addition and/or substitution of one or more amino acids (e.g. 1, 2, 3, 4, 5, 6, 7, 8, 9, 10, 11 or 12 amino acids). The immunogenic fragments may elicit antibodies which can bind SEQ ID NO: 2.

In an embodiment, the process comprises mixing the Protein D polypeptide to a concentration of 0.025 to 20 mg/ml, 0.5 to 10 mg/ml, or 0.5 to img/ml Protein D polypeptide in the liquid composition. Specifically, the concentration of Protein D polypeptide may be 0.5 mg/ml or 1 mg/ml. To reach these target concentrations, the Protein D polypeptide content may be analysed by a suitable technique, e.g. RP-UPLC, and diluted accordingly.

### Sucrose

The present invention is based, in part, on the use of sucrose in liquid formulations of Protein D polypeptides to reduce particle formation. In an embodiment, the process comprises mixing the Protein D polypeptide with sucrose to a concentration of 5 to 20% (w/v), 10 to 20% (w/v), or 10 to 15% (w/v) sucrose. Specifically, the concentration of sucrose may be 5%, 10%, 15% or 20% (w/v). To reach these target concentrations, a sucrose solution of higher concentration should be used in the dilution process. For example, to reach the concentration of 10% (w/v) sucrose, a solution of 15.75% (w/v) sucrose may be mixed with the Protein D polypeptide, but it will be understood to the skilled person that variations are possible. In an embodiment, the present invention provides a process for preparing a liquid composition comprising a Protein D polypeptide (e.g. a Protein D polypeptide of SEQ ID NO: 2), wherein the process comprises mixing the Protein D polypeptide with a solution comprising sucrose. In another embodiment, the process comprises mixing the Protein D polypeptide with a solution comprising sucrose, for example to a concentration of 5 to 20% (w/v), 10 to 20% (w/v), or 10 to 15% (w/v).

### Poloxamer

The present invention is based, in part, on the use of poloxamer in liquid formulations of Protein D polypeptides to reduce particle formation. Poloxamers are nonionic triblock linear copolymers composed of a central hydrophobic chain of polyoxypropylene (poly(propylene oxide)) flanked by two hydrophilic chains of polyoxyethylene (poly(ethylene oxide). The length of the polymer can vary. The poloxamer may have a molecular weight in the range of 7,500 to 15,000 or 7,500 to 10,000. Suitably, the poloxamer is selected from the group consisting of poloxamer 124, poloxamer 188, poloxamer 237, poloxamer 338 and poloxamer 407. In an embodiment, the poloxamer is poloxamer 188 (PX188).

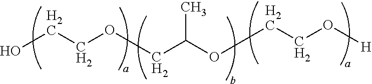

Poloxamer 188 has a molecular weight ranging from 7680 to 9510 Da. Khan et al. (European Journal of Pharmaceutics and Biopharmaceutics, 97 (2015) 60-67) describes generally the use of non-ionic surfactants in therapeutic formulations.

In an embodiment, the process comprises mixing the Protein D polypeptide with poloxamer to a concentration of 0.1 to 1% (w/v), or 0.5 to 1% (w/v) poloxamer. Specifically, the concentration of poloxamer may be 0.5% or 1% (w/v). To reach these target concentrations, a poloxamer solution of higher concentration should be used in the dilution process. For example, to reach the concentration of 1% (w/v) poloxamer (e.g. poloxamer 188), a solution of 10% (w/v) poloxamer (e.g. poloxamer 188) may be mixed with the Protein D polypeptide, but it will be understood to the skilled person that variations are possible.

In an embodiment, the present invention provides a process for preparing a liquid composition comprising a Protein D polypeptide (e.g. a Protein D polypeptide of SEQ ID NO: 2), wherein the process comprises mixing the Protein D polypeptide with a solution comprising poloxamer, for example to a concentration of 0.1 to 1% (w/v), 0.5 to 1% (w/v), or 1% (w/v). In another embodiment, the present invention provides a process for preparing a liquid composition comprising a Protein D polypeptide (e.g. a Protein D polypeptide of SEQ ID NO: 2), wherein the process comprises mixing the Protein D polypeptide with solution(s) comprising sucrose and poloxamer. In another embodiment, the process comprises mixing the Protein D polypeptide with solution(s) comprising: (a) sucrose, for example to a concentration of 5 to 20% (w/v), 10 to 20% (w/v), or 10 to 15% (w/v), and (b) poloxamer (e.g. poloxamer 188) for example to a concentration of 0.1 to 1% (w/v), 0.5 to 1% (w/v), or 1% (w/v). In another embodiment, the present invention provides a process for preparing a liquid composition comprising a Protein D polypeptide, wherein the process comprises mixing the Protein D polypeptide with a solution comprising: (a) sucrose to a concentration of 5 to 20% (w/v), 10 to 20% (w/v), or 10 to 15% (w/v), and (b) poloxamer (optionally poloxamer 188) to a concentration of 0.1 to 1% (w/v), 0.5 to 1% (w/v), or 1% (w/v).

### Salt

As described in the Examples, it has been found that the addition of salt to the liquid composition comprising a Protein D polypeptide also reduces the number of particles formed in the liquid composition (based on the sum of 35 to 70 microns). In an embodiment, the process for preparing a liquid composition comprising a Protein D polypeptide (e.g. a Protein D polypeptide of SEQ ID NO: 2), comprises mixing the Protein D polypeptide with sucrose, poloxamer (e.g. poloxamer 188) and a salt (e.g. NaCl). Thus, in an embodiment the Protein D polypeptide is mixed with sucrose, poloxamer (e.g. poloxamer 188) and a salt (e.g. NaCl). The salt may be for example sodium chloride, calcium chloride, or sodium phosphate. In an embodiment, the immunogenic composition of the invention comprises NaCl (sodium chloride).

The salt (e.g. NaCl) may be added to a concentration of 1 to 200 mM, suitably 10 to 200 mM, 50 to 200 mM, 100 to 200 mM, or 125 to 1755 mM. Specifically, the concentration of salt (e.g. NaCl) may be 150 mM. To reach these target concentrations, a salt (e.g. NaCl) solution of higher concentration should be used in the dilution process. For example, to reach the concentration of 150 mM salt (e.g. NaCl), a solution of 1160 mM salt (e.g. NaCl) may be mixed with the Protein D polypeptide, but it will be understood to the skilled person that variations are possible.

In an embodiment, the present invention provides a process for preparing a liquid composition comprising a Protein D polypeptide (optionally a Protein D polypeptide of SEQ ID NO: 2), wherein the process comprises mixing the Protein D polypeptide with solution(s) comprising (a) sucrose, (b) poloxamer (optionally poloxamer 188) and (c) a salt (optionally NaCl). In another embodiment, the process comprises mixing the Protein D polypeptide with solution(s) comprising: (a) sucrose, for example to a concentration of 5 to 20% (w/v), 10 to 20% (w/v), or 10 to 15% (w/v), (b) poloxamer (e.g. poloxamer 188) for example to a concentration of 0.1 to 1% (w/v), 0.5 to 1% (w/v), or 1% (w/v) and (c) a salt, e.g. NaCl.

### Buffer

In another embodiment, the process for preparing a liquid composition comprising a Protein D polypeptide (e.g. a Protein D polypeptide of SEQ ID NO: 2), comprises mixing the Protein D polypeptide with sucrose, poloxamer (e.g. poloxamer 188), a salt (e.g. NaCl) and a buffer (e.g. phosphate buffer). In an embodiment, said buffer has a pKa of about 3.5 to about 7.5. In some embodiments, the buffer is a phosphate, succinate, histidine or citrate buffer. In certain embodiments, the buffer is a phosphate buffer, suitably potassium phosphate (e.g. KH₂PO₄/K₂HPO₄).

The buffer may be added to a concentration of 5 to 50 mM, suitably 10 to 40 mM, 10 to 30 mM, 10 to 20 mM, or 10 to 15 mM. Specifically, the concentration of buffer may be 10.5 mM, 11.0 mM, 11.5 mM, 12.0 mM, 12.5 mM, 13.0 mM, 13.5 mM, 14.5 mM or 15.0 mM. To reach these target concentrations, a buffer (e.g. phosphate buffer) solution of higher concentration should be used in the dilution process. For example, to reach the concentration of 12.5 mM buffer (e.g. phosphate buffer), a solution of 100 mM buffer (e.g. phosphate buffer) may be mixed with the Protein D polypeptide, but it will be understood to the skilled person that variations are possible.

In an embodiment, the present invention provides a process for preparing a liquid composition comprising a Protein D polypeptide (optionally a Protein D polypeptide of SEQ ID NO: 2), wherein the process comprises mixing the Protein D polypeptide with solution(s) comprising (a) sucrose, (b) poloxamer (optionally poloxamer 188), (c) a salt (optionally NaC) and (d) a buffer (optionally phosphate buffer). In another embodiment, the process comprises mixing the Protein D polypeptide with solution(s) comprising: (a) sucrose, for example to a concentration of 5 to 20% (w/v), 10 to 20% (w/v), or 10 to 15% (w/v), (b) poloxamer (e.g. poloxamer 188) for example to a concentration of 0.1 to 1% (w/v), 0.5 to 1% (w/v), or 1% (w/v) (c) a salt, e.g. NaCl and (d) a buffer (e.g. phosphate buffer).

pH

In an embodiment, the pH of the liquid composition may be adjusted to pH5.5 to 8.5, pH6.0 to 8.0, pH6.4 to 7.7, pH 6.4 to 7.4, pH6.4 to 6.9, pH6.5 to 7.7, pH6.5 to 7.4, pH6.5 to 6.9, pH6.8 to 7.7, pH6.8 to 7.4 or pH6.8 to 6.9. Specifically, the pH of the liquid composition of the invention may be adjusted to pH6.4, pH6.5, pH6.6, pH6.7, pH6.8, pH6.9, pH7.0, pH7.1, pH7.2, pH7.3, pH7.4, pH7.5, pH7.6 or pH7.7. To reach the target pH, a solution of higher pH may be used in the dilution process. It is within the ambit of the skilled person to adjust the pH to reach the target pH. For example, to reach pH6.8, a solution of pH6.9 may be mixed with the liquid composition comprising Protein D polypeptide, but it will be understood to the skilled person that variations are possible.

In an embodiment, the present invention provides a process for preparing a liquid composition comprising a Protein D polypeptide (optionally a Protein D polypeptide of SEQ ID NO: 2), wherein the process comprises mixing the Protein D polypeptide with solution(s) comprising (a) sucrose, (b) poloxamer (optionally poloxamer 188), (c) a salt (optionally NaC), and (d) a buffer (optionally phosphate buffer) to reach a pH 6.4 to 7.7, e.g. pH6.8. In another embodiment, the process comprises mixing the Protein D polypeptide with solution(s) comprising: (a) sucrose, for example to a concentration of 5 to 20% (w/v), 10 to 20% (w/v), or 10 to 15% (w/v), (b) poloxamer (e.g. poloxamer 188) for example to a concentration of 0.1 to 1% (w/v), 0.5 to 1% (w/v), or 1% (w/v), (c) a salt, e.g. NaCl, and (d) a buffer (e.g. phosphate buffer), to reach a pH 6.4 to 7.7, e.g. pH6.8.

### Thawing Protein D Polypeptide

Protein D polypeptide is typically stored in a frozen form (e.g. at −45° C., pH 6.8) and must be thawed prior to formulation. Thawing is the change from a frozen to liquid or semi-liquid state. The process of the invention suitably comprises thawing the Protein D polypeptide. In an embodiment, the process comprises the steps of: (i) thawing the Protein D polypeptide, and (ii) mixing the Protein D polypeptide with sucrose and poloxamer. This forms a liquid composition comprising a Protein D polypeptide. In another embodiment, the process comprises the steps of: (i) thawing the Protein D polypeptide, and (ii) mixing the Protein D polypeptide with sucrose, poloxamer and a salt. In another embodiment, the process comprises the steps of: (i) thawing the Protein D polypeptide, and (ii) mixing the Protein D polypeptide with sucrose, poloxamer, a salt and a buffer. In another embodiment, the process comprises the steps of: (i) thawing the Protein D polypeptide, and (ii) mixing the Protein D polypeptide with: (a) sucrose, for example to a concentration of 5 to 20% (w/v), 10 to 20% (w/v), or 10 to 15% (w/v) and (b) poloxamer (e.g. poloxamer 188) for example to a concentration of to 0.1 to 1% (w/v), 0.5 to 1% (w/v), or 1% (w/v). In another embodiment, step (ii) comprises mixing the Protein D polypeptide with: (a) sucrose, for example to a concentration of 5 to 20% (w/v), 10 to 20% (w/v), or 10 to 15% (w/v), (b) poloxamer (e.g. poloxamer 188) for example to a concentration of 0.1 to 1% (w/v), 0.5 to 1% (w/v), or 1% (w/v) and (c) a salt, e.g. NaCl. In another embodiment, step (ii) comprises mixing the Protein D polypeptide with: (a) sucrose, for example to a concentration of 5 to 20% (w/v), 10 to 20% (w/v), or 10 to 15% (w/v), (b) poloxamer (e.g. poloxamer 188) for example to a concentration of 0.1 to 1% (w/v), 0.5 to 1% (w/v), or 1% (w/v) (c) a salt, e.g. NaCl and (d) a buffer (e.g. phosphate buffer). In an embodiment, step (ii) is carried out to reach a pH 6.4 to 7.7, suitably pH 6.8 (i.e. the pH of the composition following mixing).

Steps (i) and (ii) may occur simultaneously or sequentially. In an embodiment, steps (i) and (ii) occur simultaneously. In another embodiment, steps (i) and (ii) occur sequentially, step (i) followed by step (ii). For example, step (i) may be carried out by raising the temperature of the Protein D polypeptide, e.g. by raising the atmospheric temperature. Suitably, step (i) is carried out statically. Suitably, step (i) is carried out in an incubator. In an embodiment, step (i) is carried out at 1 to 35° C. For example, step (i) may be carried out at 2 to 35° C., 10 to 35° C., 20 to 35° C., 2 to 30° C., 10 to 30° C., 20 to 30° C., 2 to 25° C., or 23 to 27° C. Specifically, step (i) may carried out at room temperature, e.g. 25° C. In an embodiment, step (i) is carried out at 1 to 35° C., for example at 2 to 35° C., or 10 to 35° C., or 15 to 30° C. suitably at room temperature (e.g. 25° C.). In an embodiment, step (i) is carried out at 1 to 35° C. and is followed by step (ii).

Step (i) may also comprise homogenization of the Protein D polypeptide. In an embodiment, step (i) comprises thawing the Protein D polypeptide and homogenizing. For example, the Protein D polypeptide may be homogenized by stirring (e.g. with a magnetic bar) at 100 to 200 RPM, e.g. 150 RPM, suitably for 5 to 10 minutes, e.g. 5 minutes.

Step (ii) comprises mixing the Protein D polypeptide with: (a) sucrose, for example to a concentration of 5 to 20% (w/v), 10 to 20% (w/v), or 10 to 15% (w/v) and (b) poloxamer (e.g. poloxamer 188) for example to a concentration of to 0.1 to 1% (w/v), 0.5 to 1% (w/v), or 1% (w/v). In an embodiment, step (ii) dilutes Protein D polypeptide to the required concentration (as determined by the skilled person) in a liquid composition. In an embodiment, step (ii) comprises stirring, optionally at 2 to 25° C. For example, the process comprises mixing the Protein D polypeptide to a concentration of 0.025 to 20 mg/ml, 0.5 to 10 mg/ml, or 0.5 to 1 mg/ml Protein D polypeptide in the liquid composition. Specifically, the concentration of Protein D polypeptide may be 0.5 mg/ml or 1 mg/ml. The solution(s) comprising the sucrose and poloxamer (and optionally salt and buffer) may be added by pipette or graduated cylinder glass prior to mixing. In an embodiment, individual solutions of sucrose and poloxamer (and optionally salt and buffer) are added separately. In another embodiment, individual solutions of sucrose and poloxamer (and optionally salt and buffer) are added simultaneously. In another embodiment, a single (combined) solution of sucrose and poloxamer (and optionally salt and buffer) is added.

Thus as used herein, the term “solution(s)” means either separate solutions or a single (combined) solution. For example, in a process for preparing a liquid composition comprising a Protein D polypeptide where the process comprises mixing the Protein D polypeptide with solution(s) comprising: (a) sucrose and (b) poloxamer, separate solutions of (a) sucrose and (b) poloxamer may be mixed with the Protein D polypeptide or a single (combined) solution of sucrose and poloxamer may be mixed with the Protein D polypeptide. Suitably, a single (combined) solution of (a) sucrose and (b) poloxamer may be may be mixed with the Protein D polypeptide. For example, in a process for preparing a liquid composition comprising a Protein D polypeptide where the process comprises mixing the Protein D polypeptide with solution(s) comprising: (a) sucrose, (b) poloxamer and (c) salt, separate solutions of (a) sucrose, (b) poloxamer and (c) salt may be may be mixed with the Protein D polypeptide or a single (combined) solution of sucrose, poloxamer and salt may be mixed with the Protein D polypeptide. Suitably, a single (combined) solution of (a) sucrose, (b) poloxamer and (c) salt may be may be mixed with the Protein D polypeptide. For example, in a process for preparing a liquid composition comprising a Protein D polypeptide where the process comprises mixing the Protein D polypeptide with solution(s) comprising: (a) sucrose, (b) poloxamer, (c) salt, and (d) a buffer, separate solutions of (a) sucrose, (b) poloxamer, (c) salt and (d) a buffer may be may be mixed with the Protein D polypeptide or a single (combined) solution of sucrose, poloxamer, salt and a buffer may be mixed with the Protein D polypeptide. Suitably, a single (combined) solution of (a) sucrose, (b) poloxamer, (c) salt and (d) a buffer may be may be mixed with the Protein D polypeptide.

### Filtration

In an embodiment, the process of the present invention comprises filtration of the Protein D polypeptide liquid composition. Accordingly, the present invention provides process for preparing a liquid composition comprising a Protein D polypeptide as described above, subsequently comprising step of filtration, e.g. using a 0.22 μm PVDF membrane. Suitably, the filtration reduces or removes particles of Protein D polypeptide from the liquid composition of Protein D polypeptide. In an embodiment, the present invention provides a process for preparing a liquid composition comprising a Protein D polypeptide comprising step (i) and (ii) and subsequently comprising step of filtration (optionally using a 0.22 μm PVDF membrane) to obtain a liquid composition comprising the Protein D polypeptide in the filtrate. For example the Protein D polypeptide may be filtered by using an OptiScale® 47 filter (0.22 μm Durapore® PVDF membrane 17.7 cm²-Polypropylene cartridge) and a peristaltic pump (flow rate 0.7 ml/min/cm²). Other suitable membranes known to the skilled person may also be used, e.g. PES (polyethersulfone), cellulose. Thus, the present invention provides a process for preparing a liquid composition comprising a Protein D polypeptide which subsequently to the step of mixing the Protein D polypeptide with sucrose and poloxamer comprises the step of filtration (optionally using a 0.22 μm PVDF membrane) to obtain a liquid composition comprising the Protein D polypeptide in the filtrate. Thus, the process of the invention may comprise the steps (in sequential order): (i) thawing the Protein D polypeptide and (ii) mixing the Protein D polypeptide with sucrose and poloxamer followed by the step of filtration.

### Storage

The present invention provides a process for preparing a liquid composition comprising a Protein D polypeptide which reduces the formation of Protein D polypeptide visible particles (and optionally subvisible particles) in particular during storage (a period of time during which the Protein D polypeptide is maintained in a liquid composition). Accordingly, the present invention provides process for preparing a liquid composition comprising a Protein D polypeptide as described above, subsequently comprising the step of storing the liquid composition comprising the Protein D polypeptide. In an embodiment, the present invention provides a process for preparing a liquid composition comprising a Protein D polypeptide comprising steps (i) and (ii) (optionally with filtration) and subsequently comprising the step of storing the liquid composition comprising the Protein D polypeptide. Suitably, the liquid composition comprising a Protein D polypeptide is stored for at least 1 day, at least 7 days, or at least 14 days. In some embodiments, the liquid composition comprising a Protein D polypeptide is stored for at least 1, 2, 3, 4, 5, 6, 7 or 14 days. For example, the liquid composition comprising a Protein D polypeptide may be stored for at least 1 day, suitably up to 7 days (e.g. between 1 to 7 days), or up to 14 days (e.g. between 1 to 14 days). The liquid composition of the invention may be stored at +2 to +8° C. During storage as a liquid composition the content of the Protein D polypeptide in the liquid composition may be measured. Thus, the present invention provides a process for preparing a liquid composition comprising a Protein D polypeptide, which subsequently to the step of mixing the Protein D polypeptide with sucrose and poloxamer (and optionally the step of filtration) comprises the step of storing the liquid composition comprising the Protein D polypeptide. Thus, the process of the invention may comprise the steps (in sequential order): (i) thawing the Protein D polypeptide, (ii) mixing the Protein D polypeptide with sucrose and poloxamer (and optionally the step of filtration) and (iii) storing the liquid composition comprising the Protein D polypeptide.

### Process for Reducing Particle the Formation of Protein D Polypeptide Particles

The present invention provides a process for preparing a liquid composition comprising a Protein D polypeptide which reduces the formation of Protein D polypeptide particles in a liquid composition. In particular, the present invention provides a process for preparing a liquid composition comprising a Protein D polypeptide which reduces the formation of Protein D polypeptide visible particles. In another embodiment, the present invention provides a process for preparing a liquid composition comprising a Protein D polypeptide reduces the formation of Protein D polypeptide visible and subvisible particles. The present invention also provides a method of reducing the formation of Protein D polypeptide particles in a liquid composition, the method comprising a process of the invention. The present invention also provides a process for preparing a liquid composition comprising a Protein D polypeptide which is stable. In an embodiment, the process reduces the formation of Protein D polypeptide visible particles (and optionally subvisible particles) when the liquid composition is stored for at least 1 day. In another embodiment, the process reduces the formation of Protein D polypeptide visible particles (and optionally subvisible particles) when the liquid composition is stored for at least 7 days. In another embodiment, the process reduces the formation of Protein D polypeptide visible particles (and optionally subvisible particles) when the liquid composition is stored for or at least 14 days.

The detection of visible particles in a composition can be determined by any technique deemed suitable by one of ordinary skill in the art. For instance, visible particles may be detected by the method specified in European Pharmacopeia 5.0, section 2.9.20. The detection of subvisible particles in a composition can be determined by any technique deemed suitable by one of ordinary skill in the art. For instance, visible particles may be detected by the Light Obscuration Particle Count Test as described in the U.S. Pharmacopoeia, <788>.

In an embodiment, the process of the present invention reduces the formation of Protein D polypeptide visible particles (and optionally subvisible particles) compared to a process without the addition of sucrose and poloxamer to the Protein D polypeptide composition. In an embodiment, the process of the present invention reduces the formation of Protein D polypeptide visible particles (and optionally subvisible particles) during subsequent storage of the liquid composition comprising the Protein D polypeptide for at least 1, 2, 3, 4, 5, 6, 7 or 14 days compared to a process without the addition of sucrose and poloxamer to the liquid composition comprising Protein D polypeptide. As used herein, “visible particles” refers to insoluble or partially soluble solids in a liquid composition, e.g. an aqueous solution, that are visible to a human eye. In an embodiment, the visible particles have an average diameter of at least 50 μm. In another embodiment, the visible particles have an average diameter of 50-1000 μm. In another embodiment, the visible particles have an average diameter of 75-1000 μm. In another embodiment, the visible particles have an average diameter of 100-1000 μm. In an embodiment, the visible particles are visible when detected by the method described by European Pharmacopeia 5.0, Section 2.9.20. As used herein, “sub-visible particles” refers to particulate matter detectable by the Light Obscuration Particle Count Test described in the U.S. Pharmacopoeia, <788>. In an embodiment, the subvisible particles have an average diameter of 2-175 μm. In an embodiment, the subvisible particles have an average diameter of 2-125 μm. In another embodiment, the subvisible particles have an average diameter of less than 50 μm. In another embodiment, the subvisible particles have an average diameter of 2-50 μm.

Mixing the Liquid Composition Comprising Protein D Polypeptide with Other Antigen(s)

The present invention provides a process for preparing a liquid composition comprising a Protein D polypeptide as described above and subsequently comprising step of mixing the liquid composition comprising the Protein D polypeptide with other antigen(s). In an embodiment, the present invention provides a process for preparing a liquid composition comprising a Protein D polypeptide comprising steps (i), (ii) and (iii) and subsequently comprising step of: (iv) mixing the filtrate comprising the Protein D polypeptide with other antigen(s). In an embodiment, the other antigens comprise Protein E from Haemophilus influenzae or an immunogenic fragment thereof, PilA from Haemophilus influenzae or an immunogenic fragment thereof and a UspA2 polypeptide. In another embodiment, the other antigens comprise a PE-PilA fusion protein and a UspA2 polypeptide. This liquid composition may be used in the preparation of immunogenic compositions. Thus, the present invention provides a process for preparing a liquid composition comprising a Protein D polypeptide, which subsequently to the steps of mixing the Protein D polypeptide with sucrose and poloxamer (and optionally the step of filtration) and the step of storing the liquid composition comprising the Protein D polypeptide comprises the step of mixing the liquid composition comprising the Protein D polypeptide with other antigen(s). Thus, the process of the invention may comprise the steps (in sequential order): (i) thawing the Protein D polypeptide, (ii) mixing the Protein D polypeptide with sucrose and poloxamer (and optionally the step of filtration), (iii) storing the liquid composition comprising the Protein D polypeptide and (iv) mixing the liquid composition comprising the Protein D polypeptide with other antigen(s).

### Protein E

Protein E (PE) is an outer membrane lipoprotein with adhesive properties. It plays a role in the adhesion/invasion of non-typeable Haemophilus influenzae (NTHi) to epithelial cells. (J. Immunology 183: 2593-2601 (2009); The Journal of Infectious Diseases 199:522-531 (2009), Microbes and Infection 10:87-96 (2008)). It is highly conserved in both encapsulated Haemophilus influenzae and non-typeable H. influenzae and has a conserved epithelial binding domain (The Journal of Infectious Diseases 201:414-419 (2010)). Thirteen different point mutations have been described in different Haemophilus species when compared with Haemophilus influenzae Rd as a reference strain. Its expression is observed on both logarithmic growing and stationary phase bacteria. (WO2007/084053). Protein E is also involved in human complement resistance through binding vitronectin. (immunology 183: 2593-2601 (2009)). PE binds vitronectin which is an important inhibitor of the terminal complement pathway. (J. Immunology 183:2593-2601 (2009)).

As used herein “Protein E”, “protein E”, “Prot E”, and “PE” mean Protein E from H. influenzae. Protein E may comprise (or consist) of the amino acid sequence of SEQ ID NO: 4 (corresponding to SEQ ID NO: 4 of WO2012/139225A1): (MKKIILTLSL GLLTACSAQI QKAEQNDVKL APPTDVRSGY IRLVKNVNYY IDSESIWVDN QEPQIVHFDA VVNLDKGLYV YPEPKRYARS VRQYKILNCA NYHLTQVRTD FYDEFWGQGL RAAPKKQKKH TLSLTPDTTL YNAAQIICAN YGEAFSVDKK).

In particular embodiments, the Protein E from Haemophilus influenzae or an immunogenic fragment thereof, suitably has at least 70%, 80%, 85%, 90%, 91%, 92%, 93%, 94%, 95%, 96%, 97%, 98%, 99%, or 100% identity to SEQ ID NO: 4. In an embodiment the Protein E from Haemophilus influenzae is an immunogenic fragment. In another embodiment, the immunogenic fragment of Protein E from Haemophilus influenzae, suitably has at least 70%, 80%, 85%, 90%, 91%, 92%, 93%, 94%, 95%, 96%, 97%, 98%, or 99% identity to SEQ ID NO: 4. For example, immunogenic fragments of Protein E may comprise at least 7, 10, 15, 20, 25, 30 or 50 contiguous amino acids of SEQ ID NO: 4. For example, immunogenic fragments of Protein E may comprise at least 7, 10, 15, 20, 25, 30, 50, 100 or 150 contiguous amino acids of SEQ ID NO: 4, up to 159 contiguous amino acids of SEQ ID NO: 4. The immunogenic fragments may elicit antibodies which can bind SEQ ID NO: 4.

In another embodiment, the Protein E from Haemophilus influenzae or an immunogenic fragment thereof has at least 70%, 80%, 85%, 90%, 91%, 92%, 93%, 94%, 95%, 96%, 97%, 98%, 99%, or 100% identity to SEQ ID NO: 5 (corresponding to SEQ ID NO: 125 of WO2012/139225A1):

SEQ ID NO: 5: Amino acids 20-160 of Protein E
I QKAEQNDVKL APPTDVRSGY IRLVKNVNYY IDSESIWVDN
QEPQIVHFDA VVNLDKGLYV YPEPKRYARS VRQYKILNCA
NYHLTQVRTD FYDEFWGQGL RAAPKKQKKH TLSLTPDTTL
YNAAQIICAN YGEAFSVDKK

In another embodiment, the immunogenic fragment of Protein E from Haemophilus influenzae, suitably has at least 70%, 80%, 85%, 90%, 91%, 92%, 93%, 94%, 95%, 96%, 97%, 98%, 99%, or 100% identity to SEQ ID NO: 5 (corresponding to SEQ ID NO: 125 of WO2012/139225A1). In another embodiment, the immunogenic fragment of Protein E from Haemophilus influenzae, comprises (or consists) of the amino acid sequence of SEQ ID NO: 5 (corresponding to SEQ ID NO: 125 of WO2012/139225A1).

### PilA

Pilin A (PilA) is likely the major pilin subunit of H. influenzae Type IV Pilus (Tfp) involved in twitching motility (Infection and Immunity, 73: 1635-1643 (2005)). NTHi PilA is a conserved adhesin expressed in vivo. It has been shown to be involved in NTHi adherence, colonization and biofilm formation. (Molecular Microbiology 65: 1288-1299 (2007)).

As used herein “PilA” means Pilin A from H. influenzae. PilA may comprise (or consist) of the protein sequence of SEQ ID NO: 6 (corresponding to SEQ ID NO: 58 of WO2012/139225A1) (MKLTTQQTLK KGFTLIELMI VIAIIAILAT IAIPSYQNYT KKAAVSELLQ ASAPYKADVE LCVYSTNETT NCTGGKNGIA ADITTAKGYV KSVTTSNGAI TVKGDGTLAN MEYILQATGN AATGVTWTTT CKGTDASLFP ANFCGSVTQ).

In particular embodiments, the PilA from Haemophilus influenzae or an immunogenic fragment thereof, suitably has at least 70%, 80%, 85%, 90%, 91%, 92%, 93%, 94%, 95%, 96%, 97%, 98%, 99%, or 100% identity to SEQ ID NO: 6. In an embodiment the PilA from Haemophilus influenzae is an immunogenic fragment. In another embodiment, the immunogenic fragment of PilA from Haemophilus influenzae, suitably has at least 70%, 80%, 85%, 90%, 91%, 92%, 93%, 94%, 95%, 96%, 97%, 98%, or 99% identity to SEQ ID NO: 6. For example, immunogenic fragments of PilA may comprise at least 7, 10, 15, 20, 25, 30 or 50 contiguous amino acids of SEQ ID NO: 6. For example, immunogenic fragments of PilA may comprise at least 7, 10, 15, 20, 25, 30, 50 or 100 contiguous amino acids of SEQ ID NO: 6, up to 148 contiguous amino acids of SEQ ID NO: 6. The immunogenic fragments may elicit antibodies which can bind SEQ ID NO: 6.

In another embodiment, the PilA from Haemophilus influenzae or an immunogenic fragment thereof has at least 70%, 80%, 85%, 90%, 91%, 92%, 93%, 94%, 95%, 96%, 97%, 98%, 99%, or 100% identity to SEQ ID NO: 7 (corresponding to SEQ ID NO: 127 of WO2012/139225A1):

SEQ ID NO: 7 Amino acids 40-149 of PilA from H. influenzae strain 86-028NP

T KKAAVSELLQ ASAPYKADVE LCVYSTNETT NCTGGKNGIA ADITTAKGYV KSVTTSNGAI TVKGDGTLAN MEYILQATGN AATGVTWTTT CKGTDASLFP ANFCGSVTQ.

In another embodiment, the immunogenic fragment of PilA, suitably has at least 70%, 80%, 85%, 90%, 91%, 92%, 93%, 94%, 95%, 96%, 97%, 98%, 99%, or 100% identity to SEQ ID NO: 7 (corresponding to SEQ ID NO: 127 of WO2012/139225A1). In another embodiment, the immunogenic fragment of PilA from Haemophilus influenzae, comprises (or consists) of the amino acid sequence of SEQ ID NO: 7 (corresponding to SEQ ID NO: 127 of WO2012/139225A1).

### PE-PilA Fusion Protein

Protein E from Haemophilus influenzae or an immunogenic fragment thereof and PilA from Haemophilus influenzae or an immunogenic fragment thereof may be presented as a fusion protein. Thus, Protein E from Haemophilus influenzae or an immunogenic fragment thereof and PilA from Haemophilus influenzae or an immunogenic fragment thereof are presented as a fusion protein. Suitably, the fusion protein may comprise Protein E from Haemophilus influenzae or an immunogenic fragment thereof at the N-terminus and PilA from Haemophilus influenzae or an immunogenic fragment thereof at the C-terminus of the fusion protein (a PE-PilA fusion protein). In particular, the PE-PilA fusion protein may comprise an immunogenic fragment Protein E from Haemophilus influenzae at the N-terminus and an immunogenic fragment PilA from Haemophilus influenzae at the C-terminus. In an embodiment, the PE-PilA fusion protein has at least 70%, 80%, 85%, 90%, 91%, 92%, 93%, 94%, 95%, 96%, 97%, 98%, 99%, or 100% identity to SEQ ID NO: 8 (LVL-735, corresponding to SEQ ID NO: 194 of WO2012/139225A1).

SEQ ID NO: 8: LVL735 (protein): (pelB sp)(ProtE aa
20-160)(GG)(PilA aa40-149):
MKYLLPTAAA GLLLLAAQPA MAIQKAEQND VKLAPPTDVR
SGYIRLVKNV NYYIDSESIW VDNQEPQIVH FDAVVNLDKG
LYVYPEPKRY ARSVRQYKIL NCANYHLTQV RTDFYDEFWG
QGLRAAPKKQ KKHTLSLTPD TTLYNAAQII CANYGEAFSV
DKKGGTKKAA VSELLQASAP YKADVELCVY STNETTNCTG
GKNGIAADIT TAKGYVKSVT TSNGAITVKG DGTLANMEYI
LQATGNAATG VTWTTTCKGT DASLFPANFC GSVTQ

In an embodiment, the PE-PilA fusion protein comprises (or consists) of the amino acid sequence of SEQ ID NO: 8 (LVL-735 corresponding to SEQ ID NO: 194 of WO2012/139225A1).

In another embodiment, the PE-PilA fusion protein has at least 70%, 80%, 85%, 90%, 91%, 92%, 93%, 94%, 95%, 96%, 97%, 98%, 99%, or 100% identity to SEQ ID NO: 9 (LVL-735 wherein the signal peptide has been removed, corresponding to SEQ ID NO: 219 of WO2012/139225A1).

SEQ ID NO: 9: PE-PilA fusion protein without signal peptide

IQKAEQND VKLAPPTDVR SGYIRLVKNV NYYIDSESIW
VDNQEPQIVH FDAVVNLDKG LYVYPEPKRY ARSVRQYKIL
NCANYHLTQV RTDFYDEFWG QGLRAAPKKQ KKHTLSLTPD
TTLYNAAQII CANYGEAFSV DKKGGTKKAA VSELLQASAP
YKADVELCVY STNETTNCTG GKNGIAADIT TAKGYVKSVT
TSNGAITVKG DGTLANMEYI LQATGNAATG VTWTTTCKGT
DASLFPANFC GSVTQ

In an embodiment, the PE-PilA fusion protein comprises (or consists) of the amino acid sequence of SEQ ID NO: 9 (LVL-735 wherein the signal peptide has been removed, corresponding to SEQ ID NO: 219 of WO2012/139225A1).

The immunogenicity of immunogenic fragments of Protein E (PE) and Pilin A (PilA) may be measured as described in WO2012/139225A1.

### UspA2

Ubiquitous surface protein A2 (UspA2) is a trimeric autotransporter that appears as a lollipop-shared structure in electron micrographs (Hoiczyk et al. EMBO J. 19: 5989-5999 (2000)). It is composed of a N-terminal head, followed by a stalk which ends by an amphipathic helix and a C-terminal membrane domain. (Hoiczyk et al. EMBO J. 19: 5989-5999 (2000)). UspA2 contains a very well conserved domain (Aebi et al., Infection & Immunity 65(11) 4367-4377 (1997)), which is recognized by a monoclonal antibody that was shown protective upon passive transfer in a mouse Moraxella catarrhalis challenge model (Helminnen et al. J Infect Dis. 170(4): 867-72 (1994)). UspA2 has been shown to interact with host structures and extracellular matrix proteins like fibronectin (Tan et al., J Infect Dis. 192(6): 1029-38 (2005)) and laminin (Tan et al., J Infect Dis. 194(4): 493-7 (2006)), suggesting it can play a role at an early stage of Moraxella catarrhalis infection. UspA2 also seems to be involved in the ability of Moraxella catarrhalis to resist the bactericidal activity of normal human serum. (Attia A S et al. Infect Immun 73(4): 2400-2410 (2005)). It (i) binds the complement inhibitor C4 bp, enabling Moraxella catarrhalis to inhibit the classical complement system, (ii) prevents activation of the alternative complement pathway by absorbing C3 from serum and (iii) interferes with the terminal stages of the complement system, the Membrane Attack Complex (MAC), by binding the complement regulator protein vitronectin. (de Vries et al., Microbiol Mol Biol Rev. 73(3): 389-406 (2009)).

As used herein “UspA2” means Ubiquitous surface protein A2 from Moraxella catarrhalis. UspA2 may comprise (or consist) of the amino acid sequence of SEQ ID NO: 10 from ATCC 25238 (corresponding to SEQ ID NO: 1 of WO2015/125118A1):

(SEQ ID NO: 10)
MKTMKLLPLKIAVTSAMIIGLGAASTANAQAKNDITLEDLPYLIKKIDQN
ELEADIGDITALEKYLALSQYGNILALEELNKALEELDEDVGWNQNDIAN
LEDDVETLTKNQNALAEQGEAIKEDLQGLADFVEGQEGKILQNETSIKKN
TQRNLVNGFEIEKNKDAIAKNNESIEDLYDFGHEVAESIGEIHAHNEAQN
ETLKGLITNSIENTNNITKNKADIQALENNVVEELFNLSGRLIDQKADID
NNINNIYELAQQQDQHSSDIKTLKKNVEEGLLELSGHLIDQKTDIAQNQA
NIQDLATYNELQDQYAQKQTEAIDALNKASSENTQNIEDLAAYNELQDAY
AKQQTEAIDALNKASSENTQNIEDLAAYNELQDAYAKQQTEAIDALNKAS
SENTQNIAKNQADIANNINNIYELAQQQDQHSSDIKTLAKASAANTDRIA
KNKADADASFETLTKNQNTLIEKDKEHDKLITANKTAIDANKASADTKFA
ATADAITKNGNAITKNAKSITDLGTKVDGFDSRVTALDTKVNAFDGRITA
LDSKVENGMAAQAALSGLFQPYSVGKFNATAALGGYGSKSAVAIGAGYRV
NPNLAFKAGAAINTSGNKKGSYNIGVNYFF

as well as sequences having at least or exactly 63%, 66%, 70%, 72%, 74%, 75%, 77%, 80%, 84%, 85%, 90%, 95%, 96%, 97%, 98%, 99% or 100% identity, over the entire length, to SEQ ID NO: 10.

UspA2 polypeptides may be full length UspA2 or an immunogenic fragment thereof. In particular embodiments, the UspA2 polypeptide has at least 70%, 80%, 85%, 90%, 91%, 92%, 93%, 94%, 95%, 96%, 97%, 98%, 99%, or 100% identity to SEQ ID NO: 10. In another embodiment, the UspA2 polypeptide is an immunogenic fragment of UspA2 from Moraxella catarrhalis having at least 70%, 80%, 85%, 90%, 91%, 92%, 93%, 94%, 95%, 96%, 97%, 98%, or 99% identity to SEQ ID NO: 10. For example, immunogenic fragments of UspA2 may comprise at least 7, 10, 15, 20, 25, 30 or 50 contiguous amino acids of SEQ ID NO: 10. For example, immunogenic fragments of UspA2 may comprise at least 7, 10, 15, 20, 25, 30, 50, 100, 200, 300, 400, 500 or 60 contiguous amino acids of SEQ ID NO: 10, up to 629 contiguous amino acids of SEQ ID NO: 10. The immunogenic fragments may elicit antibodies which can bind SEQ ID NO: 10.

UspA2 as described in SEQ ID NO: 10 contains a signal peptide (for example, amino acids 1 to 29 of SEQ ID NO: 10), a laminin binding domain (for example, amino acids 30 to 177 of SEQ ID NO: 10), a fibronectin binding domain (for example, amino acids 165 to 318 of SEQ ID NO: 10) (Tan et al. JID 192: 1029-38 (2005)), a C3 binding domain (for example, amino acids 30 to 539 of SEQ ID NO: 10 (WO2007/018483), or a fragment of amino acids 30 to 539 of SEQ ID NO: 10, for example, amino acids 165 to 318 of SEQ ID NO: 1 (Hallström T et al. J. Immunol. 186: 3120-3129 (2011)), an amphipathic helix (for example, amino acids 519 to 564 of SEQ ID NO: 10 or amino acids 520-559 of SEQ ID NO:10, identified using different prediction methods) and a C terminal anchor domain (for example, amino acids 576 to 630 amino acids of SEQ ID NO: 10 (Brooks et al., Infection & Immunity, 76(11), 5330-5340 (2008)). In an embodiment, an UspA2 polypeptide contains a laminin binding domain and a fibronectin binding domain. In an additional embodiment, an immunogenic fragment of UspA2 contains a laminin binding domain, a fibronectin binding domain and a C3 binding domain. In a further embodiment, an UspA2 polypeptide, contains a laminin binding domain, a fibronectin binding domain, a C3 binding domain and an amphipathic helix.

UspA2 amino acid differences have been described for various Moraxella catarrhalis species. See for example, J Bacteriology 181(13):4026-34 (1999), Infection and Immunity 76(11):5330-40 (2008) and PLoS One 7(9):e45452 (2012). An UspA2 polypeptide, may comprise (or consist) of an amino acid sequence that differs from SEQ ID NO: 10 at any one or more amino acid selected from the group consisting of: AA (amino acid) 30 to 298, AA 299 to 302, AA 303 to 333, AA 334 to 339, AA 349, AA 352 to 354, AA 368 to 403, AA 441, AA 451 to 471, AA 472, AA474 to 483, AA 487, AA 490, AA 493, AA 529, AA 532 or AA 543. An UspA2 polypeptide, may comprise (or consist) of an amino acid sequence that differs from SEQ ID NO: 10 in that it contains an amino acid insertion in comparison to SEQ ID NO: 10. UspA2 may comprise (or consist) of an amino acid sequence that differs from SEQ ID NO: 10 at any one of the amino acid differences in SEQ ID NO: 22 through SEQ ID NO: 58. For example, SEQ ID NO: 10 may contain K instead of Q at amino acid 70, Q instead of G at amino acid 135 and/or D instead of N at amino acid 216.

UspA2 may be UspA2 from M. catarrhalis strain ATCC(a US registered trademark) 25238™, American 2933. American 2912, American 2908, Finnish 307, Finnish 353, Finnish 358, Finnish 216, Dutch H2, Dutch F10, Norwegian 1, Norwegian 13, Norwegian 20, Norwegian 25, Norwegian 27, Norwegian 36, BC5SV, Norwegian 14, Norwegian 3, Finish 414, Japanese Z7476, Belgium Z7530, German Z8063, American O12E, Greek MC317, American V1122, American P44, American V1171, American TTA24, American O35E, American SP12-6, American SP12-5, Swedish BC5, American 7169, Finnish FIN2344, American V1118, American V1145 or American V1156. UspA2 may be UspA2 as set forth in any of SEQ ID NO: 10 or SEQ ID NO: 22-SEQ ID NO: 38. UspA2 may be UspA2 from another source which corresponds to the sequence of UspA2 in any one of SEQ ID NO: 10 or SEQ ID NO: 22-SEQ ID NO: 58. Corresponding UspA2 sequences may be determined by one skilled in the art using various algorithms. For example, the Gap program or the Needle program may be used to determine UspA2 sequences corresponding to any one of SEQ ID NO: 10 or SEQ ID NO: 22-SEQ ID NO: 58.

UspA2 may be a sequence having at least 95% identity, over the entire length, to any of SEQ ID NO: 10 or SEQ ID NO: 22-SEQ ID NO: 58. In particular embodiments, UspA2 may be a sequence as set forth in an amino acid sequence selected from the group consisting of SEQ ID NO: 10, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 and SEQ ID NO: 58 or any subset of SEQ ID NO: 1 or SEQ ID NO:22 through SEQ ID NO:58.

Immunogenic fragments of UspA2 comprise immunogenic fragments of at least 450 contiguous amino acids of SEQ ID NO: 10, 490 contiguous amino acids of SEQ ID NO: 10 (for example, the UspA2 fragment of MC-004 or MC-005), 511 contiguous amino acids of SEQ ID NO: 10 (for example, the UspA2 fragment of construct MC-001, MC-002, MC-003 or MC-004), 534 contiguous amino acids of SEQ ID NO: 10 (for example, the UspA2 fragment of MC-009 or MC-011) or 535 contiguous amino acids of SEQ ID NO: 10 (for example, the UspA2 fragment of MC-007, MC-008 or MC-010). The immunogenic fragments may elicit antibodies which can bind SEQ ID NO: 10.

Immunogenic fragments of UspA2 may comprise immunogenic fragments of at least 450, 490, 511, 534 or 535 contiguous amino acids of SEQ ID NO: 10. For example, immunogenic fragments of UspA2 may comprise immunogenic fragments of at least 450, 490, 511, 534 or 535 contiguous amino acids of SEQ ID NO: 10, up to 629 amino acids of SEQ ID NO: 10. Immunogenic fragments of UspA2 may comprise immunogenic fragments of UspA2, for example any of the UspA2 constructs MC-001 (SEQ ID NO: 11), MC-002 (SEQ ID NO: 12), MC-003 (SEQ ID NO: 13), MC-004 (SEQ ID NO: 14), MC-005 (SEQ ID NO: 15), MC-006 (SEQ ID NO: 16), MC-007 (SEQ ID NO: 17), MC-008 (SEQ ID NO:18), MC-009 (SEQ ID NO: 19), MC-010 (SEQ ID NO: 20) or MC-011 (SEQ ID NO: 21). The immunogenic fragments may elicit antibodies which can bind the full length sequence from which the fragment is derived.

In another embodiment, the UspA2 polypeptide has at least 70%, 80%, 85%, 90%, 91%, 92%, 93%, 94%, 95%, 96%, 97%, 98%, 99%, or 100% identity to a polypeptide selected from the group consisting of MC-001 (SEQ ID NO: 11), MC-002 (SEQ ID NO: 12), MC-003 (SEQ ID NO: 13), MC-004 (SEQ ID NO: 14), MC-005 (SEQ ID NO: 15), MC-006 (SEQ ID NO: 16), MC-007 (SEQ ID NO: 17), MC-008 (SEQ ID NO:18), MC-009 (SEQ ID NO: 19), MC-010 (SEQ ID NO: 20) or MC-011 (SEQ ID NO: 21). For example, the UspA2 polypeptide has at least 70%, 80%, 85%, 90%, 91%, 92%, 93%, 94%, 95%, 96%, 97%, 98%, 99%, or 100% identity to MC009 SEQ ID NO: 19 (corresponding to SEQ ID NO: 69 of WO2015/125118A1).

SEQ ID NO: 19 MC-009 (Protein) - (M)(UspA2
31-564)(HH)
MAKNDITLEDLPYLIKKIDQNELEADIGDITALEKYLALSQYGNILALEE
LNKALEELDEDVGWNQNDIANLEDDVETLTKNQNALAEQGEAIKEDLQGL
ADFVEGQEGKILQNETSIKKNTQRNLVNGFEIEKNKDAIAKNNESIEDLY
DFGHEVAESIGEIHAHNEAQNETLKGLITNSIENTNNITKNKADIQALEN
NVVEELFNLSGRLIDQKADIDNNINNIYELAQQQDQHSSDIKTLKKNVEE
GLLELSGHLIDQKTDIAQNQANIQDLATYNELQDQYAQKQTEAIDALNKA
SSENTQNIEDLAAYNELQDAYAKQQTEAIDALNKASSENTQNIEDLAAYN
ELQDAYAKQQTEAIDALNKASSENTQNIAKNQADIANNINNIYELAQQQD
QHSSDIKTLAKASAANTDRIAKNKADADASFETLTKNQNTLIEKDKEHDK
LITANKTAIDANKASADTKFAATADAITKNGNAITKNAKSITDLGTKVDG
FDSRVTALDTKVNAFDGRITALDSKVENGMAAQAAHH

In an embodiment, the UspA2 polypeptide comprises (or consists) of an amino acid sequence of SEQ ID NO: 19 (corresponding to SEQ ID NO: 69 of WO2015/125118A1).

Immunogenicity of UspA2 polypeptides may be measured as described in WO2015/125118A1.

### Freeze Drying

The liquid composition of Protein D polypeptide prepared according to the process of the invention may subsequently be freeze-dried. Thus, the present invention provides a process comprising preparing a liquid composition comprising a Protein D polypeptide as described above and subsequently freeze-drying the liquid composition comprising the Protein D polypeptide. In an embodiment, the present invention provides a process for preparing a liquid composition comprising a Protein D polypeptide comprising steps (i), (ii), (iii), (iv) and subsequently comprising step of: (v) freeze-drying the liquid composition comprising the Protein D polypeptide. “Freeze-drying” refers to the process by which a suspension is frozen, after which the water is removed by sublimation. Sublimation is a change in the physical properties of a substance, wherein the solvent, e.g. water, in the substance changes directly from a solid (frozen) state to a gaseous state without becoming a liquid. Freeze drying is a low temperature dehydration process which involves freezing the formulation (e.g. an aqueous formulation) to below the triple point (the lowest temperature at which the solid, liquid and gas phases of the material can coexist), lowering pressure and removing ice (solid solvent) by sublimation in a primary drying step and removing remaining water in a second drying step. Annealing may optionally be used prior to drying to increase the size of the ice crystals by raising and lowering the temperature. Lyophilization is commonly used in vaccine manufacturing. In an embodiment, the immunogenic composition is lyophilized. Lyophilization is the process by which water is removed from a product after it is frozen and placed under a vacuum, allowing the ice to change directly from solid to vapor without passing through a liquid phase.

In an embodiment lyophilization is carried out using the following steps:

a freezing step (below the triple point)

optionally an annealing step

a primary drying step

a secondary drying step.

Lyophilization increases the concentration of components of a formulation in a process known as cryoconcentration.

Thus, the present invention provides a process for preparing a liquid composition comprising a Protein D polypeptide, which subsequently to the steps of mixing the Protein D polypeptide with sucrose and poloxamer (and optionally the step of filtration), the step of storing the liquid composition comprising the Protein D polypeptide and the step of mixing the liquid composition comprising the Protein D polypeptide with other antigen(s), comprises freeze-drying the liquid composition comprising the Protein D polypeptide. Thus, the process of the invention may comprise the steps (in sequential order): (i) thawing the Protein D polypeptide, (ii) mixing the Protein D polypeptide with sucrose and poloxamer (and optionally the step of filtration), (iii) storing the liquid composition comprising the Protein D polypeptide, (iv) mixing the liquid composition comprising the Protein D polypeptide with other antigen(s) and (v) freeze-drying the liquid composition comprising the Protein D polypeptide.

### Liquid Compositions of Protein D Polypeptide

The present invention provides a liquid composition comprising a Protein D polypeptide (optionally a Protein D polypeptide of SEQ ID NO: 2), sucrose and poloxamer (optionally poloxamer 188). In an embodiment, the present invention provides a liquid composition comprising a Protein D polypeptide (e.g. a Protein D polypeptide of SEQ ID NO: 2), optionally in an amount 0.025 to 20 mg/ml, 0.5 to 10 mg/ml, 0.5 to 1 mg/ml, or 1 mg/ml; sucrose, optionally in an amount 5 to 20% (w/v), 10 to 20% (w/v), or 10 to 15% (w/v); and poloxamer (e.g. poloxamer 188) optionally in an amount 0.1 to 1% (w/v), 0.5 to 1% (w/v), or 1% (w/v). In another embodiment, the present invention provides a liquid composition comprising a Protein D polypeptide (optionally a Protein D polypeptide of SEQ ID NO: 2), optionally in an amount 0.025 to 20 mg/ml, 0.5 to 10 mg/ml, 0.5 to img/ml, or 1 mg/ml; sucrose, optionally in an amount 5 to 20% (w/v), 10 to 20% (w/v), or 10 to 15% (w/v); poloxamer (optionally poloxamer 188) optionally in an amount 0.1 to 1% (w/v), 0.5 to 1% (w/v), or 1% (w/v); and a salt (optionally NaCl). In another embodiment, the present invention provides a liquid composition comprising a Protein D polypeptide (optionally a Protein D polypeptide of SEQ ID NO: 2), optionally in an amount 0.025 to 20 mg/ml, 0.5 to 10 mg/ml, 0.5 to 1 mg/ml, or 1 mg/ml; sucrose, optionally in an amount 5 to 20% (w/v), 10 to 20% (w/v), or 10 to 15% (w/v); poloxamer (optionally poloxamer 188) optionally in an amount 0.1 to 1% (w/v), 0.5 to 1% (w/v), or 1% (w/v); a buffer (optionally phosphate buffer); and a salt (optionally NaCl).

The above described ranges for the amounts of Protein D polypeptide, sucrose and poloxamer may be combined. For example, the present invention provides a liquid compositions comprising a Protein D polypeptide (e.g. a Protein D polypeptide of SEQ ID NO: 2), sucrose and poloxamer (e.g. poloxamer 188) comprising: Protein D polypeptide in an amount 0.025 to 20 mg/ml; sucrose, in an amount 5 to 20% (w/v); poloxamer (e.g. poloxamer 188) in an amount 0.1 to 1% (w/v). For example, the present invention provides a liquid compositions comprising a Protein D polypeptide (e.g. a Protein D polypeptide of SEQ ID NO: 2), sucrose and poloxamer (e.g. poloxamer 188) comprising: Protein D polypeptide in an amount 0.5 to 10 mg/ml; sucrose, in an amount 5 to 20% (w/v); poloxamer (e.g. poloxamer 188) in an amount 0.1 to 1% (w/v). For example, the present invention provides a liquid compositions comprising a Protein D polypeptide (e.g. a Protein D polypeptide of SEQ ID NO: 2), sucrose and poloxamer (e.g. poloxamer 188) comprising: Protein D polypeptide in an amount 0.025 to 20 mg/ml; sucrose, in an amount 10 to 20% (w/v); poloxamer (e.g. poloxamer 188) in an amount 0.1 to 1% (w/v). For example, the present invention provides a liquid compositions comprising a Protein D polypeptide (e.g. a Protein D polypeptide of SEQ ID NO: 2), sucrose and poloxamer (e.g. poloxamer 188) comprising: Protein D polypeptide in an amount 0.5 to 10 mg/ml; sucrose, in an amount 10 to 20% (w/v); poloxamer (e.g. poloxamer 188) in an amount 0.5 to 1% (w/v). For example, the present invention provides a liquid compositions comprising a Protein D polypeptide (e.g. a Protein D polypeptide of SEQ ID NO: 2), sucrose and poloxamer (e.g. poloxamer 188) comprising: Protein D polypeptide in an amount 0.5 to img/ml; sucrose, in an 10 to 15% (w/v); poloxamer (e.g. poloxamer 188) in an amount 0.5 to 1% (w/v). For example, the present invention provides a liquid compositions comprising a Protein D polypeptide (e.g. a Protein D polypeptide of SEQ ID NO: 2), sucrose and poloxamer (e.g. poloxamer 188) comprising: Protein D polypeptide in an amount 0.025 to 20 mg/ml; sucrose, in an amount 5 to 20% (w/v); poloxamer (e.g. poloxamer 188) in an amount 0.1 to 1% (w/v), a buffer (e.g. phosphate buffer). For example, the present invention provides a liquid compositions comprising a Protein D polypeptide (e.g. a Protein D polypeptide of SEQ ID NO: 2), sucrose and poloxamer (e.g. poloxamer 188) comprising: Protein D polypeptide in an amount 0.5 to 10 mg/ml; sucrose, in an amount 5 to 20% (w/v); poloxamer (e.g. poloxamer 188) in an amount 0.1 to 1% (w/v), a buffer (e.g. phosphate buffer). For example, the present invention provides a liquid compositions comprising a Protein D polypeptide (e.g. a Protein D polypeptide of SEQ ID NO: 2), sucrose and poloxamer (e.g. poloxamer 188) comprising: Protein D polypeptide in an amount 0.025 to 20 mg/ml; sucrose, in an amount 10 to 20% (w/v); poloxamer (e.g. poloxamer 188) in an amount 0.1 to 1% (w/v), a buffer (e.g. phosphate buffer). For example, the present invention provides a liquid compositions comprising a Protein D polypeptide (e.g. a Protein D polypeptide of SEQ ID NO: 2), sucrose and poloxamer (e.g. poloxamer 188) comprising: Protein D polypeptide in an amount 0.5 to 10 mg/ml; sucrose, in an amount 10 to 20% (w/v); poloxamer (e.g. poloxamer 188) in an amount 0.5 to 1% (w/v), a buffer (e.g. phosphate buffer). For example, the present invention provides a liquid compositions comprising a Protein D polypeptide (e.g. a Protein D polypeptide of SEQ ID NO: 2), sucrose and poloxamer (e.g. poloxamer 188) comprising: Protein D polypeptide in an amount 0.5 to 1 mg/ml; sucrose, in an 10 to 15% (w/v); poloxamer (e.g. poloxamer 188) in an amount 0.5 to 1% (w/v), a buffer (e.g. phosphate buffer). For example, the present invention provides a liquid compositions comprising a Protein D polypeptide (e.g. a Protein D polypeptide of SEQ ID NO: 2), sucrose and poloxamer (e.g. poloxamer 188) comprising: Protein D polypeptide in an amount 0.025 to 20 mg/ml; sucrose, in an amount 5 to 20% (w/v); poloxamer (e.g. poloxamer 188) in an amount 0.1 to 1% (w/v), a buffer (e.g. phosphate buffer) and a salt (e.g. NaC). For example, the present invention provides a liquid compositions comprising a Protein D polypeptide (e.g. a Protein D polypeptide of SEQ ID NO: 2), sucrose and poloxamer (e.g. poloxamer 188) comprising: Protein D polypeptide in an amount 0.5 to 10 mg/ml; sucrose, in an amount 5 to 20% (w/v); poloxamer (e.g. poloxamer 188) in an amount 0.1 to 1% (w/v), a buffer (e.g. phosphate buffer) and a salt (e.g. NaC). For example, the present invention provides a liquid compositions comprising a Protein D polypeptide (e.g. a Protein D polypeptide of SEQ ID NO: 2), sucrose and poloxamer (e.g. poloxamer 188) comprising: Protein D polypeptide in an amount 0.025 to 20 mg/ml; sucrose, in an amount 10 to 20% (w/v); poloxamer (e.g. poloxamer 188) in an amount 0.1 to 1% (w/v), a buffer (e.g. phosphate buffer) and a salt (e.g. NaCl). For example, the present invention provides a liquid compositions comprising a Protein D polypeptide (e.g. a Protein D polypeptide of SEQ ID NO: 2), sucrose and poloxamer (e.g. poloxamer 188) comprising: Protein D polypeptide in an amount 0.5 to 10 mg/ml; sucrose, in an amount 10 to 20% (w/v); poloxamer (e.g. poloxamer 188) in an amount 0.5 to 1% (w/v), a buffer (e.g. phosphate buffer) and a salt (e.g. NaCl). For example, the present invention provides a liquid compositions comprising a Protein D polypeptide (e.g. a Protein D polypeptide of SEQ ID NO: 2), sucrose and poloxamer (e.g. poloxamer 188) comprising: Protein D polypeptide in an amount 0.5 to 1 mg/ml; sucrose, in an 10 to 15% (w/v); poloxamer (e.g. poloxamer 188) in an amount 0.5 to 1% (w/v), a buffer (e.g. phosphate buffer) and a salt (e.g. NaCl).

In an embodiment, the present invention provides a liquid composition comprising a Protein D polypeptide (e.g. a Protein D polypeptide of SEQ ID NO: 2), poloxamer (e.g. poloxamer 188) and sucrose prepared by a process of the invention. In an embodiment, the present invention provides a liquid composition comprising a Protein D polypeptide which is stable. Suitably, the liquid composition comprising a Protein D polypeptide is stable for at least 1 day, at least 7 days or at least 14 days. In some embodiments, the liquid composition comprising a Protein D polypeptide is stable for at least 1, 2, 3, 4, 5, 6, 7 or 14 days. For example, the liquid composition comprising a Protein D polypeptide may be stable for at least 1 day, suitably up to 7 days (e.g. between 1 to 7 days), or up to 14 days (e.g. between 1 to 14 days). In an embodiment, the present invention provides a liquid composition comprising a Protein D polypeptide, poloxamer and sucrose, which has fewer visible particles, compared to a liquid composition comprising a Protein D polypeptide without poloxamer and without sucrose, when maintained as a liquid composition for at least 1, 2, 3, 4, 5, 6, 7 or 14 days.

In an embodiment, the liquid composition comprising Protein D polypeptide of the present invention does not contain visible particles. In an embodiment, the liquid composition comprising Protein D polypeptide of the present invention does not contain visible particles when maintained as a liquid composition for at least 1 day. In an embodiment, the liquid composition comprising Protein D polypeptide of the present invention does not contain visible particles when maintained as a liquid composition for at least 7 days. In an embodiment, the liquid composition comprising Protein D polypeptide of the present invention does not contain visible particles when maintained as a liquid composition for at least 14 days. For example, the liquid composition comprising a Protein D polypeptide does not contain visible particles when maintained as a liquid composition for at least 1 day, suitably up to 7 days (e.g. between 1 to 7 days), or up to 14 days (e.g. between 1 to 14 days). In an embodiment, the liquid composition comprising Protein D polypeptide of the present invention contains less than 100 particles within the size range 50 to 1000 μm according to flow camera (Occhio) particle counting (as described herein). In an embodiment, the liquid composition comprising a Protein D polypeptide contains less than 100 particles within the size range 50 to 1000 μm according to Occhio particle counting when maintained as a liquid composition for at least 1 day. In an embodiment the liquid composition comprising a Protein D polypeptide contains less than 100 particles within the size range 50 to 1000 μm according to Occhio particle counting when maintained as a liquid composition for at least 7 days. In an embodiment, the liquid composition comprising Protein D polypeptide contains less than 100 particles within the size range 50 to 1000 μm according to Occhio particle counting when maintained as a liquid composition for at least 14 days. In an embodiment, the liquid composition comprising Protein D polypeptide contains less than 100 particles within the size range 50 to 1000 μm according to Occhio particle counting when maintained as a liquid composition for at least 1 day, suitably up to 7 days (e.g. between 1 to 7 days), or up to 14 days (e.g. between 1 to 14 days).

### Uses, and Methods of Treatment and Prevention

The present invention also provides an immunogenic composition wherein the Protein D polypeptide has been prepared using a process of the invention. The immunogenic composition may further comprise Protein E from Haemophilus influenzae or an immunogenic fragment thereof, PilA from Haemophilus influenzae or an immunogenic fragment thereof and a UspA2 polypeptide from Moraxella catarrhalis. In another embodiment, the immunogenic composition may further comprise a PE-PilA fusion protein and an UspA2 polypeptide. The immunogenic composition may be used in the treatment or prevention of a disease caused by H. influenzae and/or M. catarrhalis or for the treatment or prevention of an acute exacerbation of COPD (AECOPD) in a subject, e.g. human.

An immunogenic composition of the invention may further comprise a pharmaceutically acceptable adjuvant. Suitable adjuvants include an aluminum salt such as aluminum hydroxide gel or aluminum phosphate or alum, but may also be a salt of calcium, magnesium, iron or zinc, or may be an insoluble suspension of acylated tyrosine, or acylated sugars, cationically or anionically derivatized saccharides, or polyphosphazenes. In particular embodiments, the protein antigen may be adsorbed onto aluminium phosphate. In another embodiment, the protein antigen may be adsorbed onto aluminium hydroxide. Suitable adjuvant systems which promote a predominantly Th1 response also include: non-toxic derivatives of lipid A, Monophosphoryl lipid A (MPL) or a derivative thereof, particularly 3-de-O-acylated monophosphoryl lipid A (3D-MPL) (for its preparation see GB 2220211 A); and a combination of monophosphoryl lipid A, e.g. 3-de-O-acylated monophosphoryl lipid A, together with either an aluminum salt (for instance aluminum phosphate or aluminum hydroxide) or an oil-in-water emulsion. In such combinations, antigen and 3D-MPL are contained in the same particulate structures, allowing for more efficient delivery of antigenic and immunostimulatory signals. Studies have shown that 3D-MPL is able to further enhance the immunogenicity of an alum-adsorbed antigen (Thoelen et al. Vaccine (1998) 16:708-14; EP 689454-B1). For example, the pharmaceutically acceptable adjuvant may be AS01. AS01 is an Adjuvant System containing MPL (3-O-desacyl-4′-monophosphoryl lipid A), QS21 ((Quillaja saponaria Molina, fraction 21) Antigenics, New York, N.Y., USA) and liposomes. AS01B is an Adjuvant System containing MPL, QS21 and liposomes (50 μg MPL and 50 μg QS21). AS01E is an Adjuvant System containing MPL, QS21 and liposomes (25 μg MPL and 25 μg QS21). The immunogenic composition or vaccine of the invention may comprise AS01, e.g. AS01B or AS01E.

The present invention thus provides an immunogenic composition for use in the treatment or prevention of a disease caused by H. influenzae and/or M. catarrhalis. The present invention also provides use of an immunogenic composition of the invention, in the manufacture of a medicament for the treatment or prevention of a disease caused by H. influenzae and/or M. catarrhalis. In addition, the present invention provides a method of treatment or prevention of a disease caused by H. influenzae and/or M. catarrhalis in a subject, e.g. human, at risk, said method comprising administering to said subject, an effective amount of an immunogenic composition of the invention. In addition, the present invention provides a method of prevention of a disease caused by H. influenzae and/or M. catarrhalis in a subject, e.g. human, at risk, said method comprising administering to said subject, an effective amount of an immunogenic composition of the invention. In addition, the present invention provides a method of treatment of a disease caused by H. influenzae and/or M. catarrhalis in a subject, e.g. human, at risk, said method comprising administering to said subject, an effective amount of an immunogenic composition of the invention. In addition, the present invention provides a method of inducing an immune response to H. influenzae and/or M. catarrhalis in a subject (e.g. human), said method comprising administering to said subject, an effective amount of an immunogenic composition of the invention.

The present invention provides an immunogenic composition of the invention for use in the treatment or prevention of an acute exacerbation of COPD (AECOPD) in a subject, e.g. human. The present invention also provides use of an immunogenic composition of the invention, in the manufacture of a medicament for the treatment or prevention of an acute exacerbation of COPD (AECOPD). In addition, the present invention provides a method of treatment or prevention of an acute exacerbation of COPD (AECOPD) in a subject, e.g. human, at risk of developing an acute exacerbation of COPD (AECOPD), said method comprising administering to said subject, an effective amount of an immunogenic composition of the invention. In addition, the present invention provides a method of prevention of an acute exacerbation of COPD (AECOPD) in a subject, e.g. human, at risk of developing an acute exacerbation of COPD (AECOPD), said method comprising administering to said subject, an effective amount of an immunogenic composition of the invention. In addition, the present invention provides a method of treatment of an acute exacerbation of COPD (AECOPD) in a subject, e.g. human, at risk of developing an acute exacerbation of COPD (AECOPD), said method comprising administering to said subject, an effective amount of an immunogenic composition of the invention.

Chronic obstructive pulmonary disease (COPD) is a lung disease characterized by chronic obstruction of lung airflow that interferes with normal breathing and is not fully reversible. A COPD diagnosis is confirmed by a simple test called spirometry, which measures how deeply a person can breathe and how fast air can move into and out of the lungs. Such a diagnosis should be considered in any patient who has symptoms of cough, sputum production, or dyspnea (difficult or labored breathing), and/or a history of exposure to risk factors for the disease. Where spirometry is unavailable, the diagnosis of COPD should be made using all available tools. Clinical symptoms and signs, such as abnormal shortness of breath and increased forced expiratory time, can be used to help with the diagnosis. A low peak flow is consistent with COPD, but may not be specific to COPD because it can be caused by other lung diseases and by poor performance during testing. Chronic cough and sputum production often precede the development of airflow limitation by many years, although not all individuals with cough and sputum production go on to develop COPD.

An acute exacerbation of COPD (AECOPD) is an acute event characterised by a worsening of the patient's respiratory symptoms that is beyond normal day-to-day variations. Typically an AECOPD leads to a change in medication. Acute exacerbations and comorbidities contribute to the overall disease severity in individual COPD patients. An acute exacerbation of COPD (AECOPD) is an acute event characterised by a worsening of the patient's respiratory symptoms that is beyond normal day-to-day variations and leads to a change in medication [Perez A C, Murphy T F. Potential impact of a Moraxella catarrhalis vaccine in COPD. Vaccine. 2017]. AECOPD increases morbidity and mortality, leading to faster decline in lung function, poorer functional status [Sapey E, Stocley R A. COPD exacerbations. 2: aetiology. Thorax. 2006; 61(3):250-8)]. The lungs are known to be colonised with different species of bacteria [Erb-Downward J R, et al. PLoS One. 2011; 6(2):e16384 and Wilkinson T M A, et al. Thorax. 2017; 72(10):919-27]. In COPD patients, acquisition of new bacterial strains is believed to be an important cause of AECOPD [Seti S, et al. N Engl J Med. 2002; 347(7):465-71]. Although estimates vary widely, Non-Typeable Haemophilus influenzae (NTHi) appears to be the main bacterial pathogen associated with AECOPD (11-38%), followed by Moraxella catarrhalis (3-25%) and Streptococcus pneumoniae (4-9%) [Alamoudi O S. et al. Respirology. 2007; 12(2):283-7, Bandi V, et al. FEMS Immunol Med Microbiol. 2003; 37(1):69-75, Beasley V, et al. Int J Chron Obstuct Pulmon Dis. 2012; 7:555-69]. In an embodiment, the acute exacerbation of chronic obstructive pulmonary disease (AECOPD) is associated with a bacterial infection in a subject, e.g. a bacterial infection of Haemophilus influenzae (e.g. non-typeable H. influenzae (NTHI)) and/or Moraxella catarrhalis. In another embodiment, the bacterial infection is present in the lung(s) of a subject, e.g. human. In another embodiment, the subject, e.g. human, is at risk for developing an acute exacerbation of chronic obstructive pulmonary disease (AECOPD) resulting from a bacterial infection.

### Presentation

In certain embodiments, the immunogenic composition is contained within a container means e.g. a vial, or a syringe, including a pre-filled syringe. In certain embodiments, the container means is siliconized. Where an immunogenic composition of the invention is presented in a vial, this is suitably made of a glass or plastic material. The vial is preferably sterilized before the composition is added to it. The vial may include a single dose of vaccine, or it may include more than one dose (a ‘multidose’ vial) e.g. 10 doses. When using a multidose vial, each dose should be withdrawn with a sterile needle and syringe under strict aseptic conditions, taking care to avoid contaminating the vial contents. A vial can have a cap (e.g. a Luer lock) adapted such that a pre-filled syringe can be inserted into the cap, the contents of the syringe can be expelled into the vial (e.g. to reconstitute lyophilised material therein), and the contents of the vial can be removed back into the syringe. After removal of the syringe from the vial, a needle can then be attached and the composition can be administered to a patient. The cap is preferably located inside a seal or cover, such that the seal or cover has to be removed before the cap can be accessed.

Immunogenic compositions of the invention may be adapted for administration by an appropriate route, for example, by the intramuscular route.

In another embodiment, the present invention provides a vaccine comprising the immunogenic composition of the invention.

Embodiments of the invention are further described in the subsequent numbered paragraphs:
1. A process for preparing a liquid composition comprising a Protein D polypeptide (optionally a Protein D polypeptide of SEQ ID NO: 2), wherein the process comprises mixing the Protein D polypeptide with sucrose and poloxamer.
2. A process for preparing a liquid composition comprising a Protein D polypeptide according to paragraph 1 wherein the process comprises mixing the Protein D polypeptide with sucrose and poloxamer prior to mixing the Protein D polypeptide with other antigens.
3. A process for preparing a liquid composition comprising a Protein D polypeptide according to paragraph 1 or paragraph 2, wherein the process comprises mixing the Protein D polypeptide with solution(s) comprising: (a) sucrose to a concentration of 5 to 20% (w/v), 10 to 20% (w/v), or 10 to 15% (w/v), and (b) poloxamer (optionally poloxamer 188) to a concentration of 0.1 to 1% (w/v), 0.5 to 1% (w/v), or 1% (w/v).
4. A process for preparing a liquid composition comprising a Protein D polypeptide according to any of paragraphs 1 to 3, wherein the process comprises mixing the Protein D polypeptide with solution(s) comprising: (a) sucrose, (b) poloxamer (optionally poloxamer 188) and (c) a salt (optionally NaC).
5. A process for preparing a liquid composition comprising a Protein D polypeptide according to any of paragraphs 1 to 3, wherein the process comprises mixing the Protein D polypeptide with solution(s) comprising: (a) sucrose, (b) poloxamer (optionally poloxamer 188), (c) a salt (optionally NaCl) and (d) a buffer (optionally phosphate buffer).
6. A process for preparing a liquid composition comprising a Protein D polypeptide according to any of paragraphs 1 to 3, wherein the process comprises mixing the Protein D polypeptide with solution(s) comprising: (a) sucrose, (b) poloxamer (optionally poloxamer 188) (c) a salt, optionally NaCl, and (d) a buffer (optionally phosphate buffer), to reach a pH6.4 to 7.7 (e.g. pH6.8).
7. A process for preparing a liquid composition comprising a Protein D polypeptide according to any of paragraphs 1 to 6, wherein the process comprises the steps of: (i) thawing the Protein D polypeptide, and (ii) mixing the Protein D polypeptide with sucrose and poloxamer.
8. A process for preparing a liquid composition comprising a Protein D polypeptide according to any of paragraphs 1 to 7, subsequently comprising step of filtration (optionally using a 0.22 μm PVDF membrane) to obtain a liquid composition comprising the Protein D polypeptide in the filtrate.
9. A process for preparing a liquid composition comprising a Protein D polypeptide according to any of paragraphs 1 to 8, subsequently comprising the step of storing the liquid composition comprising the Protein D polypeptide.
10. A process for preparing a liquid composition comprising a Protein D polypeptide according to any of paragraphs 1 to 9, subsequently comprising the step of mixing the liquid composition comprising the Protein D polypeptide with other antigen(s).
11. A process for preparing a liquid composition comprising a Protein D polypeptide according to paragraph 10, wherein the other antigens comprise a PE-PilA fusion protein and an UspA2 polypeptide.
12. A process for preparing a liquid composition comprising a Protein D polypeptide according to any of paragraphs 1 to 11, which reduces the formation of Protein D polypeptide visible particles.
13. A process comprising preparing a liquid composition comprising a Protein D polypeptide according to the process of any of paragraphs 1 to 12 and subsequently freeze-drying the liquid composition comprising the Protein D polypeptide.
14. A liquid composition comprising a Protein D polypeptide (optionally a Protein D polypeptide of SEQ ID NO: 2), sucrose and poloxamer (optionally poloxamer 188).
15. A liquid composition according to paragraph 14 comprising a Protein D polypeptide (optionally a Protein D polypeptide of SEQ ID NO: 2), optionally in an amount 0.025 to 20 mg/ml, 0.5 to 10 mg/ml, 0.5 to 1 mg/ml, or 1 mg/ml; sucrose, optionally in an amount 5 to 20% (w/v), 10 to 20% (w/v), or 10 to 15% (w/v); poloxamer (optionally poloxamer 188) optionally in an amount 0.1 to 1% (w/v), 0.5 to 1% (w/v), or 1% (w/v); a buffer (optionally phosphate buffer); and a salt (optionally NaCl).

In order that this invention may be better understood, the following examples are set forth. These examples are for purposes of illustration only and are not to be construed as limiting the scope of the invention in any manner.

### EXAMPLES

### Analytical Techniques

### Light Obscuration

Light Obscuration is the compendial method of choice listed in the pharmacopeias (Ph. Eur. 2.9.19 and USP (United States Pharmacopeia)<788>) for the analysis of subvisible particles in parenteral products. The detection range of particle sizes is between 2 and 175 μm. The required volume is about 5 ml. In order to ensure that particulates detected by Light Obscuration do not come from the media, the media are analysed at day 1 by Light Obscuration at their maximum concentration, i.e. sucrose 10%, Poloxamer 188 1%, NaCl 150 mM and PO₄ buffer 12.5 mM. The equipment used was an APS-2000 (Automated Parenteral Sampling System)

The hardware components for the APSS-2000, consists of two central components:
Particle Counter model LiQuilaz® E20P
The Syringe Sampler model SLS-1000

The LiQuilaz-E20P particle counter uses light extinction for the measurement and classification particles. When a particle crosses the light source (laser diode) it creates a momentary obscuration of light. This obscuration of light is transformed into an electronic signal, which can be directly correlated to the size of a transient particle. Using preset algorithms the distribution of particle can be defined. The syringe sampler is used to pull a sample through the optics chamber at a pre-defined and fixed flow rate.

Parameters used for the analysis were:
1 milliliter of sample is analysed 4 times (total of 4 ml) (the first measurement is discarded)
Flow rate 10 ml/min

### Occhio

Occhio is an emerging technique developed to monitor, measure, and visualize sub-visible and visible particulates. It integrates digital microscopy, micro-fluidics and image processing into a single instrument for automatic analysis of particles or cells suspended in liquids. It operates by capturing images from the sample as it passes through the flow cell's sensing zone. Every particle in each image is analysed to create a database of particle counts, size, transparency and morphology (or shape). For immediate visual verification, images are displayed on the system monitor in real-time. The detection range of particle sizes is between 0.4 and 1000 μm. A volume of around 2 ml is tested. Occhio (IPAC2) was chosen to analyse fibers aggregates with the optimised following main parameters hardware configuration:
400 μm cell
1 ml syringe
4× Zoom

### Protein D Content by RP UPLC

The specific Antigen content was evaluated by a reverse phase high performance liquid chromatography (RP-HPLC) method using a Zorbax 300 SBC3 4.6×50 mm 3.5 μm column with a guard column 4.6×12.5 mm coupled with a UV detector set at 215 nm. The Protein D was eluted at approximately 9 minutes.

### Circular Dichroism (CD) Spectroscopy

FAR-UV CD: The ellipticity (mdeg), calculated based on the difference in the absorption of left-handed circularly polarized light (L-CPL) and right-handed circularly polarized light (R-CPL) was measured between 200 and 265 nm, which corresponds to the absorbance region of the peptide links. The signal obtained was thus linked to the secondary structural composition of the antigens such as the α-helix and the β sheet. FAR-UV CD was used to detect a modification of the secondary structure (α helix, β sheet . . . ).
Near-UV CD: was needed to detect the modification of the tertiary structure of the protein linked to a change of environment of the aromatic amino acids.

### ATR-FTIR

ATR-FTIR method is based on reflectance. IR radiation is directed to a crystal with a high refractive index that is in contact with the sample. The beam is reflected inside the crystal before being directed to the detector. When the beam hits the reflecting surface, it is partially absorbed and the incident bean is recorded.

The infrared spectrum of proteins contains contributions from the peptide amide group, called amide I, II, etc. . . . and from relatively weaker contributions from the amino acid side chains. The Amide II band (1550-1450 cm⁻¹) is assigned predominantly to the δ N—H of the peptide bond. In the 1700-1600 cm⁻¹, the Amide I band, assigned to uC=O of the peptide bond, is by far the most sensitive to the protein secondary structure. Because the strength of the hydrogen bonds existing within each secondary structure is different, each secondary structure absorbs at different wavelengths within the amide I region. The frequency limits for each secondary structure have been assigned based on theoretical and experimental data (Goormaghtigh et al, 2006, Evaluation of the Information Content in Infrared Spectra for Protein Secondary Structure Determination; Biophysical Journal, 90(8) 2946-2957): 1662-1645 cm⁻¹ for α-helix, 1689-1682 cm⁻¹ for β-sheets, 1644-1637 cm⁻¹ for random and 1682-1662 for β-turn.

### Intrinsic Fluorescence

The Fluorescence emission (A.U.) of a protein is related to its aromatic amino acids content and mainly to the contribution of tryptophan and tyrosine residues. The signal obtained is linked to the more or less polar environment of these chromophores and thus to their position in the protein. The Fluorescence spectrum shape, with its maximum, is then related to the tertiary structure of the protein.

### Example 1: Screening of Excipients and their Impact on Particle Formation During Liquid Storage (Part 1)

The study goal was to identifying excipients and/or parameters which have a positive impact on the colloidal stability of Protein D in liquid state at 2-8° C. A full factorial screening study was performed in order to determine which parameters had a positive or negative impact on the apparition of visible particles. The studied parameters were:
Protein D concentration (2 levels)
0.5 mg/ml
1 mg/ml
pH (2 levels)
6.8
7.7
Presence of Sucrose (2 levels)
0% m/v sucrose
10% m/v sucrose
Presence of Poloxamer 188 (2 levels)
0%
0.5%
Presence of NaCl (2 levels)
0 mM
150 mM

The frozen Protein D was thawed at 25° C. in an incubator (1 h 30). After thawing, the Protein D was diluted to 20 mg/mi in 150 mM NaCl and then filtered on a 0.22 μm Millipore Millex™ Sterile Syringe Filter (SLGV033RS). Afterwards, the 37 conditions were formulated by Tecan®. These conditions correspond to a full factorial study (32 samples, see Table 1) with in addition 3 times the central point (0.75 mg/ml Protein D, 75 mM NaCl, 5% sucrose, 0.25% Poloxamer 188 and pH 7.4) and 2 times the actual process (1 mg/mi PD in 150 mM NaCl)). Formulations were performed in PEN glass containers (non siliconized) (2×10 ml per formulation). The two PEN containers were pooled in a single Duran Schott container (non siliconized) (20 ml), stored at 2-8° C. for (the different time points (1 day, 7 days, 14 days and 21 days). For time point 7 days and 14 days, a control without visible particles was added to the Light Obscuration measurements. This control was the reference (actual process: 1 mg/ml Protein D in 150 mM NaCl)) filtered during the day. After 21 days, the control without visible particles was not analysed, because enough data were generated at time point 7 and 14 days.

### Visual Inspection

All visual inspections were performed by the same person at each time point (days 1, 7, 14 and 21) (see Table 1). The visual inspections were performed in the lab and not in a black and white visual inspection post. The aim was to define conditions that allow reducing or deleting the apparition of visible particles (±50 μm).

TABLE 1
Visual inspections at the four time points (T 1 day, T 7 days, T 14 days and T 21 days).
− means no particles, + means few particles and ++ means lot of particles (This classification
is the appreciation of the person who performed the visual inspection)

| | | | | Protein | | Visual | Visual | Visual | Visual |
|---|---|---|---|---|---|---|---|---|---|
| | Sucrose | NaCl | Poloxamer | D | Target | obs. | obs. | obs. day | obs. day |
| # Samples | (%) | (mM) | 188 (%) | (mg/ml) | pH | day 1 | day 7 | 14 | 21 |
| 17C0P03027 | 0 | 0 | 0 | 0.5 | 6.8 | − | + | + | + |
| 17C0P03006 | 0 | 0 | 0 | 0.5 | 7.7 | − | + | ++ | ++ |
| 17C0P03004 | 0 | 0 | 0 | 1 | 6.8 | + | + | + | ++ |
| 17C0P03018 | 0 | 0 | 0 | 1 | 7.7 | + | + | ++ | ++ |
| 17C0P03001 | 0 | 0 | 0.5 | 0.5 | 6.8 | − | + | + | + |
| 17C0P03028 | 0 | 0 | 0.5 | 0.5 | 7.7 | − | + | + | + |
| 17C0P03025 | 0 | 0 | 0.5 | 1 | 6.8 | − | + | + | + |
| 17C0P03012 | 0 | 0 | 0.5 | 1 | 7.7 | − | + | + | + |
| 17C0P03019 | 0 | 150 | 0 | 0.5 | 6.8 | + | + | ++ | ++ |
| 17C0P03015 | 0 | 150 | 0 | 0.5 | 7.7 | + | + | ++ | ++ |
| 17C0P03007 | 0 | 150 | 0 | 1 | 6.8 | + | ++ | ++ | ++ |
| 17C0P03033 | 0 | 150 | 0 | 1 | 6.8 | − | + | ++ | ++ |
| 17C0P03037 | 0 | 150 | 0 | 1 | 6.8 | + | + | ++ | ++ |
| 17C0P03022 | 0 | 150 | 0 | 1 | 7.7 | + | ++ | ++ | ++ |
| 17C0P03003 | 0 | 150 | 0.5 | 0.5 | 6.8 | − | − | + | + |
| 17C0P03009 | 0 | 150 | 0.5 | 0.5 | 7.7 | − | − | + | + |
| 17C0P03024 | 0 | 150 | 0.5 | 1 | 6.8 | − | − | + | + |
| 17C0P03013 | 0 | 150 | 0.5 | 1 | 7.7 | − | − | ++ | ++ |
| 17C0P03011 | 5 | 75 | 0.25 | 0.75 | 7.4 | − | + | + | + |
| 17C0P03016 | 5 | 75 | 0.25 | 0.75 | 7.4 | − | + | + | + |
| 17C0P03031 | 5 | 75 | 0.25 | 0.75 | 7.4 | − | − | + | + |
| 17C0P03017 | 10 | 0 | 0 | 0.5 | 6.8 | − | + | + | + |
| 17C0P03008 | 10 | 0 | 0 | 0.5 | 7.7 | + | + | ++ | ++ |
| 17C0P03035 | 10 | 0 | 0 | 1 | 6.8 | + | + | + | + |
| 17C0P03036 | 10 | 0 | 0 | 1 | 7.7 | − | + | ++ | ++ |
| 17C0P03021 | 10 | 0 | 0.5 | 0.5 | 6.8 | + | + | + | + |
| 17C0P03020 | 10 | 0 | 0.5 | 0.5 | 7.7 | − | + | + | + |
| 17C0P03014 | 10 | 0 | 0.5 | 1 | 6.8 | + | + | + | + |
| 17C0P03034 | 10 | 0 | 0.5 | 1 | 7.7 | − | − | + | + |
| 17C0P03023 | 10 | 150 | 0 | 0.5 | 6.8 | + | + | ++ | ++ |
| 17C0P03002 | 10 | 150 | 0 | 0.5 | 7.7 | − | + | + | + |
| 17C0P03026 | 10 | 150 | 0 | 1 | 6.8 | + | + | + | + |
| 17C0P03029 | 10 | 150 | 0 | 1 | 7.7 | − | + | ++ | ++ |
| 17C0P03030 | 10 | 150 | 0.5 | 0.5 | 6.8 | + | + | + | + |
| 17C0P03010 | 10 | 150 | 0.5 | 0.5 | 7.7 | − | + | + | + |
| 17C0P03032 | 10 | 150 | 0.5 | 1 | 6.8 | − | − | + | + |
| 17C0P03005 | 10 | 150 | 0.5 | 1 | 7.7 | − | + | ++ | ++ |

As seen in the Table 1 above, visible particles were present in some samples already after 24 h of storage at 2/8° C. After 7 days of storage at 2/8° C., only 20% of samples were free of visible particles. For the time points 14 and 21 days, visual inspection detected visible particles in 100% of the samples. For all samples an increase in the number of fluffs is observed over time.

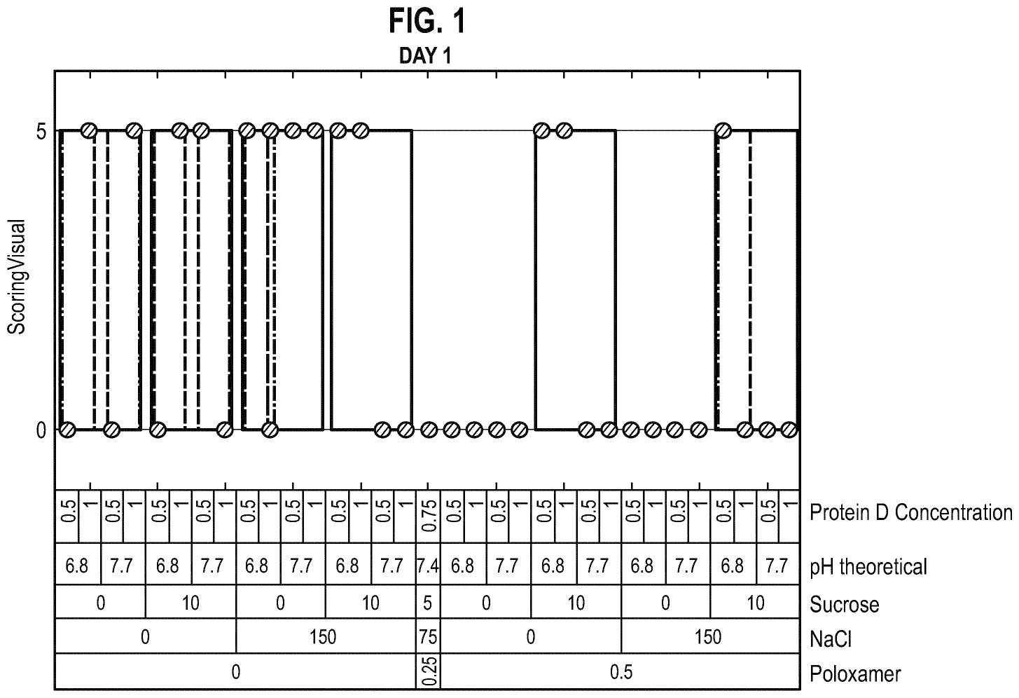

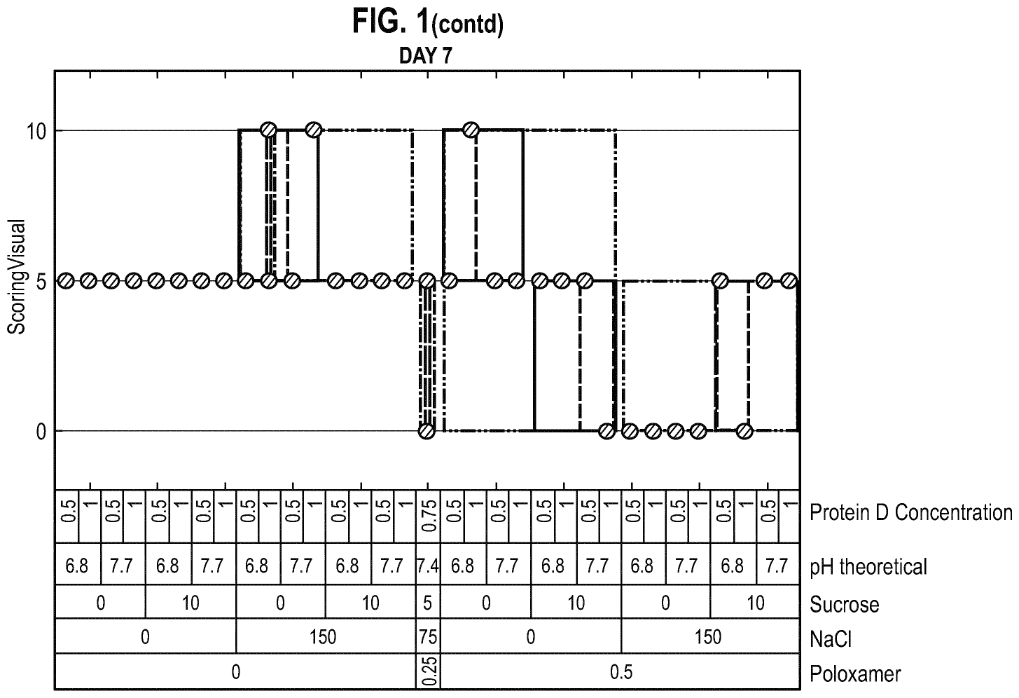

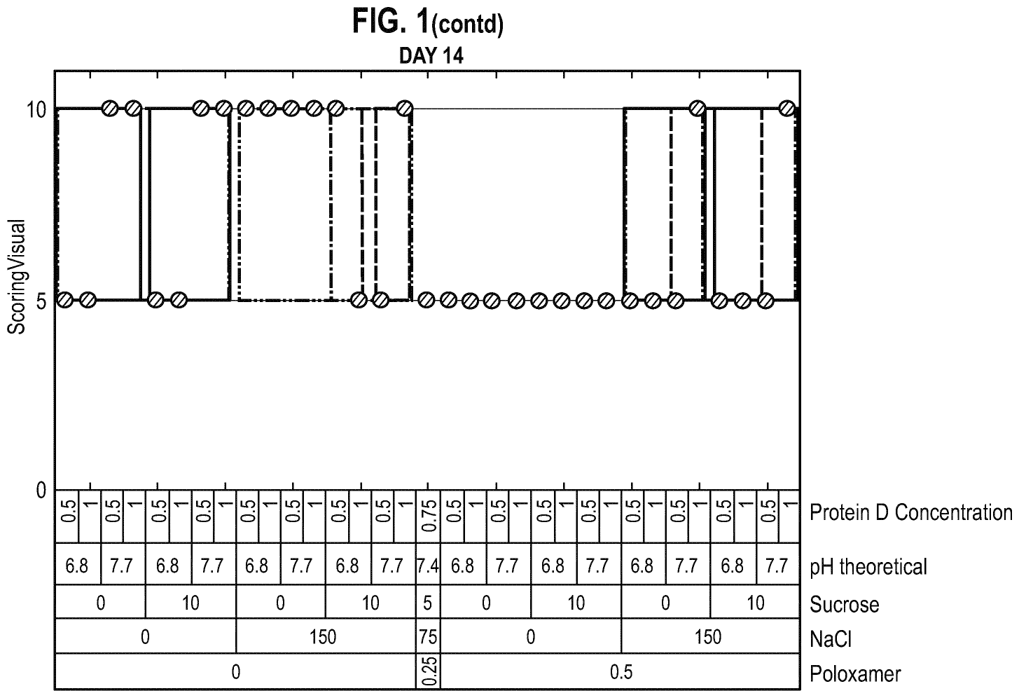

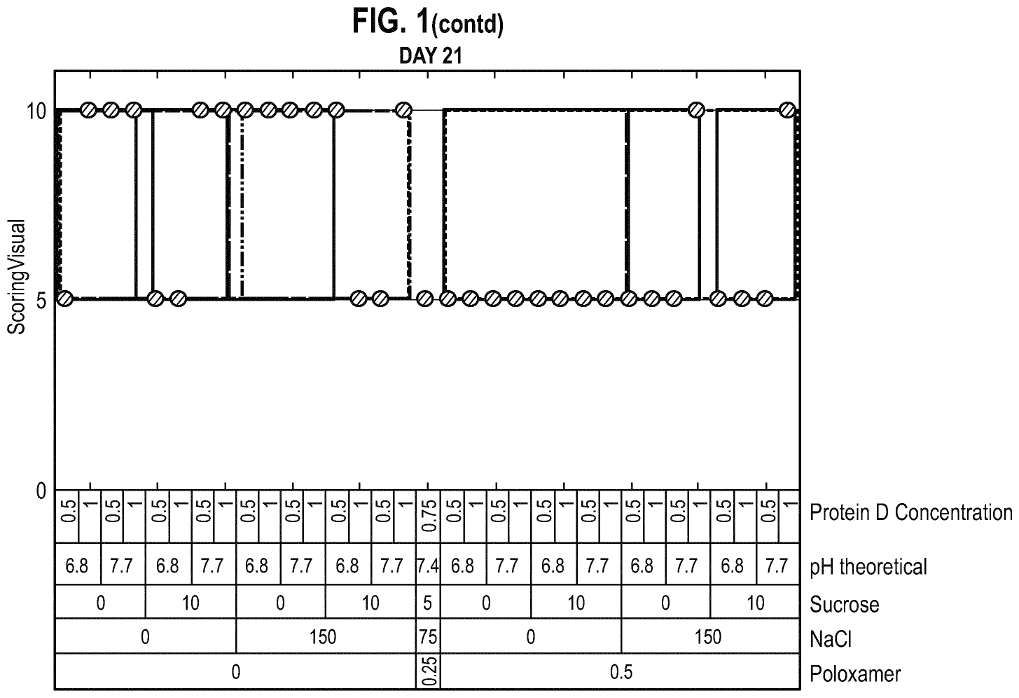

A statistical analysis was performed and the visual inspections were ranked (−=0; +=5 and ++=10). Based on this ranking the visual inspections were depicted (see FIG. 1). This statistical analysis was performed in order to confirm the visual observations. As visible on the FIG. 1 below, values on the left of the graph (samples without Poloxamer 188) were always greater than values on the right (samples containing 0.5% of Poloxamer 188). Based on the current visual inspection results the Poloxamer 188 seems to have an impact. There was no clear evidence of sucrose or NaCl impact on the visual inspections.

### Light Obscuration

The Light Obscuration measurements were performed at each time point (T 1 day, 7 days, 14 days & 21 days). After having generated data, two decisions were taken for statistical analysis of the data. The first one was to only take into account particles bigger than 35 μm (Particles are visible to the unaided eye from 50 μm). The second one was to sum the visible particles. This decision contributes to normalize the data.

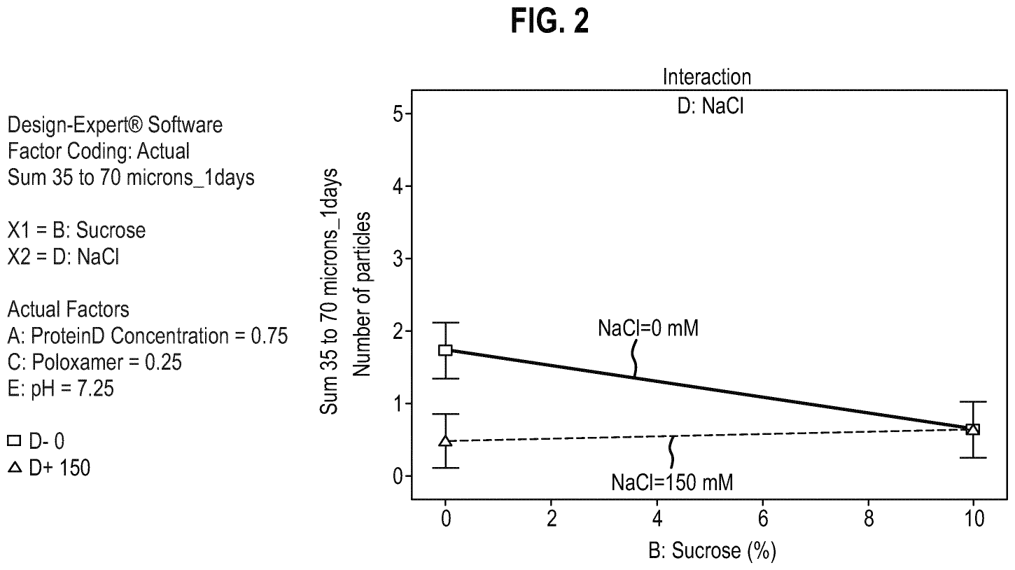

At day 1, a significant effect was observed for the Sucrose alone and NaCl alone (see FIG. 2): less visible particles were observed with addition of sucrose (with or without NaC) or with addition of NaCl, compared to without NaCl and without Sucrose. The presence of NaCl in addition of Sucrose didn't bring a greater impact on the decreasing of visible particles.

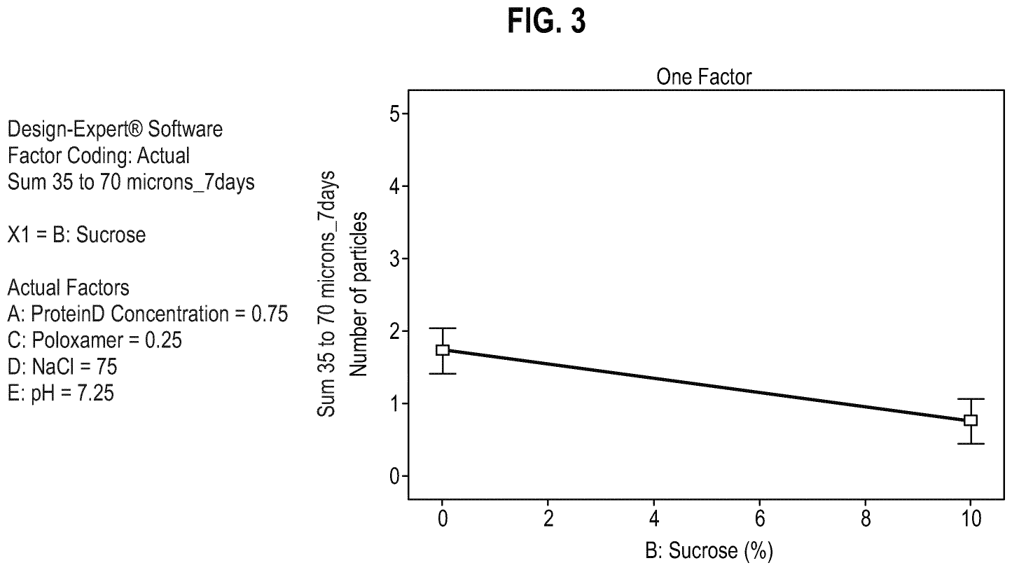

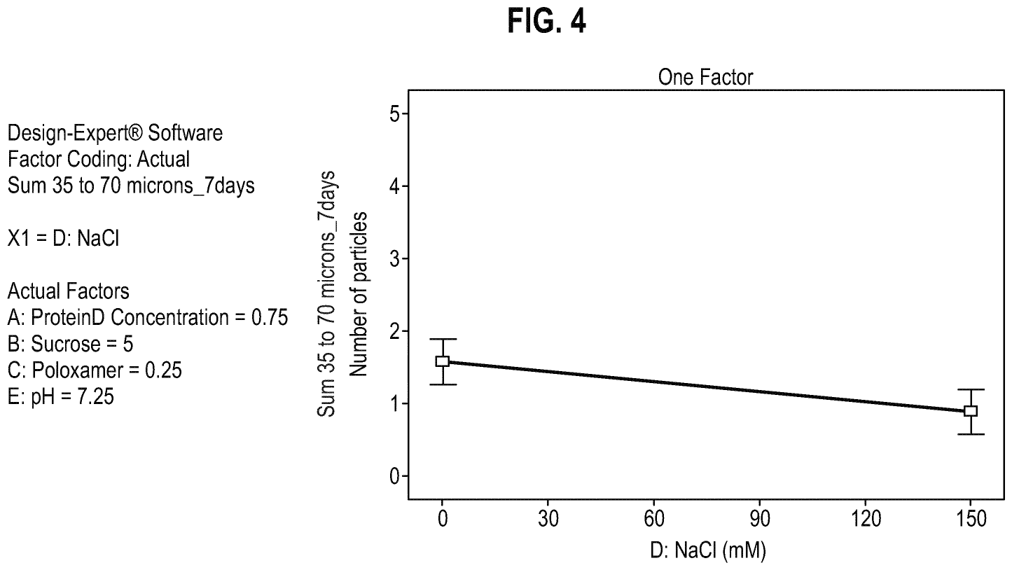

At day 7, a significant effect was observed for Sucrose and NaCl (see FIG. 3 & FIG. 4). For both, less visible particles were observed in their presence.

### Conclusions:

This evaluation demonstrated that:
Addition of NaCl was favourable according to Light obscuration results for the visible particles (based on the sum of 35 to 70 microns).
Addition of 10% m/v of sucrose was favourable according to Light obscuration results for the visible particles (based on the sum of 35 to 70 microns).
No effect was observed between 0.5 and 1 mg/ml for the Protein D concentration.
For Poloxamer 188 between 0% m/v and 0.5% m/v an impact was observed on the subvisible particles (lower than 25 microns) but not on the visible particles from the light obscuration results. However, the Poloxamer 188 may have an impact from the visual inspections.
No lessons could be learned for the pH due to a difference between the theoretical and the measured pH.

### Example 2: Screening of Excipients and their Impact on Particle Formation During Liquid Storage (Part 2)

In this study, the following parameters were studied on 2 batches of Protein D:
pH (2 levels)
6.4
7.4
Sucrose (2 levels)
10% m/v sucrose
20% m/v sucrose
Poloxamer 188 (2 levels)
0%
1%
NaCl (1 level)
150 mM
Protein D concentration (1 level)
1 mg/ml

The two frozen Protein D batches were thawed at 25° C. (air) in an incubator (1 h 30). After thawing, the Protein D was diluted to 20 mg/ml in 150 mM NaCl and then filtered on a 0.22 μm Millipore Millex™ Sterile Syringe Filter (SLGV033RS). Afterwards, the 28 conditions were formulated by Tecan®, through the use of a Tecan robot.

TABLE 2
DoE full factorial with in addition the 6 face centered points and 2 times the
current process (1 mg/ml PD in 150 mM NaCl)

| | Protein D | NaCl | Protein D | Sucrose | Poloxamer 188 | |
|---|---|---|---|---|---|---|
| #Samples | Batch | (mM) | (mg/ml) | (%) | (%) | Target pH |
| 18C0P02017 | APDOAPA024 | 150 | 1 | 0 | 0 | 6.80 |
| 18C0P02014 | APDOAPA024 | 150 | 1 | 10 | 0 | 6.40 |
| 18C0P02013 | APDOAPA024 | 150 | 1 | 10 | 0 | 7.40 |
| 18C0P02001 | APDOAPA024 | 150 | 1 | 10 | 0.5 | 6.90 |
| 18C0P02002 | APDOAPA024 | 150 | 1 | 10 | 1 | 6.40 |
| 18C0P02006 | APDOAPA024 | 150 | 1 | 10 | 1 | 7.40 |
| 18C0P02007 | APDOAPA024 | 150 | 1 | 15 | 0 | 6.90 |
| 18C0P02009 | APDOAPA024 | 150 | 1 | 15 | 0.5 | 6.40 |
| 18C0P02012 | APDOAPA024 | 150 | 1 | 15 | 0.5 | 6.90 |
| 18C0P02015 | APDOAPA024 | 150 | 1 | 15 | 0.5 | 6.90 |
| 18C0P02016 | APDOAPA024 | 150 | 1 | 15 | 0.5 | 7.40 |
| 18C0P02004 | APDOAPA024 | 150 | 1 | 15 | 1 | 6.90 |
| 18C0P02003 | APDOAPA024 | 150 | 1 | 20 | 0 | 6.40 |
| 18C0P02010 | APDOAPA024 | 150 | 1 | 20 | 0 | 7.40 |
| 18C0P02011 | APDOAPA024 | 150 | 1 | 20 | 0.5 | 6.90 |
| 18C0P02008 | APDOAPA024 | 150 | 1 | 20 | 1 | 6.40 |
| 18C0P02005 | APDOAPA024 | 150 | 1 | 20 | 1 | 7.40 |
| 18C0P02028 | APDOAPA023 | 150 | 1 | 0 | 0 | 6.80 |
| 18C0P02020 | APDOAPA023 | 150 | 1 | 10 | 0 | 6.40 |
| 18C0P02021 | APDOAPA023 | 150 | 1 | 10 | 0 | 7.40 |
| 18C0P02022 | APDOAPA023 | 150 | 1 | 10 | 1 | 6.40 |
| 18C0P02024 | APDOAPA023 | 150 | 1 | 10 | 1 | 7.40 |
| 18C0P02018 | APDOAPA023 | 150 | 1 | 15 | 0.5 | 6.90 |
| 18C0P02027 | APDOAPA023 | 150 | 1 | 15 | 0.5 | 6.90 |
| 18C0P02023 | APDOAPA023 | 150 | 1 | 20 | 0 | 6.40 |
| 18C0P02019 | APDOAPA023 | 150 | 1 | 20 | 0 | 7.40 |
| 18C0P02026 | APDOAPA023 | 150 | 1 | 20 | 1 | 6.40 |
| 18C0P02025 | APDOAPA023 | 150 | 1 | 20 | 1 | 7.40 |

### Visual Inspection

All visual inspections were performed in a black and white visual inspection post (Using only the black background) by five persons for time point 1 day and by seven persons for time points 7 & 14 days. All samples were classified using a graduation with 5 levels (0, −, +, ++& +++). Respectively for no particles, a few particles, some particles, a lot of particles and plenty of particles. A statistical analysis was performed and the visual inspections were ranked (0=0, −=1, +=2, ++=3 and +++=4).

As for Example 1 visible particles were present in some samples already after 24 h of storage at 2/8° C. For all samples an increase in the number of visible particles was observed over time. A statistical analysis was performed and the visual inspections were ranked (0=0, −=1, +=2, ++=3 and +++=4). Based on this ranking the visual inspections were depicted. This quotation was then treated in a statistical analysis in order to confirm the visual observations. Results have been ordered sorting first by the Poloxamer 188, the sucrose or the pH at time points 1, 7 and 14 days.

Considering the average of the scores from all the observers at day 1, 7 and 14, a significant effect was observed for Poloxamer 188 with lower (i.e. reduction of the visible particles) scores in presence of Poloxamer 188.

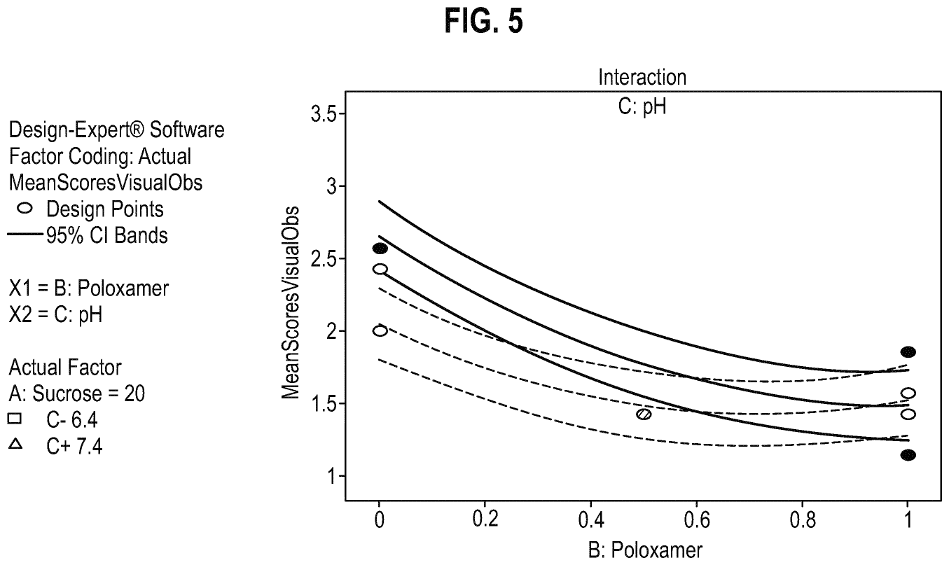

Considering the average of the scores from all the observers, a trend was observed with lower (i.e. reduction of the visible particles) scores when pH increases. But only at day 7 a significant effect was observed (p-value=0.0129) with lower scores at pH 6.9. At day 7, a significant interaction between Poloxamer 188 and pH was also observed (see FIG. 5). Indeed, when there was no Poloxamer 188, the pH had an important effect. The number of visible particles decreased with a higher pH.

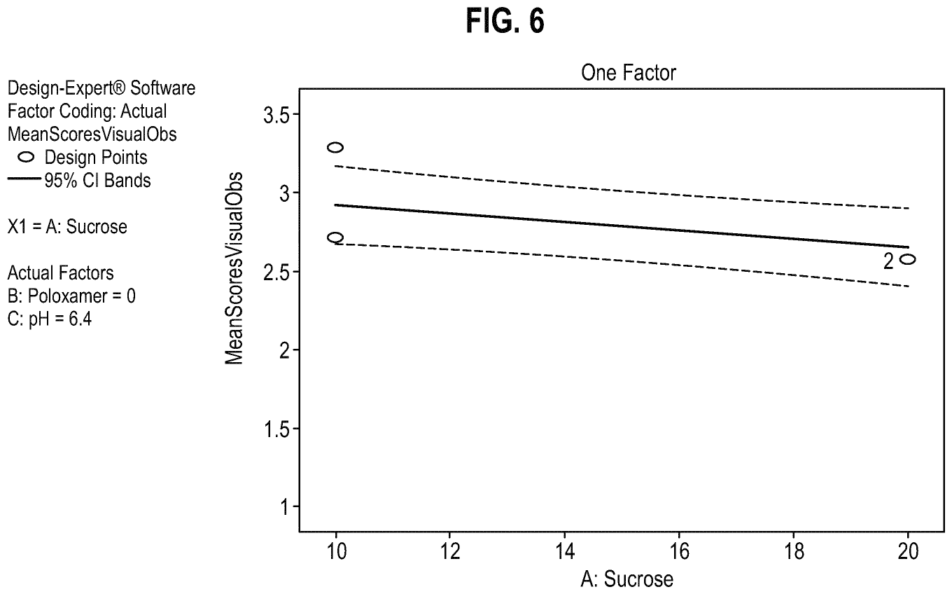

Considering the average of the scores from al the observers at day 1, no significant effect was observed. At day 7 a slight but significant effect (p-value=0.0179) was observed for sucrose (see FIG. 6) with lower (i.e. reduction of the visible particles) scores in presence of 20% of sucrose. At day 14, an effect (p-value=0.08) was observed for sucrose with lower (i.e. reduction of the visible particles) scores in presence of 20% of sucrose.

By considering the three time points (day 1, 7 and 14), the presence of Poloxamer 188 was favourable to reduce the number of visible particles. This reduction might be slightly improved with the highest level of sucrose (20% m/v) (see FIG. 5). But although a statistical relevant effect was observed for the sucrose, the practical relevance was considered limited.

### Light Obscuration

Light Obscuration measurements were performed at each time point (T 1 day, 7 days & 14 days). Only particles bigger or equal than 35 μm have been taken into account (Particles are visible to the unaided eye from 50 μm). A statistical analysis was performed based on the sum of the particles between 35 μm and 70 μm.

The sample 18COP02003 (no Poloxamer, pH at 6.4; sucrose at 20% m/v) was detected atypical over the entire range of particles (from 2 to 125 μm). No cause was identified to explain this atypical result.

A statistical analysis was performed. Results were analysed sorting by the Poloxamer 188, the sucrose or the pH at time point 1, 7 and 14 days. Results were analysed based on the average of the measurements. Each Light Obscuration measurement was obtained by analysing four times 1 millilitre of product. The first value, obtained on the first millilitre was discarded and only served to flush the equipment.

Considering the average of the 3 measurements for the sum of particles between 35 microns and 70 microns at day 1, 7 and 14, for each configuration tested, the number of particles was lower in presence of Poloxamer. This was also the case when removing the atypical result (configuration: no Poloxamer, pH at 6.4, and sucrose at 20% m/v).

### Conclusions:

This evaluation demonstrated that:
Addition of Poloxamer 188 had a significant effect in the reduction of the visible particles whether by Light Obscuration or by Visual Inspection or by Occhio. This was in line with what was observed in Example 1 up to 0.5% m/v. The observed reduction was almost similar at 1% m/v and at 0.5% m/v.
The sucrose increase from 10% m/v to 20% m/v didn't have a practical significant impact in the reduction of the visible particles. The sucrose increase allowed rising the temperature of melting and the onset aggregation temperature. From a visual inspection point of view, this increase slightly improved the reduction of the visible particles, but this observation was not correlated with the Light Obscuration for which 10% m/v sucrose was favourable.

### Example 3: Optimized Process for Thawing, Dilution & Filtration of Protein D

For the first step, the Protein D (4.5 ml Nunc container) was thawed statically at 25° C. in an incubator. Once thawed, the Protein D was homogenized by stirring with a magnetic bar. Subsequently the Protein D was diluted in a Duran Schott glass container to img/ml in 150 mM NaCl, 10% w/v Sucrose, 1% w/v Poloxamer 188, 12.5 mM PO₄³⁻ KH₂PO₄/K₂HPO₄ Phosphate buffer, pH 6.8 following the flow sheet below (FIG. 1). The addition was done by pipette or graduated cylinder glass. To reach these target concentrations, a 15.75% w/v sucrose solution, a 100 mM K₂PO₄/KH₂HPO₄ 1160 mM NaCl pH 6.9 buffer and a 10% w/v Poloxamer 188 solution was used. The Protein D dilution was based on the Protein D content by RP-UPLC previously obtained on other aliquots of the same three drug substance batches. Once diluted, the Protein D was filtered by using an OptiScale® 47 filter (0.22 μm Durapore® PVDF membrane 17.7 cm²-Polypropylene cartridge) and a peristaltic pump (flow rate 0.7 ml/min/cm²).

### Example 4: Comparison of Protein D Dilution Processes

| Reference Process | Optimized Process |
|---|---|
| 1 mg/mL Protein D | 1 mg/mL Protein D |
| 150 mM NaCl | 150 mM NaCl |
| | 10% w/v sucrose |
| | 1% w/v PX188 |
| | 12.5 mM K/K₂PO₄ |
| | pH 6.8 |

### Optimized Process:

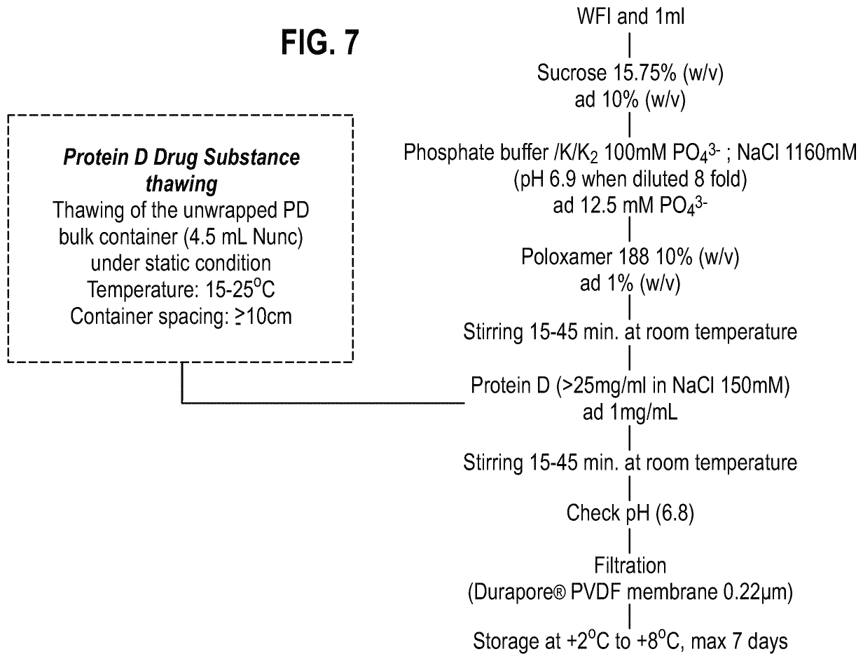

Protein D dilution was carried out according to the process provided in the flow sheet according to Example 3 and FIG. 7 (Optimized Process).

### Reference Process:

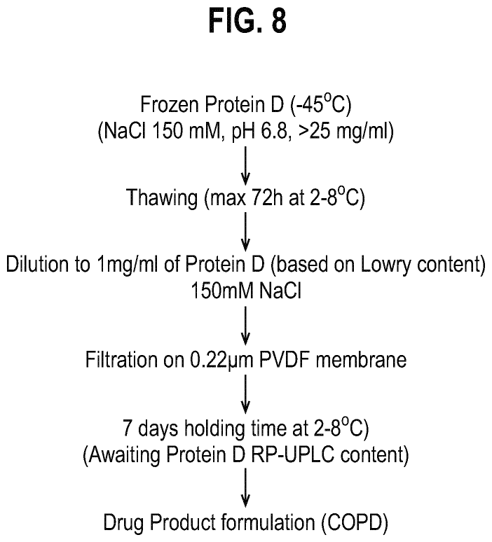

Protein D dilution was carried out according to the process provided in the flow sheet according to FIG. 8 (Reference Process). The frozen PD Drug Substance (stored at 45° C., pH 6.8) was thawed as follows:
Aliquots of 2-4 g: min7 h-max72 h at 2-8° C. or min1 h-max2 h at 25±1° C. (Water-bath)
Aliquots of 18 g: min24 h-max72 h at 2-8° C. or min2 h-max3 h at 25±1° C. (Water-bath)
   Once thawed, PD was diluted to −1 mg/ml with NaCl 150 mM and filtered on 0.22 μm. Filter characteristics: Millex (0.45-) 0.22 μm PVDF, optimal protein load-to-area ratio: 90 mg prot/cm² (eg. 20 ml PD at 20 mg/ml filtered on Millex GV 33 mm 0.22 μm 4.5 cm³).

Three different Protein D Drug Substance batches (APDOAPA024, APDOBPA027 & APDOBPA029) were evaluated. For each batch, eight dilutions of Protein D at 1 mg/ml were performed: four times the optimized configurations (10% m/v Sucrose, 1% m/v Poloxamer 188, 150 mM NaCl, 1 mg/ml Protein D, 12.5 mM Phosphate buffer K₂HPO₄/KH₂PO₄, pH 6.8) and four times the current process as reference (NaCl 150 mM, pH 6.8). The targeted Protein D concentration of img/ml was based on Lowry value.

The Optimized and Reference protein D dilution processes were compared using the following analytical techniques (as described above):
Particles detection by Light Obscuration, Occhio (Flow cam) & Visual Inspection
Secondary & Tertiary Structure by intrinsic fluorescence, FTIR & Far-UV Circular Dichroism
Protein D content by RP-UPLC.

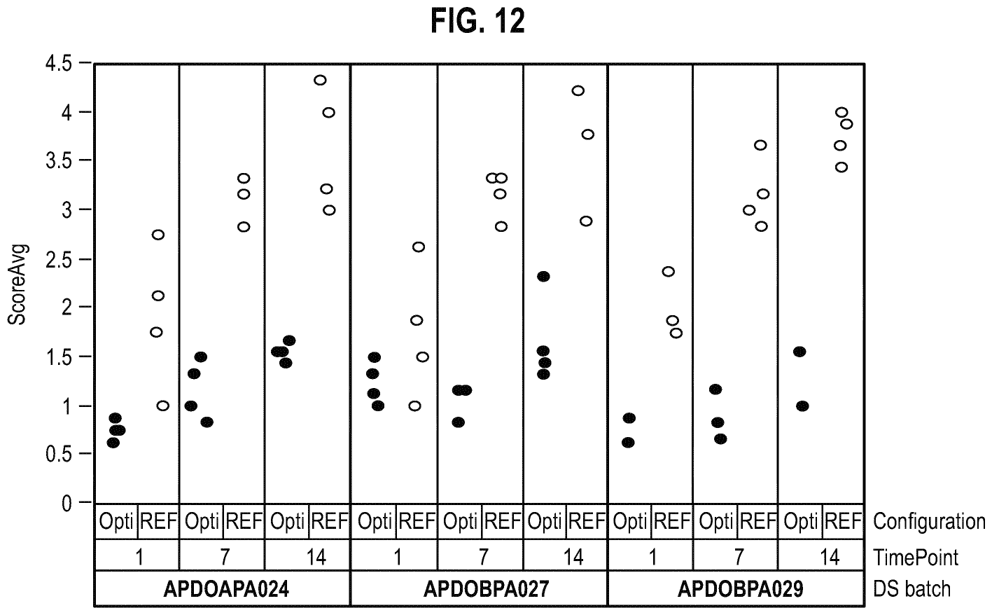

All visual inspections were performed in a black and white visual inspection post (using only the black background) by eleven persons but not by all of them for each time point. All samples were classified giving a score from 0 (no particle) to 6 (full of visible particles). Only figures with the of the average scores at the three-time points (day 1, 7 and 14) of all observers is shown in FIG. 12.

A multivariate analysis was carried out using the PCA method (Principal Components Analysis). Multivariate analysis is intended to synthesize information from several variables into two dimensions, to better explain it.

### Results:

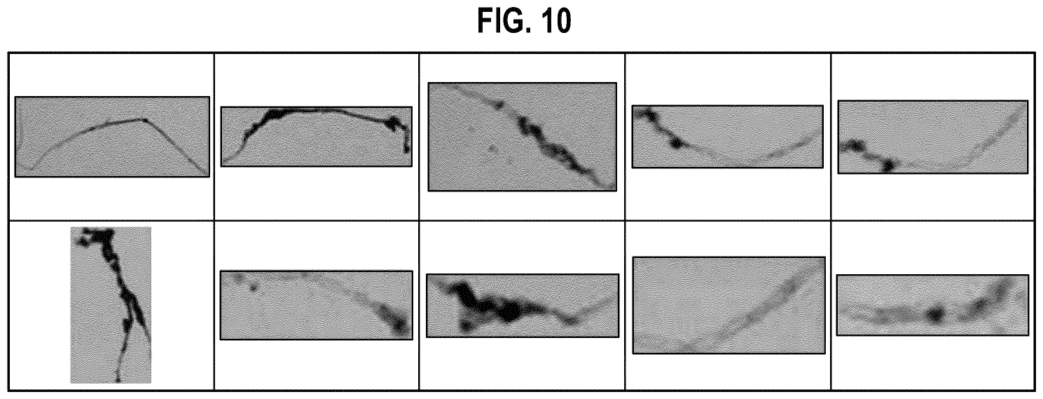

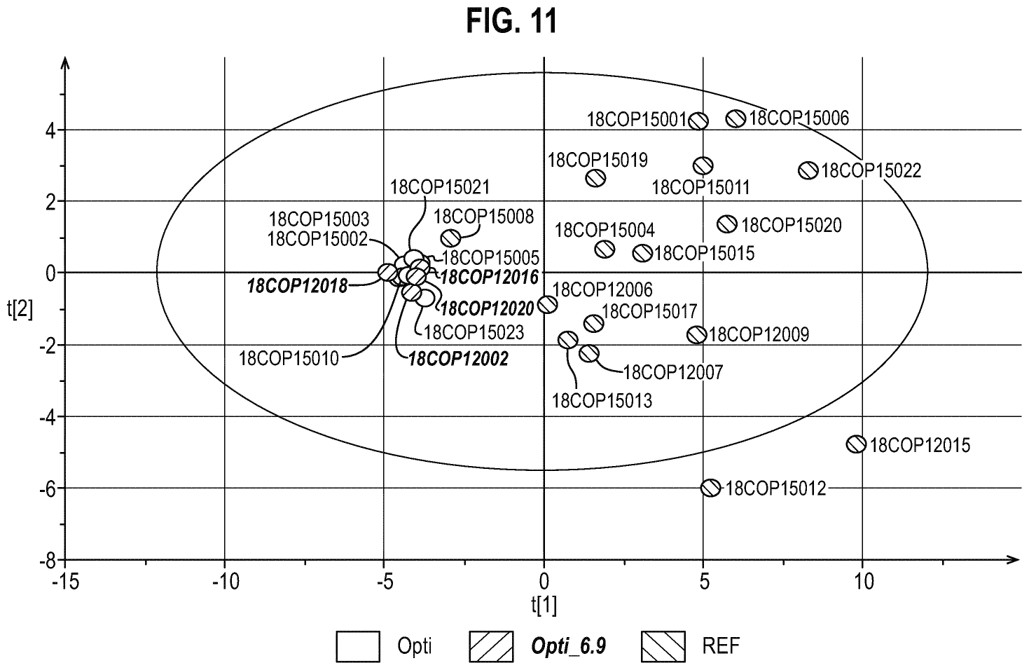

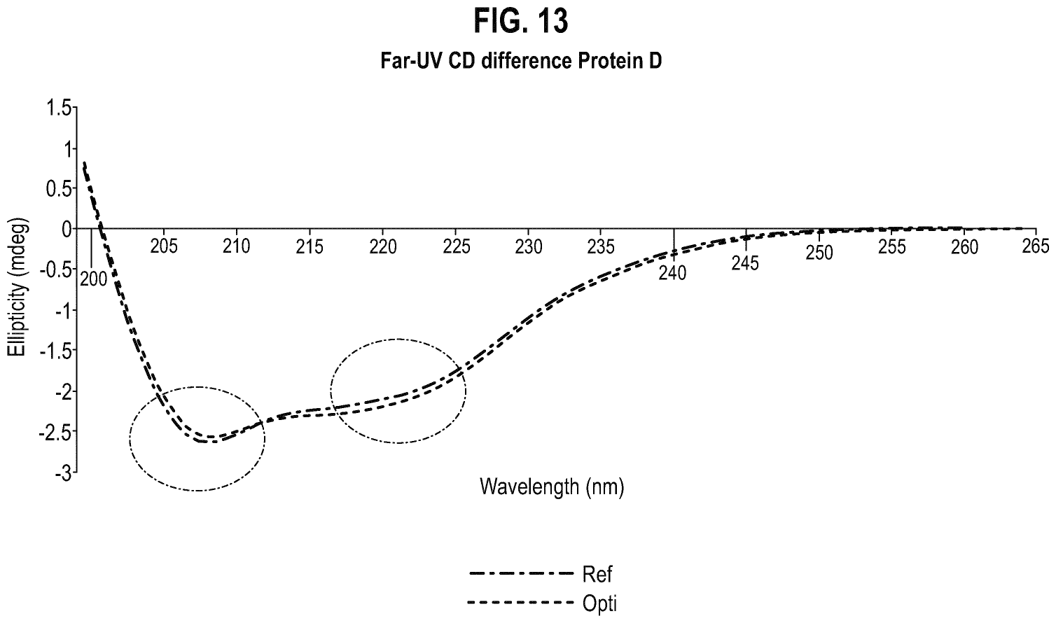

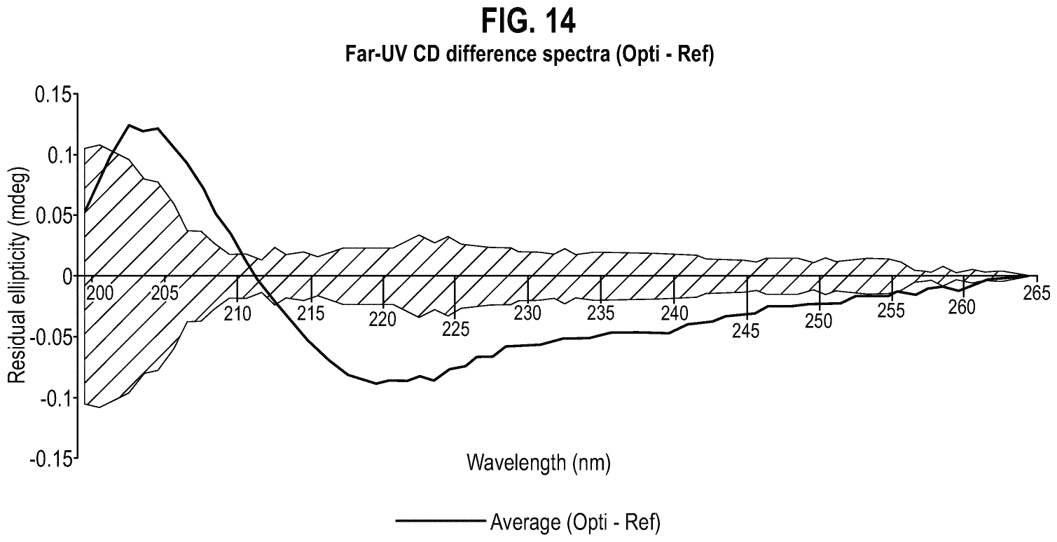

FIG. 9 represents the sum of particles from 50 to 1000 μm detected by Occhio at 3 time points (1, 7 & 14 days) for the optimized liquid composition & reference samples. A clear evolution in the number of particles was observed for the reference process, the number remained more stable for the optimized composition.
FIG. 10 provides examples of the pictures of visible particles captured by Occhio on a Protein D reference sample (1 mg/ml in 150 mM NaC)
FIG. 11 represents the multivariate analysis (PCA) considering the entire range of the Light Obscuration and Occhio measurements. A clear discrimination is observed between the optimized and the reference samples. Optimized samples were more homogeneous than reference samples. The horizontal axis summarizes the number of particles over the entire range: more particles were measured for the reference samples over the entire range of measurement for both Light Obscuration and Occhio. The vertical axis is more discriminating for the reference samples (for the optimized samples, no spread over the vertical axis was observed). The samples at the top are characterized by a higher number of visible particles and lower number of subvisible particles. It can be inferred that the optimized process is more reproducible.
FIG. 12 represents the average scores from the observers having performed the visual inspection in a black & white post on 3 different lots. Scores are lower for optimized samples whatever the day & the Protein D batch.
FIGS. 13 and 14 represent Far-UV CD spectra and the difference spectrum showing slight differences in 208 nm and 222 nm regions. This reflects a slight modification of secondary structure (Increase of α-helix content).

### Conclusions:

All evaluations carried out on Protein D demonstrated:

A significant reduction of the number of visible particles when adding to the liquid Protein D:

### 1% w/v Poloxamer 188

### 150 mM NaCl

### 10% w/v Sucrose

12.5 mM K₂HPO₄/KH₂PO₄ buffer pH 6.8
No impact on the Protein D profile, size and Molar mass
No major impact on the PD content & antigenicity
Slight differences on the secondary and tertiary structure (Protein D is slightly more folded in this optimised composition).

Moreover, filtrations performed on Protein D after dilution with the new composition show no content loss.

### Sequences:

SEQ ID NO 1: Protein D (364 amino acids)
MetLysLeuLysThrLeuAlaLeuSerLeuLeuAlaAlaGlyValLeuAlaGly
CysSerSerHisSerSerAsnMetAlaAsnThrGlnMetLysSerAspLysIle
IleIleAlaHisArgGlyAlaSerGlyTyrLeuProGluHisThrLeuGluSerLysAla
LeuAlaPheAlaGlnGlnAlaAspTyrLeuGluGlnAspLeuAlaMetThrLysAspGly
ArgLeuValValIleHisAspHisPheLeuAspGlyLeuThrAspValAlaLysLysPhe
ProHisArgHisArgLysAspGlyArgTyrTyrValIleAspPheThrLeuLysGluIle
GlnSerLeuGluMetThrGluAsnPheGluThrLysAspGlyLysGlnAlaGlnValTyr
ProAsnArgPheProLeuTrpLysSerHisPheArgIleHisThrPheGluAspGluIle
GluPheIleGlnGlyLeuGluLysSerThrGlyLysLysValGlyIleTyrProGluIle
LysAlaProTrpPheHisHisGlnAsnGlyLysAspIleAlaAlaGluThrLeuLysVal
LeuLysLysTyrGlyTyrAspLysLysThrAspMetValTyrLeuGlnThrPheAspPhe
AsnGluLeuLysArgIleLysThrGluLeuLeuProGlnMetGlyMetAspLeuLysLeu
ValGlnLeuIleAlaTyrThrAspTrpLysGluThrGlnGluLysAspProLysGlyTyr
TrpValAsnTyrAsnTyrAspTrpMetPheLysProGlyAlaMetAlaGluValValLys
TyrAlaAspGlyValGlyProGlyTrpTyrMetLeuValAsnLysGluGluSerLysPro
AspAsnIleValTyrThrProLeuValLysGluLeuAlaGlnTyrAsnValGluValHis
ProTyrThrValArgLysAspAlaLeuProGluPhePheThrAspValAsnGlnMetTyr
AspAlaLeuLeuAsnLysSerGlyAlaThrGlyValPheThrAspPheProAspThrGly
ValGluPheLeuLysGlyIleLys
SEQ ID NO: 2: Protein D fragment with MDP tripeptide from NS1
(348 amino acids)
MetAspProSerSerHisSerSerAsnMetAlaAsnThrGlnMetLysSerAspLysIle
IleIleAlaHisArgGlyAlaSerGlyTyrLeuProGluHisThrLeuGluSerLysAla
LeuAlaPheAlaGlnGlnAlaAspTyrLeuGluGlnAspLeuAlaMetThrLysAspGly
ArgLeuValValIleHisAspHisPheLeuAspGlyLeuThrAspValAlaLysLysPhe
ProHisArgHisArgLysAspGlyArgTyrTyrValIleAspPheThrLeuLysGluIle
GlnSerLeuGluMetThrGluAsnPheGluThrLysAspGlyLysGlnAlaGlnValTyr
ProAsnArgPheProLeuTrpLysSerHisPheArgIleHisThrPheGluAspGluIle
GluPheIleGlnGlyLeuGluLysSerThrGlyLysLysValGlyIleTyrProGluIle
LysAlaProTrpPheHisHisGlnAsnGlyLysAspIleAlaAlaGluThrLeuLysVal
LeuLysLysTyrGlyTyrAspLysLysThrAspMetValTyrLeuGlnThrPheAspPhe
AsnGluLeuLysArgIleLysThrGluLeuLeuProGlnMetGlyMetAspLeuLysLeu
ValGlnLeuIleAlaTyrThrAspTrpLysGluThrGlnGluLysAspProLysGlyTyr
TrpValAsnTyrAsnTyrAspTrpMetPheLysProGlyAlaMetAlaGluValValLys
TyrAlaAspGlyValGlyProGlyTrpTyrMetLeuValAsnLysGluGluSerLysPro
AspAsnIleValTyrThrProLeuValLysGluLeuAlaGlnTyrAsnValGluValHis
ProTyrThrValArgLysAspAlaLeuProGluPhePheThrAspValAsnGlnMetTyr
AspAlaLeuLeuAsnLysSerGlyAlaThrGlyValPheThrAspPheProAspThrGly
ValGluPheLeuLysGlyIleLys
SEQ ID NO: 3:
SerSerHisSerSerAsnMetAlaAsnThr
SEQ ID NO: 4: Protein E from H. influenzae
MKKIILTLSL GLLTACSAQI QKAEQNDVKL APPTDVRSGY IRLVKNVNYY IDSESIWVDN
QEPQIVHFDA VVNLDKGLYV YPEPKRYARS VRQYKILNCA NYHLTQVRTD FYDEFWGQGL
RAAPKKQKKH TLSLTPDTTL YNAAQIICAN YGEAFSVDKK
SEQ ID NO: 5: Amino acids 20-160 of Protein E
I QKAEQNDVKL APPTDVRSGY IRLVKNVNYY IDSESIWVDN QEPQIVHFDA VVNLDKGLYV
YPEPKRYARS VRQYKILNCA NYHLTQVRTD FYDEFWGQGL RAAPKKQKKH TLSLTPDTTL
YNAAQIICAN YGEAFSVDKK
SEQ ID NO: 6 PilA from H. influenzae
MKLTTQQTLK KGFTLIELMI VIAIIAILAT IAIPSYQNYT KKAAVSELLQ ASAPYKADVE
LCVYSTNETT NCTGGKNGIA ADITTAKGYV KSVTTSNGAI TVKGDGTLAN MEYILQATGN
AATGVTWTTT CKGTDASLFP ANFCGSVTQ
SEQ ID NO: 7 Amino acids 40-149 of PilA from H. influenzae strain
86-028NP
T KKAAVSELLQ ASAPYKADVE LCVYSTNETT NCTGGKNGIA ADITTAKGYV KSVTTSNGAI
TVKGDGTLAN MEYILQATGN AATGVTWTTT CKGTDASLFP ANFCGSVTQ
SEQ ID NO: 8: LVL735 (protein): (pelB sp)(ProtE aa 20-160)(GG)
(PilA aa40-149)
MKYLLPTAAA GLLLLAAQPA MAIQKAEQND VKLAPPTDVR SGYIRLVKNV NYYIDSESIW
VDNQEPQIVH FDAVVNLDKG LYVYPEPKRY ARSVRQYKIL NCANYHLTQV RTDFYDEFWG
QGLRAAPKKQ KKHTLSLTPD TTLYNAAQII CANYGEAFSV DKKGGTKKAA VSELLQASAP
YKADVELCVY STNETTNCTG GKNGIAADIT TAKGYVKSVT TSNGAITVKG DGTLANMEYI
LQATGNAATG VTWTTTCKGT DASLFPANFC GSVTQ
SEQ ID NO: 9: PE-PilA fusion protein without signal peptide
IQKAEQND VKLAPPTDVR SGYIRLVKNV NYYIDSESIW VDNQEPQIVH FDAVVNLDKG
LYVYPEPKRY ARSVRQYKIL NCANYHLTQV RTDFYDEFWG QGLRAAPKKQ KKHTLSLTPD
TTLYNAAQII CANYGEAFSV DKKGGTKKAA VSELLQASAP YKADVELCVY STNETTNCTG
GKNGIAADIT TAKGYVKSVT TSNGAITVKG DGTLANMEYI LQATGNAATG VTWTTTCKGT
DASLFPANFC GSVTQ
SEQ ID NO: 10: UspA2 from ATCC 25238
MKTMKLLPLKIAVTSAMIIGLGAASTANAQAKNDITLEDLPYLIKKIDQNELEADIGDIT
ALEKYLALSQYGNILALEELNKALEELDEDVGWNQNDIANLEDDVETLTKNQNALAEQGE
AIKEDLQGLADFVEGQEGKILQNETSIKKNTQRNLVNGFEIEKNKDAIAKNNESIEDLYD
FGHEVAESIGEIHAHNEAQNETLKGLITNSIENTNNITKNKADIQALENNVVEELFNLSG
RLIDQKADIDNNINNIYELAQQQDQHSSDIKTLKKNVEEGLLELSGHLIDQKTDIAQNQA
NIQDLATYNELQDQYAQKQTEAIDALNKASSENTQNIEDLAAYNELQDAYAKQQTEAIDA
LNKASSENTQNIEDLAAYNELQDAYAKQQTEAIDALNKASSENTQNIAKNQADIANNINN
IYELAQQQDQHSSDIKTLAKASAANTDRIAKNKADADASFETLTKNQNTLIEKDKEHDKL
ITANKTAIDANKASADTKFAATADAITKNGNAITKNAKSITDLGTKVDGFDSRVTALDTK
VNAFDGRITALDSKVENGMAAQAALSGLFQPYSVGKFNATAALGGYGSKSAVAIGAGYRV
NPNLAFKAGAAINTSGNKKGSYNIGVNYEF
SEQ ID NO: 11: MC-001 (protein) - (M)(UspA2 amino acids 30-540)
(ASHHHHHH)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAI
AKNNESIEDLYDFGHEVAESIGEIHANNEAQNETLKGLITNSIENTNNITKNKADIQALENNVVEELFNLS
GRLIDQKADIDNNINNIYELAQQQDQHSSDIKTLKKNVEEGLLELSGHLIDQKTDIAQNQANIQDLATYN
ELQDQYAQKQTEAIDALNKASSENTQNIEDLAAYNELQDAYAKQQTEAIDALNKASSENTQNIEDLAA
YNELQDAYAKQQTEAIDALNKASSENTQNIAKNQADIANNINNIYELAQQQDQHSSDIKTLAKASAANT
DRIAKNKADADASFETLTKNQNTLIEKDKEHDKLITANKTAIDANKASADTKFAATADAITKNGNAITKNA
KSITDLGTKVDGFDSRVTALDTKASHHHHHH
SEQ ID NO: 12 MC-002 (Protein) - (M)(UspA2 amino acids 30-540)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAI
AKNNESIEDLYDFGHEVAESIGEIHAHNEAQNETLKGLITNSIENTNNITKNKADIQALENNVVEELFNLS
GRLIDQKADIDNNINNIYELAQQQDQHSSDIKTLKKNVEEGLLELSGHLIDQKTDIAQNQANIQDLATYN
ELQDQYAQKQTEAIDALNKASSENTQNIEDLAAYNELQD
AYAKQQTEAIDALNKASSENTQNIEDLAAYNELQDAYAKQQTEAIDALNKASSENTQNIAKNQADIANN
INNIYELAQQQDQHSSDIKTLAKASAANTDRIAKNKADADASFETLTKNQNTLIEKDKEHDKLITANKTAI
DANKASADTKFAATADAITKNGNAITKNAKSITDLGTKVDGFDSRVTALDTK
SEQ ID NO: 13 MC-003 (Protein) - (M)(UspA2 amino acids 30-540)(H)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAI
AKNNESIEDLYDFGHEVAESIGEIHAHNEAQNETLKGLITNSIENTNNITKNKADIQALENNVVEELFNLS
GRLIDQKADIDNNINNIYELAQQQDQHSSDIKTLKKNVEEGLLELSGHLIDQKTDIAQNQANIQDLATYN
ELQDQYAQKQTEAIDALNKASSENTQNIEDLAAYNELQDAYAKQQTEAIDALNKASSENTQNIEDLAA
YNELQDAYAKQQTEAIDALNKASSENTQNIAKNQADIANNINNIYELAQQQDQHSSDIKTLAKASAANT
DRIAKNKADADASFETLTKNQNTLIEKDKEHDKLITANKTAIDANKASADTKFAATADAITKNGNAITKNA
KSITDLGTKVDGFDSRVTALDTKH
SEQ ID NO: 14 MC-004 (Protein) - (M)(UspA2 amino acids 30-540)(HH)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAI
AKNNESIEDLYDFGHEVAESIGEIHAHNEAQNETLKGLITNSIENTNNITKNKADIQALENNVVEELFNLS
GRLIDQKADIDNNINNIYELAQQQDQHSSDIKTLKKNVEEGLLELSGHLIDQKTDIAQNQANIQDLATYN
ELQDQYAQKQTEAIDALNKASSENTQNIEDLAAYNELQDAYAKQQTEAIDALNKASSENTQNIEDLAA
YNELQDAYAKQQTEAIDALNKASSENTQNIAKNQADIANNINNIYELAQQQDQHSSDIKTLAKASAANT
DRIAKNKADADASFETLTKNQNTLIEKDKEHDKLITANKTAIDANKASADTKFAATADAITKNGNAITKNA
KSITDLGTKVDGFDSRVTALDTKHH
SEQ ID NO: 15 MC-005 (Protein) - (M)(UspA2 amino acids 30-519)(ASHHHHHH)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAI
AKNNESIEDLYDFGHEVAESIGEIHAHNEAQNETLKGLITNSIENTNNITKNKADIQALENNVVEELFNLS
GRLIDQKADIDNNINNIYELAQQQDQHSSDIKTLKKNVEEGLLELSGHLIDQKTDIAQNQANIQDLATYN
ELQDQYAQKQTEAIDALNKASSENTQNIEDLAAYNELQDAYAKQQTEAIDALNKASSENTQNIEDLAA
YNELQDAYAKQQTEAIDALNKASSENTQNIAKNQADIANNINNIYELAQQQDQHSSDIKTLAKASAANT
DRIAKNKADADASFETLTKNQNTLIEKDKEHDKLITANKTAIDANKASADTKFAATADAITKNGNAITKNA
KSASHHHHHH
SEQ ID NO: 16 MC-006 (Protein) - (M)(UspA2 amino acids 30-519)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAI
AKNNESIEDLYDFGHEVAESIGEIHAHNEAQNETLKGLITNSIENTNNITKNKADIQALENNVVEELFNLS
GRLIDQKADIDNNINNIYELAQQQDQHSSDIKTLKKNVEEGLLELSGHLIDQKTDIAQNQANIQDLATYN
ELQDQYAQKQTEAIDALNKASSENTQNIEDLAAYNELQDAYAKQQTEAIDALNKASSENTQNIEDLAA
YNELQDAYAKQQTEAIDALNKASSENTQNIAKNQADIANNINNIYELAQQQDQHSSDIKTLAKASAANT
DRIAKNKADADASFETLTKNQNTLIEKDKEHDKLITANKTAIDANKASADTKFAATADAITKNGNAITKNA
KS
SEQ ID NO: 17 MC-007 (Protein) - (M)(UspA2 amino acids 30-564)(ASHHHHHH)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAI
AKNNESIEDLYDFGHEVAESIGEIHAHNEAQNETLKGLITNSIENTNNITKNKADIQALENNVVEELFNLS
GRLIDQKADIDNNINNIYELAQQQDQHSSDIKTLKKNVEEGLLELSGHLIDQKTDIAQNQANIQDLATYN
ELQDQYAQKQTEAIDALNKASSENTQNIEDLAAYNELQDAYAKQQTEAIDALNKASSENTQNIEDLAA
YNELQDAYAKQQTEAIDALNKASSENTQNIAKNQADIANNINNIYELAQQQDQHSSDIKTLAKASAANT
DRIAKNKADADASFETLTKNQNTLIEKDKEHDKLITANKTAIDANKASADTKFAATADAITKNGNAITKNA
KSITDLGTKVDGFDSRVTALDTKVNAFDGRITALDSKVENGMAAQAAASHHHHHH
SEQ ID NO: 18 MC-008 (Protein) - (M)(UspA2 30-564)(HH)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAI
AKNNESIEDLYDFGHEVAESIGEIHAHNEAQNETLKGLITNSIENTNNITKNKADIQALENNVVEELFNLS
GRLIDQKADIDNNINNIYELAQQQDQHSSDIKTLKKNVEEGLLELSGHLIDQKTDIAQNQANIQDLATYN
ELQDQYAQKQTEAIDALNKASSENTQNIEDLAAYNELQDAYAKQQTEAIDALNKASSENTQNIEDLAA
YNELQDAYAKQQTEAIDALNKASSENTQNIAKNQADIANNINNIYELAQQQDQHSSDIKTLAKASAANT
DRIAKNKADADASFETLTKNQNTLIEKDKEHDKLITANKTAIDANKASADTKFAATADAITKNGNAITKNA
KSITDLGTKVDGFDSRVTALDTKVNAFDGRITALDSKVENGMAAQAAHH
SEQ ID NO: 19 MC-009 (Protein) - (M)(UspA2 31-564)(HH)
MAKNDITLEDLPYLIKKIDQNELEADIGDITALEKYLALSQYGNILALEELNKALEELDEDVGWNQNDIAN
LEDDVETLTKNQNALAEQGEAIKEDLQGLADFVEGQEGKILQNETSIKKNTQRNLVNGFEIEKNKDAIA
KNNESIEDLYDFGHEVAESIGEIHAHNEAQNETLKGLITNSIENTNNITKNKADIQALENNVVEELFNLSG
RLIDQKADIDNNINNIYELAQQQDQHSSDIKTLKKNVEEGLLELSGHLIDQKTDIAQNQANIQDLATYNE
LQDQYAQKQTEAIDALNKASSENTQNIEDLAAYNELQDAYAKQQTEAIDALNKASSENTQNIEDLAAY
NELQDAYAKQQTEAIDALNKASSENTQNIAKNQADIANNINNIYELAQQQDQHSSDIKTLAKASAANTD
RIAKNKADADASFETLTKNQNTLIEKDKEHDKLITANKTAIDANKASADTKFAATADAITKNGNAITKNAK
SITDLGTKVDGFDSRVTALDTKVNAFDGRITALDSKVENGMAAQAAHH
SEQ ID NO: 20 MC-010 (Protein) - (M)(UspA2 amino acids 30-564)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAI
AKNNESIEDLYDFGHEVAESIGEIHAHNEAQNETLKGLITNSIENTNNITKNKADIQALENNVVEELFNLS
GRLIDQKADIDNNINNIYELAQQQDQHSSDIKTLKKNVEEGLLELSGHLIDQKTDIAQNQANIQDLATYN
ELQDQYAQKQTEAIDALNKASSENTQNIEDLAAYNELQDAYAKQQTEAIDALNKASSENTQNIEDLAA
YNELQDAYAKQQTEAIDALNKASSENTQNIAKNQADIANNINNIYELAQQQDQHSSDIKTLAKASAANT
DRIAKNKADADASFETLTKNQNTLIEKDKEHDKLITANKTAIDANKASADTKFAATADAITKNGNAITKNA
KSITDLGTKVDGFDSRVTALDTKVNAFDGRITALDSKVENGMAAQAA
SEQ ID NO: 21 MC-011 (Protein) - (M)(UspA2 amino acids 31-540)(ASHHHHHH)
MAKNDITLEDLPYLIKKIDQNELEADIGDITALEKYLALSQYGNILALEELNKALEELDEDVGWNQNDIAN
LEDDVETLTKNQNALAEQGEAIKEDLQGLADFVEGQEGKILQNETSIKKNTQRNLVNGFEIEKNKDAIA
KNNESIEDLYDFGHEVAESIGEIHAHNEAQNETLKGLITNSIENTNNITKNKADIQALENNVVEELFNLSG
RLIDQKADIDNNINNIYELAQQQDQHSSDIKTLKKNVEEGLLELSGHLIDQKTDIAQNQANIQDLATYNE
LQDQYAQKQTEAIDALNKASSENTQNIEDLAAYNELQDAYAKQQTEAIDALNKASSENTQNIEDLAAY
NELQDAYAKQQTEAIDALNKASSENTQNIAKNQADIANNINNIYELAQQQDQHSSDIKTLAKASAANTD
RIAKNKADADASFETLTKNQNTLIEKDKEHDKLITANKTAIDANKASADTKFAATADAITKNGNAITKNAK
SITDLGTKVDGFDSRVTALDTKASHHHHHH
SEQ ID NO: 22 UspA2 American 2933 (613 aa)
MKTMKLLPLKIAVTSAMIIGLGAASTANAQSRDRSLEDIQDSISKLVQDDINTLKQDQQKMNKYLLLNQL
ANTLITDELNNNVIKNTNSIEALGDEIGWLENDIADLEEGVEELTKNQNTLIEKDEEHDRLIAQNQADIQT
LENNVVEELFNLSGRLIDQEADIAKNNASIEELYDFDNEVAERIGEIHAYTEEVNKTLENLITNSVKNTDN
IDKNKADIDNNINHIYELAQQQDQHSSDIKTLKNNVEEGLLELSGHLIDQKADLTKDIKALESNVEEGLL
DLSGRLLDQKADLTKDIKALESNVEEGLLDLSGRLLDQKADIAQNQTDIQDLAAYNELQDQYAQKQTE
AIDALNKASSENTQNIEDLAAYNELQDAYAKQQTEAIDALNKASSENTQNIAKNQADIANNINNIYELAQ
QQDQHSSDIKTLAKASAANTNRIATAELGIAENKKDAQIAKAQANANKTAIDENKASADTKFAATADAIT
KNGNAITKNAKSITDLGTKVDGFDGRVTALDTKVNAFDGRITALDSKVENGMAAQAALSGLFQPYSVG
KFNATAALGGYGSKSAVAIGAGYRVNPNLAFKAGAAINTSGNKKGSYNIGVNYEF
SEQ ID NO: 23 UspA2 American 2912 (644 aa)
MKTMKLLPLKIAVTSALIIGLGAASTANAQQQLQTETFLPNFLSNDNYDLTDPFYHNMILGDTALLDKQD
GSQPQLKFYSNDKDSVPDSLLFSKLLHEQQLNGFKKGDTIIPLDKDGKPVYQVDYKLDGKGKKQKRR
QVYSVTTKTATDDDVNSAYSRGILGKVDDLDDEMNFLNHDITSLYDVTANQQDAIKDLKKGVKGLNKE
LKELDKEVGVLSRDIGSLNDDVAQNNESIEDLYDFSQEVADSIGEIHAHNKAQNETLQDLITNSVENTN
NITKNKADIQALENNVVEELFNLSGRLIDQKADLTKDIKTLESNVEEGLLELSGHLIDQKADIAKNQADIA
QNQANIQDLAAYNELQDAYAKQQTEAIDALNKASSENTQNIEDLAAYNELQDAYAKQQTEAIDALNKA
SSENTQNIAKNQADIANNINNIYELAQQQDQHSSDIKTLAKASAANTDRIAKNKADADASFETLTKNQN
TLIEKDKEHDKLITANKTAIDENKASADTKFAATADAITKNGNAITKNAKSITDLGTKVDGFDSRVTALDT
KVNAFDGRITALDSKVENGMAAQAALSGLFQPYSVGKFNATAALGGYGSKSAVAIGAGYRVNPNLAF
KAGAAINTSGNKKGSYNIGVNYEF
SEQ ID NO: 24 UspA2 American 2908 (591 aa)
MKTMKLLPLKIAVTSALIVGLGAASTANAQLVERFFPNIFLDKPLAKQHYHNVVVGDTSIVSDLQSNSD
QLKFYSDDEGLVPDSLLFNKMLHEQLLNGFKEGDTIIPLDENGKPVYKVDYKLDGKEPRKVYSVTTKIA
TAEDVATSSYANGIQKDIDDLYDFDHQVTERLTQHGKTIYRNGERILANEESVQYLNKEVQNNIEHIYE
LAQQQDQHSSDIKTLESNVEKGLLELSGHLIDQKADLTKDIKTLESNVEEGLLDLSGRLIDQKADLTKDI
KTLESNVEEGLLDLSGRLIDQKADIAQNQANIQDLAAYNELQDQYAQKQTEAIDALNKASSENTQNIED
LAAYNELQDAYAKQQTEAIDALNKASSENTQNIAKNQADIANNINNIYELAQQQDQHSSDIKTLAKASA
ANTNRIATAELGIAENKKDAQIAKAQANANKTAIDENKASADTKFAATADAITKNGNAITKNAKSITDLGT
KVDGFDSRVTALDTKVNAFDGRITALDSKVENGMAAQAALSGLFQPYSVGKFNATAALGGYGSKSAV
AIGAGYRVNPNLAFKAGAAINTSGNKKGSYNIGVNYEF
SEQ ID NO: 25 UspA2 Finnish 307 (687 aa)
MKTMKLLPLKIAVTSAMIIGLGAASTANAQQQQQQQQQQQSRTEIFFPNIFFNENHDELDDAYHNIILG
DTALLDKQDGSQPQLKFYSNDKDSVPDSLLFSKLLHEQQLNGFKKGDTIIPLDKDGKPVYQVDYKLDG
KGKKQKRRQVYSVTTKTATDDDVNSAYSRGILGKVDDLDDEMNFLNHDITSLYDVTANQQDAIKGLKK
GVKGLNKELKELDKEVGVLSRDIGSLNDDVAQNNESIEDLYDFSQEVADSIGEIHAHNKAQNETLQDLI
TNSVENTNNITKNKADIQALENNVVEELFNLSGRLIDQKADLTKDIKTLESNVEEGLLELSGHLIDQKADI
AKNQADIAQNQANIQDLAAYNELQDAYAKQQTEAIDALNKASSENTQNIEDLAAYNELQDAYAKQQTE
AIDALNKASSENTQNIEDLAAYNELQDAYAKQQTEAIDALNKASSENTQNIAKNQADIANNINNIYELAQ
QQDQHSSDIKTLAKASAANTDRIAKNKADADASFETLTKNQNTLIEKDKEHDKLITANKTAIDENKASAD
TKFAATADAITKNGNAITKNAKSITDLGTKVDAFDGRVTALDTKVNAFDGRITALDSKVENGMAAQAAL
SGLFQPYSVGKFNATAALGGYGSKSAVAIGAGYRVNPNLAFKAGAAINTSGNKKGSYNIGVNYEF
SEQ ID NO: 26 UspA2 Finnish 353 (683 amino acids)
MKTMKLLPLKIAVTSAMIVGLGMASTANAQQQKSPKTETFLPNIFFNEYADDLDTLYHNMILGDTAITH
DDQYKFYADDATEVPDSLFFNKILHDQLLYGFKEGDKIIPLDENGKPVYKLDKRLENGVQKTVYSVTTK
TATADDVNSAYSRGIQGDIDDLYEANKENVNRLIEHGDKIFANEESVQYLNREVQNNIENIHELAQQQD
QHSSDIKTLKKNVEKDLLDLSGRLIAQKEDIAQNQTDIQDLATYNELQDQYAQKQTEAIDALNKASSEN
TQNIAKNSNHIKTLENNIEEGLLELSGHLIDQKADLTKDIKALESNVEEGLLDLSGRLIDQKADIAQNQAN
IQDLAAYNELQDAYAKQQTEAIDALNKASSENTQNIEDLAAYNELQDAYAKQQTEAIDALNKASSENT
QNIEDLAAYNELQDAYAKQQTEAIDALNKASSENTQNIAKNQADIANNINNIYELAQQQDQHSSDIKTLA
KASAANTDRIAKNKADADASFETLTKNQNTLIEKDKEHDKLITANKTAIDANKASADTKFAATADAITKN
GNAITKNAKSITDLGTKVDGFDGRVTALDTKVNALDTKVNAFDGRITALDSKVENGMAAQAALSGLFQ
PYSVGKFNATAALGGYGSKSAVAIGAGYRVNPNLAFKAGAAINTSGNKKGSYNIGVNYEF
SEQ ID NO: 27 UspA2 Finnish 358 (684 amino acids)
MKTMKLLPLKIAVTSAMMVGLGMASTANAQQQKSPKTEIFLPNLFDNDNTELTDPLYHNMILGNTALLT
QENQYKFYADDGNGVPDSLLFNKILHDQLLHGFKEGGTIIPLDENGKPVYKLDSIVEQGKTKTVYSVTT
KTATADDVNSAYSRGIQGDIDDLYEANKENVNRLIEHGDKIFANEESVQYLNREVQNNIENIHELAQQQ
DQHSSDIKTLKKNVEKDLLDLSGRLIAQKEDIAQNQTDIQDLATYNELQDQYAQKQTEAIDALNKASSE
NTQNIAKNSNHIKTLENNIEEGLLELSGHLIDQKADLTKDIKALESNVEEGLLDLSGRLIDQKADIAQNQA
NIQDLAAYNELQDAYAKQQTEAIDALNKASSENTQNIEDLAAYNELQDAYAKQQTEAIDALNKASSENT
QNIEDLAAYNELQDAYAKQQTEAIDALNKASSENTQNIAKNQADIANNINNIYELAQQQDQHSSDIKTLA
KASAANTDRIAKNKADADASFETLTKNQNTLIEKDKEHDKLITANKTAIDANKASADTKFAATADAITKN
GNAITKNAKSITDLGTKVDGFDGRVTALDTKVNALDTKVNAFDGRITALDSKVENGMAAQAALSGLFQ
PYSVGKFNATAALGGYGSKSAVAIGAGYRVNPNLAFKAGAAINTSGNKKGSYNIGVNYEF
SEQ ID NO: 28 UspA2 Finnish 216 (684 amino acids)
MKTMKLLPLKIAVTSAMIIGLGAASTANAQQQQKTKTEVFLPNLFDNDYYDLTDPLYHSMILGDTATLF
DQQDNSKSQLKFYSNDKDSVPDSLLFSKLLHEQQLNGFKAGDTIIPLDKDGKPVYTQDTRTKDGKVET
VYSVTTKIATQDDVEQSAYSRGIQGDIDDLYDINREVNEYLKATHDYNERQTEAIDALNKASSANTDRI
DTAEERIDKNEYDIKALESNVGKDLLDLSGRLIAQKEDIDNNINHIYELAQQQDQHSSDIKTLKNNVEEG
LLELSGHLIDQKADLTKDIKTLENNIEEGLLELSGHLIDQKADLTKDIKTLENNIEEGLLELSGHLIDQKAD
IAQNQANIQDLAAYNELQDQYAQKQTEAIDALNKASSENTQNIEDLAAYNELQDAYAKQQTEAIDALNK
ASSENTQNIEDLAAYNELQDAYAKQQTEAIDALNKASSENTQNIAKNQADIANNINNIYELAQQQDQHS
SDIKTLAKVSAANTDRIAKNKADADASFETLTKNQNTLIEKDKEHDKLITANKTAIDANKASADTKFAAT
ADAITKNGNAITKNAKSITDLGTKVDGFDGRVTALDTKVNAFDGRITALDSKVENGMAAQAALSGLFQP
YSVGKFNATAALGGYGSKSAVAIGAGYRVNPNLAFKAGAAINTSGNKKGSYNIGVNYEF
SEQ ID NO: 29 UspA2 Dutch H2 (684 amino acids)
MKTMKLLPLKIAVTSAMMVGLGMASTANAQQQKSPKTEIFLPNLFDNDNTELTDPLYHNMILGNTALLT
QENQYKFYADDGNGVPDSLLFNKILHDQLLHGFKKGDTIIPLDENGKPVYKLDSIVEQGKTKTVYSVTT
KTATADDVNSAYSRGIQGDIDDLYEANKENVNRLIEHGDKIFANEESVQYLNREVQNNIENIYELVQQQ
DQHSSDIKTLKKNVEKDLLDLSGRLIAQKEDIAQNQTDIQDLATYNELQDQYAQKQTEAIDALNKASSE
NTQNIAKNSNHIKTLENNIEEGLLELSGHLIDQKADLTKDIKALESNVEEGLLDLSGRLIDQKADIAQNQA
NIQDLAAYNELQDAYAKQQTEAIDALNKASSENTQNIEDLAAYNELQDAYAKQQTEAIDALNKASSENT
QNIEDLAAYNELQDAYAKQQTEAIDALNKASSENTQNIAKNQADIANNINNIYELAQQQDQHSSDIKTLA
KASAANTDRIAKNKADADASFETLTKNQNTLIEKDKEHDKLITANKTAIDANKASADTKFAATADAITKN
GNAITKNAKSITDLGTKVDGFDGRVTALDTKVNALDTKVNAFDGRITALDSKVENGMAAQAALSGLFQ
PYSVGKFNATAALGGYGSKSAVAIGAGYRVNPNLAFKAGAAINTSGNKKGSYNIGVNYEF
SEQ ID NO: 30 UspA2 Dutch F10 (574 amino acids)
MKTMKLLPLKIAVTSAMIIGLGAASTANAQLAEQFFPNIFSNHAPVKQHYHNVVVGDTSIVENLQDSDD
TQLKFYSNDEYSVPDSLLFNKMLHEQQLNGFKKGDTIIPLDENGKPVYKVDYKLDGQEPRRVYSVTTK
IATQDDVDNSPYSRGIQGDIDDLYEANKENVNRLIEHGDKIFANEESVQYLNKEVQNNIENIYELAQQQ
DQHSSDIKTLKKNVEEGLLELSGHLIDQKADLTKDIKTLESNVEEGLLELSGHLIDQKADIAKNQADIAQ
NQANIQDLAAYNELQDAYAKQQTEAIDALNKASSENTQNIEDLAAYNELQDAYAKQQTEAIDALNKAS
SENTQNIAKNQADIANNINNIYELAQQQDQHSSDIKTLAKASAANTDRIAKNKADADASFETLTKNQNT
LIEKDKEHDKLITANKTAIDANKASADTKFAATADAITKNGNAITKNAKSITDLGTKVDAFDGRVTALDTK
VNAFDGRITALDSKVENGMAAQAALSGLFQPYSVGKFNATAALGGYGSKSAVAIGAGYRVNPNLAFK
AGAAINTSGNKKGSYNIGVNYEF
SEQ ID NO: 31 UspA2 Norwegian 1(678 amino acids)
MKTMKLLPLKIAVTSALIVGLGAASTANAQQQPQTETFFPNIFFNENHDALDDVYHNMILGDTAITQDN
QYKFYADAISEVPDSLLFNKILHDQQLNGFKEGDTIIPLDENGKPVYKLDEKVENGVKKSVYSVTTKTA
TRADVEQSAYSRGIQGDIDDLYEANKENVNRLIEHGDKIFANEESVQYLNKEVQNNIENIHELAQQQD
QHSSDIKTLKKNVEEGLLELSGHLIDQKADLTKDIKTLESNVEEGLLDLSGRLLDQKADIAQNQANIQDL
AAYNELQDAYAKQQTEAIDALNKASSENTQNIEDLAAYNELQDAYAKQQTEAIDALNKASSENTQNIE
DLAAYNELQDAYAKQQTEAIDALNKASSENTQNIEDLAAYNELQDAYAKQQTEAIDALNKASSENTQNI
EDLAAYNELQDAYAKQQTEAIDALNKASSENTQNIAKNQADIANNINNIYELAQQQDQHSSDIKTLAKA
SAANTDRIAKNKADADASFETLTKNQNTLIEKDKEHDKLITANKTAIDANKASADTKFAATADAITKNGN
AITKNAKSITDLGTKVDAFDGRVTALDTKVNALDTKVNAFDGRITALDSKVENGMAAQAALSGLFQPYS
VGKFNATAALGGYGSKSAVAIGAGYRVNPNLAFKAGAAINTSGNKKGSYNIGVNYEF
SEQ ID NO: 32 UspA2 Norwegian 13 (678 amino acids)
MKTMKLLPLKIAVTSAMIVGLGAASTANAQQQQQPRTETFFPNIFFNENHDALDDVYHNMILGDTAITQ
DNQYKFYADAISEVPDSLLFNKILHDQQLNGFKEGDTIIPLDENGKPVYKLDEKVENGVKKSVYSVTTK
TATRADVEQSAYSRGIQGDIDDLYEANKENVNRLIEHGDKIFANEESVQYLNREVQNNIENIHELAQQQ
DQHSSDIKTLKKNVEKDLLDLSGRLIAQKEDIAQNQTDIQDLATYNELQDQYAQKQTEAIDALNKASSE
NTQNIAKNSNHIKTLENNIEEGLLELSGHLIDQKADLTKDIKTLENNIEEGLLELSGHLIDQKADLTKDIKA
LESNVEEGLLDLSGRLLDQKADIAQNQANIQDLAAYNELQDQYAQKQTEAIDALNKASSENTQNIEDL
AAYNELQDAYAKQQTEAIDALNKASSENTQNIAKNQADIANNINNIYELAQQQDQHSSDIKTLAKASAA
NTDRIAKNKADADASFETLTKNQNTLIEKDKEHDKLITANKTAIDTNKASADTKFAATADAITKNGNAITK
NAKSITDLGTKVDGFDGRVTALDTKVNALDTKVNAFDGRITALDSKVENGMAAQAALSGLFQPYSVGK
FNATAALGGYGSKSAVAIGAGYRVNPNLAFKAGAAINTSGNKKGSYNIGVNYEF
SEQ ID NO: 33 UspA2 Norwegian 33 (587 amino acids)
MKTMKLLPLKIAVTSALIVGLGAASTANAQLVERFFPNIFLDKPLAKQHYHNVVVGDTSIVSDLQSNSD
QLKFYSDDEGLVPDSLLFNKMLHEQLLNGFKEGDTIIPLDENGKPVYKVDYKLDGKEPRKVYSVTTKIA
TAEDVATSSYANGIQKDIDDLYDFDHQVTERLTQHGKTIYRNGERILANEESVQYLNKEVQNNIEHIYE
LAQQQDQHSSDIKTLESNVEKGLLELSGHLIDQKADLTKDIKTLENNVEEGLLDLSGRLIDQKADIAQN
QANIQDLAAYNELQDQYAQKQTEAIDALNKASSENTQNIEDLAAYNELQDAYAKQQTEAIDALNKASS
ENTQNIAKNQADIANNINNIYELAQQQDQHSSDIKTLAKASAANTDRIAKNKADADASFETLTKNQNTLI
EKDKEHDKLITANKTAIDTNKASADTKFAATADAITKNGNAITKNAKSITDLGTKVDGFDSRVTALDTKV
NALDTKVNALDTKVNAFDGRITALDSKVENGMAAQAALSGLFQPYSVGKFNATAALGGYGSKSAVAI
GAGYRVNPNLAFKAGAAINTSGNKKGSYNIGVNYEF
SEQ ID NO: 34 UspA2 Norwegian 25 (678 amino acids)
MKTMKLLPLKIAVTSAMIVGLGAASTANAQQQQQPRTETFFPNIFFNENHDALDDVYHNMILGDTAITQ
DNQYKFYADAISEVPDSLLFNKILHDQQLNGFKEGDTIIPLDENGKPVYKLDEKVENGVKKSVYSVTTK
TATRADVEQSAYSRGIQGDIDDLYEANKENVNRLIEHGDKIFANEESVQYLNREVQNNIENIHELAQQQ
DQHSSDIKTLKKNVEKDLLDLSGRLIAQKEDIAQNQTDIQDLATYNELQDQYAQKQTEAIDALNKASSE
NTQNIAKNSNHIKTLENNIEEGLLELSGHLIDQKADLTKDIKTLENNIEEGLLELSGHLIDQKADLTKDIKA
LESNVEEGLLDLSGRLLDQKADIAQNQANIQDLAAYNELQDQYAQKQTEAIDALNKASSENTQNIEDL
AAYNELQDAYAKQQTEAIDALNKASSENTQNIAKNQADIANNINNIYELAQQQDQHSSDIKTLAKASAA
NTDRIAKNKADADASFETLTKNQNTLIEKDKEHDKLITANKTAIDTNKASADTKFAATADAITKNGNAITK
NAKSITDLGTKVDGFDGRVTALDTKVNALDTKVNAFDGRITALDSKVENGMAAQAALSGLFQPYSVGK
FNATAALGGYGSKSAVAIGAGYRVNPNLAFKAGAAINTSGNKKGSYNIGVNYEF
SEQ ID NO: 35 UspA2 Norwegian 27 (616 amino acids)
MKTMKLLPLKIAVTSALIVGLGAASTANAQVRDKSLEDIEALLGKIDISKLEKEKKQQTELQKYLLLSQYA
NVLTMEELNKNVEKNTNSIEALGYEIGWLENDIADLEEGVEELTKNQNTLIEKDEEHDRLIAQNQADIKT
LENNVVEELFNLSDRLIDQEADIAKNNASIEELYDFDNEVAERIGEIHAYTEEVNKTLEKLITNSVKNTDN
IDKNKADIQALENNVEEGLLELSGHLIDQKADLTKDIKALESNVEEGLLDLSGRLLDQKADIAKNQADIA
QNQTDIQDLAAYNELQDQYAQKQTEAIDALNKASSENTQNIEDLAAYNELQDAYAKQQTEAIDALNKA
SSENTQNIEDLAAYNELQDAYAKQQTEAIDALNKASSENTQNIAKNQADIANNINNIYELAQQQDQHSS
DIKTLAKVSAANTDRIAKNKADADASFETLTKNQNTLIEKDKEHDKLITANKTAIDANKASADTKFAATA
DAITKNGNAITKNAKSITDLGTKVDGFDSRVTALDTKVNAFDGRITALDSKVENGMAAQAALSGLFQPY
SVGKFNATAALGGYGSKSAVAIGAGYRVNPNLAFKAGAAINTSGNKKGSYNIGVNYEF
SEQ ID NO: 36 UspA2 Norwegian 36 (676 amino acids)
MKTMKLLPLKIAVTSALIVGLGAASTANAQATETFLPNLFDNDYTETTDPLYHGMILGNTAITQDTQYKF
YAENGNEVPDSLFFNKILHDQQLNGFKEGDTIIPLDENGKPVYKLDEITENGVKRKVYSVTTKTATRED
VEQSAYSRGIQGDIDDLYEANKENVNRLIEHGDKIFANEESVQYLNKEVQNNIENIHELAQQQDQHSS
DIKTLKKNVEEGLLELSGHLIDQKADLTKDIKALESNVEEGLLDLSGHLIDQKADLTKDIKALESNVEEGL
LDLSGRLLDQKADIAKNQADIAQNQTDIQDLAAYNELQDQYAQKQTEAIDALNKASSENTQNIEDLAAY
NELQDQYAQKQTEAIDALNKASSENTQNIEDLAAYNELQDQYAQKQTEAIDALNKASSENTQNIEDLA
AYNELQDQYAQKQTEAIDALNKASSENTQNIAKNQADIANNINNIYELAQQQDQHSSDIKTLAKASAAN
TDRIAKNKADADASFETLTKNQNTLIEKDKEHDKLITANKTAIDANKASADTKFAATADAITKNGNAITKN
AKSITDLGTKVDGFDGRVTALDTKVNALDTKVNAFDGRITALDSKVENGMAAQAALSGLFQPYSVGKF
NATAALGGYGSKSAVAIGAGYRVNPNLAFKAGAAINTSGNKKGSYNIGVNYEF
SEQ ID NO: 37 UspA2 BC5SV (629 amino acids)
MKTMKLLPLKIAVTSALIVGLGAASTANAQNGTSTKLKNLKEYAQYLDNYAQYLDDDIDDLDKEVGELS
QNIAKNQANIKDLNKKLSRDIDSLREDVYDNQYEIVNNQADIEKNQDDIKELENNVGKELLNLSGRLLD
QKADIDNNINNIYELAQQQDQHSSDIKTLKKNVEEGLLELSGHLIDQKSDIAQNQTDIQDLATYN ELQD
QYAQKQTEAIDALNKASSENTQNIEDLAAYNELQDAYAKQQTEAIDALNKASSENTQNIQDLAAYNEL
QDAYAKQQTEAIDALNKASSENTQNIEDLAAYNELQDAYAKQQTEAIDALNKASSENTQNIEDLAAYN
ELQDAYAKQQTEAIDALNKASSENTQNIEDLAAYNELQDAYAKQQTEAIDALNKASSENTQNIAKNQA
DIANNINNIYELAQQQDQHSSDIKTLAKASAANTDRIAKNKADADASFETLTKNQNTLIEKDKEHDKLITA
NKTAIDANKASADTKFAATADAITKNGNAITKNAKSITDLGTKVDAFDGRVTALDTKVNAFDGRITALDS
KVENGMAAQAALSGLFQPYSVGKFNATAALGGYGSKSAVAIGAGYRVNPNLAFKAGAAINTSGNKKG
SYNIGVNYEF
SEQ ID NO: 38 UspA2 Norwegian 14 (683 amino acids)
MKTMKLLPLKIAVTSAMIVGLGMASTANAQQQRSPKTETFLPNIFFNEYADDLDTLYHNMILGDTAITH
DDQYKFYADDATEVPDSLFFNKILHDQLLYGFKEGDKIIPLDENGKPVYKLDKRLDNGVQKTVYSVTTK
TATADDVNSAYSRGIQGDIDDLYEANKENVNRLIEHGDKIFANEESVQYLNKEVQNNIENIHELAQQQD
QHSSDIKTLKKNVEEGLLELSGHLIDQKTDIAQNQTDIQDLATYNELQDQYAQKQTEAIDALNKASSEN
TQNIAKNSNRIKALENNIEEGLLELSGHLIDQKADLTKDIKALESNVEEGLLDLSGRLIDQKADIAQNQAN
IQDLAAYNELQDAYAKQQTEAIDALNKASSENTQNIEDLAAYNELQDAYAKQQTEAIDALNKASSENT
QNIEDLAAYNELQDAYAKQQTEAIDALNKASSENTQNIAKNQADIANNINNIYELAQQQDQHSSDIKTLA
KASAANTDRIAKNKADADASFETLTKNQNTLIEKDKEHDKLITANKTAIDANKASADTKFAATADAITKN
GNAITKNAKSITDLGTKVDGFDGRVTALDTKVNALDTKVNAFDGRITALDSKVENGMAAQAALSGLFQ
PYSVGKFNATAALGGYGSKSAVAIGAGYRVNPNLAFKAGAAINTSGNKKGSYNIGVNYEF
SEQ ID NO: 39 UspA2 Norwegian 3 (700 amino acids)
MKTMKLLPLKIAVTSAMIVGLGAASTANAQAQSNRSLDQVQALLRGIDETKIKKEIQQSQQPELNKYLT
FNQLANALNIEELNNNVQKNTQRLDSAATLYGDLSKTVPKSIKENKESIKENKESIKENKESIKENKESI
KENKESIKENKESITTLTRKSFQNQVDIVRNNASIEDLYAYGQEVAKSIGEIHAYTEEVNKTLENLITNSV
ENTNNITKNKADIQALENNVVEELFNLSGRLIDQKADIDNNINNIYELAQQQDQHSSDIKTLKKNVEEGL
LELSGHLIDQKADLTKDIKTLESNVEEGLLDLSGRLLDQKADIAQNQANIQDLAAYNELQDAYAKQQTE
AIDALNKASSENTQNIEDLAAYNELQDAYAKQQTEAIDALNKASSENTQNIEDLAAYNELQDAYAKQQT
EAIDALNKASSENTQNIEDLAAYNELQDAYAKQQTEAIDALNKASSENTQNIAKNQADIANNINNIYELA
QQQDQHSSDIKTLAKASAANTDRIAKNKADADASFETLTKNQNTLIEKDKEHDKLITANKTVIDANKAS
ADTKFAATADAITKNGNAITKNAKSITDLGTKVDGFDGRVTALDTKVNALDTKVNAFDGRITALDSKVE
NGMAAQAALSGLFQPYSVGKFNATAALGGYGSKSAVAIGAGYRVNPNLAFKAGAAINTSGNKKGSYN
IGVNYEF
SEQ ID NO: 40 UspA2 Finnish 414 (676 amino acids)
MKTMKLLPLKIAVTSALIVGLGAASTANAQATETFLPNLFDNDYIETTDPLYHGMILGNTAITQDTQYKF
YAENGNEVPDSLFFNKILHDQQLNGFKEGDTIIPLDENGKPVYKLDEITENGVKRKVYSVTTKTATRED
VEQSAYSRGIQGDIDDLYEANKENVNRLIEHGDKIFANEESVQYLNKEVQNNIENIHELAQQQDQHSS
DIKTLKKNVEEGLLELSGHLIDQKADLTKDIKTLENNVEEGLLELSGHLIDQKADLTKDIKALESNVEEGL
LDLSGRLLDQKADIAKNQADIAQNQTDIQDLAAYNELQDQYAQKQTEAIDALNKASSENTQNIEDLAAY
NELQDQYAQKQTEAIDALNKASSENTQNIEDLAAYNELQDQYAQKQTEAIDALNKASSENTQNIEDLA
AYNELQDQYAQKQTEAIDALNKASSENTQNIAKNQADIANNINNIYELAQQQDQHSSDIKTLAKASAAN
TDRIAKNKADADASFETLTKNQNTLIEKDKEHDKLITANKTAIDANKASADTKFAATADAITKNGNAITKN
AKSITDLGTKVDGFDGRVTALDTKVNALDTKVNAFDGRITALDSKVENGMAAQAALSGLFQPYSVGKF
NATAALGGYGSKSAVAIGAGYRVNPNLAFKAGAAINTSGNKKGSYNIGVNYEF
SEQ ID NO: 41 UspA2 Japanese Z7476 (678 amino acids)
MKTMKLLPLKIAVTSAMIIGLGAASTANAQLAEQFFPNIFSNHAPVKQHYHNVVVGDTSIVENLQDSDD
TQLKFYSNDEYSVPDSLLFNKMLHEQQLNGFKKGDTIIPLDENGKPVYKVDYKLDGQEPRRVYSVTTK
IATQDDVDNSPYSRGIQGDIDDLYEANKENVNRLIEHGDKIFANEESVQYLNKEVQNNIENIYELAQQQ
DQHSSDIKTLKKNVEEGLLELSGRLIDQKADIAQNQANIQDLAAYNELQDQYAQKQTEAIDALNKASSE
NTQNIEDLAAYNELQDAYAKQQTEAIDALNKASSENTQNIEDLAAYNELQDAYAKQQTEAIDALNKASS
ENTQNIEDLAAYNELQDAYAKQQTEAIDALNKASSENTQNIEDLAAYNELQDAYAKQQTEAIDALNKAS
SENTQNIEDLAAYNELQDAYAKQQTEAIDALNKASSENTQNIAKNQADIANNINNIYELAQQQDQHSSD
IKTLAKVSAANTDRIAKNKADADASFETLTKNQNTLIEKDKEHDKLITANKTAIDANKASADTKFAATAD
AITKNGNAITKNAKSITDLGTKVDGFDGRVTALDTKVNAFDGRITALDSKVENGMAAQAALSGLFQPYS
VGKFNATAALGGYGSKSAVAIGAGYRVNPNLAFKAGAAINTSGNKKGSYNIGVNYEF
SEQ ID NO: 42 UspA2 Belgian Z7530 (613 amino acids)
MKTMKLLPLKIAVTSAMIIGLGAASTANAQSRDRSLEDIQDSISKLVQDDINTLKQDQQKMNKYLLLNQL
ANTLITDELNNNVIKNTNSIEALGDEIGWLENDIADLEEGVEELTKNQNTLIEKDEEHDRLIAQNQADIQT
LENNVVEELFNLSGRLIDQEADIAKNNASIEELYDFDNEVAERIGEIHAYTEEVNKTLENLITNSVKNTDN
IDKNKADIDNNINHIYELAQQQDQHSSDIKTLKNNVEEGLLELSGHLIDQKADLTKDIKALESNVEEGLL
DLSGRLLDQKADLTKDIKALESNVEEGLLDLSGRLLDQKADIAQNQTDIQDLAAYNELQDQYAQKQTE
AIDALNKASSENTQNIEDLAAYNELQDAYAKQQTEAIDALNKASSENTQNIAKNQADIANNINNIYELAQ
QQDQHSSDIKTLAKASAANTNRIATAELGIAEN KKDAQIAKAQANANKTAIDENKASADTKFAATADAIT
KNGNAITKNAKSITDLGTKVDGFDGRVTALDTKVNAFDGRITALDSKVENGMAAQAALSGLFQPYSVG
KFNATAALGGYGSKSAVAIGAGYRVNPNLAFKAGAAINTSGNKKGSYNIGVNYEF
SEQ ID NO: 43 German Z8063 (589 amino acids)
MKTMKLLPLKIAVTSALIVGLGAASTANAQATNKDITLEDVLKSIEEIDPYELRDYIEYPTAIERFLLLSQY
GNTLTLEEFDNDIELLDQDVEDLEESVTELAKNQNSLIEQGEAIKEDLQGLADFVERQEDKILQNETSIK
KNTQRNLVNGFEIEKNKDAIAKNNESIEDLYDFGHEVAKSIGEIHAHNEAQNETLKDLITNSVKNTDNIT
KNKADIQALESNVEKGLLELSGHLIDQKADIDNNINNIHELAQQQDQHSSDIKTLKKNVEEGLLELSGHL
IDQKSDIAQNQANIQDLATYNELQDQYAQKQTEAIDALNKASSENTQNIEDLAAYNELQDAYAKQQTE
AIDALNKASSENTQNIAKNQADIANNINNIYELAQQQDQHSSDIKTLAKASAANTDRIAKNKADADASFE
TLTKNQNTLIEKDKEHDKLITANKTAIDANKASADTKFAATADAITKNGNAITKNAKSITDLGTKVDGFDS
RVTALDTKVNAFDGRITALDSKVENGMAAQAALSGLFQPYSVGKFNATAALGGYGSKSAVAIGAGYR
VNPNLAFKAGAAINTSGNKKGSYNIGVNYEF
SEQ ID NO: 44 UspA2 American 012E (684 amino acids)
MKTMKLLPLKIAVTSAMMVGLGMASTANAQQQKSPKTEIFLPNLFDNDNTELTDPLYHNMILGNTALLT
QENQYKFYADDGNGVPDSLLFNKILHDQLLHGFKEGDTIIPLDENGKPVYKLDSIVEQGKTKTVYSVTT
KTATADDVNSAYSRGIQGDIDDLYEANKENVNRLIEHGDKIFANEESVQYLNREVQNNIENIHELAQQQ
DQHSSDIKTLKKNVEKDLLDLSGRLIAQKEDIAQNQTDIQDLATYNELQDQYAQKQTEAIDALNKASSE
NTQNIAKNSNHIKTLENNIEEGLLELSGHLIDQKADLTKDIKALESNVEEGLLDLSGRLIDQKADIAQNQA
NIQDLAAYNELQDAYAKQQTEAIDALNKASSENTQNIEDLAAYNELQDAYAKQQTEAIDALNKASSENT
QNIEDLAAYNELQDAYAKQQTEAIDALNKASSENTQNIAKNQADIANNINNIYELAQQQDQHSSDIKTLA
KASAANTDRIAKNKADADASFETLTKNQNTLIEKDKEHDKLITANKTAIDANKASADTKFAATADAITKN
GNAITKNAKSITDLGTKVDGFDGRVTALDTKVNALDTKVNAFDGRITALDSKVENGMAAQAALSGLFQ
PYSVGKFNATAALGGYGSKSAVAIGAGYRVNPNLAFKAGAAINTSGNKKGSYNIGVNYEF
SEQ ID NO: 45 UspA2 Greek MC317 (650 amino acids)
MKTMKLLPLKIAVTSALIVGLGAASTANAQQQQKTKTEVFLPNLFYNDYIEETDLLYHNMILGDTAALVD
RQNYSNSQLKFYSNDEESVPDSLLFSKMLNNQQLNGFKAGDIIIPVDANGQVIYQKDTRVEGGKTRTV
LSVTTKIATQQDVDSAYSRGIQGKVNDLDDEMNFLNHDITSLYDVTANQQDDIKGLKKGVKDLKKGVK
GLNKELKELDKEVGVLSRDIGSLNDDVAQNNESIEDLYDFSQEVADSIGEIHAHNKAQNETLQDLITNS
VENTNNITKNKADIQALENNVVEELFNLSGRLIDQKADLTKDIKTLESNVEEGLLELSGHLIDQKADIAKN
QADIAQNQANIQDLAAYNELQDAYAKQQTEAIDALNKASSENTQNIEDLAAYNELQDAYAKQQTEAID
ALNKASSENTQNIAKNQADIANNINNIYELAQQQDQHSSDIKTLAKASAANTDRIAKNKADADASFETLT
KNQNTLIEKDKEHDKLITANKTAIDENKASADTKFAATADAITKNGNAITKNAKSITDLGTKVDGFDGRV
TALDTKVNAFDGRITALDSKVENGMAAQAALSGLFQPYSVGKFNATAALGGYGSKSAVAIGAGYRVN
PNLAFKAGAAINTSGNKKGSYNIGVNYEF
SEQ ID NO: 46 UspA2 American V1122 (616 amino acids)
MKTMKLLPLKIAVTSALIVGLGAVSTTNAQAQSRSLDQIQTKLADLAGKIAAGKNGGGQNNQNNQNDI
NKYLFLSQYANILTMEELNNNVVKNSSSIETLETDFGWLENDVADLEDGVEELTKNQNTLIEKDEEHDR
LIAQNQADIQTLENNVVEELFNLSDRLIDQKADIAKNQADIAQNNESIEELYDFDNEVAEKIGEIHAYTEE
VNKTLQDLITNSVKNTDNIDKNKADIDNNINHIYELAQQQDQHSSDIKTLKNNVEEGLLELSGHLIDQKA
DLTKDIKTLENNVEEGLLDLSGRLIDQKADIAKNQADIAQNQTDIQDLAAYNELQDQYAQKQTEAIDALN
KASSENTQNIEDLAAYNELQDAYAKQQTEAIDALNKASSENTQNIAKNQADIANNINNIYELAQQQDQH
SSDIKTLAKASAANTDRIAKNKADADASFETLTKNQNTLIEKDKEHDKLITANKTAIDENKASADTKFAA
TADAITKNGNAITKNAKSITDLGTKVDGFDGRVTALDTKVNAFDGRITALDSKVENGMAAQAALSGLFQ
PYSVGKFNATAALGGYGSKSAVAIGAGYRVNPNLAFKAGAAINTSGNKKGSYNIGVNYEF
SEQ ID NO: 47 UspA2 American P44 (668 amino acids)
MKTMKLLPLKIAVTSALIVGLGTASTANAQVASPANQKIQQKIKKVRKELRQDIKSLRNDIDSNTADIGS
LNDDVADNQDDILDNQADIAKNQDDIEKNQADIKELDKEVGVLSREIGSLNDDIADNYTDIIDNYTDIIDN
QANIAKNQDDIEKNQADIKELDKEVGVLSREIGSLNDDVADNQDDIAKNQADIQTLENNVEEGLLELSG
HLLDQKADIDNNINNIYELAQQQDQHSSDIKTLKKNVEEGLLELSGHLIDQKTDIAQNQANIQDLATYNE
LQDQYAQEQTEAIDALNKASSENTQNIAKNSNRIKALESNVEEGLLELSGHLIDQKADLTKDIKALESNV
EEGLLELSGHLIDQKADIAQNQANIQDLAAYNELQDQYAQKQTEAIDALNKASSENTQNIEDLAAYNEL
QDAYAKQQTEAIDALNKASSENTQNIAKNQADIANNINNIYELAQQQDQHSSDIKTLAKASAANTDRIA
KNKADADASFETLTKNQNTLIEKDKEHDKLITANKTAIDANKVSADTKFAATADAITKNGNAITKNAKSIT
DLGTKVDAFDSRVTALDTKVNAFDGRITALDSKVENGMAAQAALSGLFQPYSVGKFNATAALGGYGS
KSAVAIGAGYRVNPNLAFKAGAAINTSGNKKGSYNIGVNYEF
SEQ ID NO: 48 UspA2 American V1171 (674 amino acids)
MKTMKLLPLKIAVTSAMIVGLGATSTVNAQVVEQFFPNIFFNENHDELDDAYHNMILGDTAIVSNSQDN
STQLKFYSNDEDSVPDSLLFSKLLHEQQLNGFKAGDTIIPLDKDGKPVYTKDTRTKDGKVETVYSVTTK
IATQDDVEQSAYSRGIQGDIDDLYDINREVNEYLKATHDYNERQTEAIDALNKASSANTDRIDTAEERID
KNEYDIKALESNVEEGLLELSGHLIDQKADLTKDIKALESNVEEGLLELSGHLIDQKADLTKDIKALESNV
EEGLLDLSGRLIDQKADIAQNQANIQDLAAYNELQDAYAKQQTEAIDALNKASSENTQNIEDLAAYNEL
QDAYAKQQTEAIDALNKASSENTQNIEDLAAYNELQDAYAKQQTEAIDALNKASSENTQNIEDLAAYN
ELQDAYAKQQTEAIDALNKASSENTQNIAKNQADIANNINNIYELAQQQDQHSSDIKTLAKASAANTDRI
AKNKADADASFETLTKNQNTLIEKDKEHDKLITANKTAIDANKASADTKFAATADAITKNGNAITKNAKSI
TDLGTKVDGFDGRVTALDTKVNALDTKVNAFDGRITALDSKVENGMAAQAALSGLFQPYSVGKFNAT
AALGGYGSKSAVAIGAGYRVNPNLAFKAGAAINTSGNKKGSYNIGVNYEF
SEQ ID NO: 49 UspA2 American TTA24 (613 amino acids)
MKTMKLLPLKIAVTSAMIIGLGAASTANAQSRDRSLEDIQDSISKLVQDDIDTLKQDQQKMNKYLLLNQL
ANTLITDELNNNVIKNTNSIEALGDEIGWLENDIADLEEGVEELTKNQNTLIEKDEEHDRLIAQNQADIQT
LENNVVEELFNLSGRLIDQEADIAKNNASIEELYDFDNEVAERIGEIHAYTEEVNKTLENLITNSVKNTDN
IDKNKADIDNNINHIYELAQQQDQHSSDIKTLKNNVEEGLLELSGHLIDQKADLTKDIKALESNVEEGLL
DLSGRLLDQKADLTKDIKALESNVEEGLLDLSGRLLDQKADIAQNQTDIQDLAAYNELQDQYAQKQTE
AIDALNKASSENTQNIEDLAAYNELQDAYAKQQTEAIDALNKASSENTQNIAKNQADIANNINNIYELAQ
QQDQHSSDIKTLAKASAANTNRIATAELGIAENKKDAQIAKAQANANKTAIDENKASADTKFAATADAIT
KNGNAITKNAKSITDLGTKVDGFDGRVTALDTKVNAFDGRITALDSKVENGMAAQAALSGLFQPYSVG
KFNATAALGGYGSKSAVAIGAGYRVNPNLAFKAGAAINTSGNKKGSYNIGVNYEF
SEQ ID NO: 50 UspA2 American O35E (576 amino acids)
MKTMKLLPLKIAVTSAMIVGLGATSTVNAQVVEQFFPNIFFNENHDELDDAYHNMILGDTAIVSNSQDN
STQLKFYSNDEDSVPDSLLFSKLLHEQQLNGFKAGDTIIPLDKDGKPVYTKDTRTKDGKVETVYSVTTK
IATQDDVEQSAYSRGIQGDIDDLYDINREVNEYLKATHDYNERQTEAIDALNKASSANTDRIDTAEERID
KNEYDIKALESNVEEGLLELSGHLIDQKADLTKDIKALESNVEEGLLELSGHLIDQKADLTKDIKALESNV
EEGLLDLSGRLLDQKADIAKNQADIAQNQTDIQDLAAYNELQDAYAKQQTEAIDALNKASSENTQNIAK
NQADIANNINNIYELAQQQDQHSSDIKTLAKASAANTDRIAKNKADADASFETLTKNQNTLIEKDKEHD
KLITANKTAIDANKASADTKFAATADAITKNGNAITKNAKSITDLGTKVDGFDGRVTALDTKVNALDTKV
NAFDGRITALDSKVENGMAAQAALSGLFQPYSVGKFNATAALGGYGSKSAVAIGAGYRVNPNLAFKA
GAAINTSGNKKGSYNIGVNYEF
SEQ ID NO: 51 UspA2 American SP12-6 (684 amino acids)
MKTMKLLPLKIAVTSAMMVGLGMASTANAQQQKSPKTEIFLPNLFDNDNTELTDPLYHNMILGNTALLT
QENQYKFYADDGNGVPDSLLFNKILHDQLLHGFKEGDTIIPLDENGKPVYKLDSIVEQGKTKTVYSVTT
KTATADDVNSAYSRGIQGDIDDLYEANKENVNRLIEHGDKIFANEESVQYLNREVQNNIENIHELAQQQ
DQHSSDIKTLKKNVEKDLLDLSGRLIAQKEDIAQNQTDIQDLATYNELQDQYAQKQTEAIDALNKASSE
NTQNIAKNSNHIKTLENNIEEGLLELSGHLIDQKADLTKDIKALESNVEEGLLDLSGRLIDQKADIAQNQA
NIQDLAAYNELQDAYAKQQTEAIDALNKASSENTQNIEDLAAYNELQDAYAKQQTEAIDALNKASSENT
QNIEDLAAYNELQDAYAKQQTEAIDALNKASSENTQNIAKNQADIANNINNIYELAQQQDQHSSDIKTLA
KASAANTDRIAKNKADADASFETLTKNQNTLIEKDKEHDKLITANKTAIDANKASADTKFAATADAITKN
GNAITKNAKSITDLGTKVDGFDGRVTALDTKVNALDTKVNAFDGRITALDSKVENGMAAQAALSGLFQ
PYSVGKFNATAALGGYGSKSAVAIGAGYRVNPNLAFKAGAAINTSGNKKGSYNIGVNYEF
SEQ ID NO: 52 UspA2 American SP12-5 (686 amino acids)
MKTMKLLPLKIAVTSAMIIGLGAASTANAQATETFLPNLFDNDYTETTDPLYHGMILGNTAITQDTQYKF
YAENGNEVPDSLFFNKILHDQQLNGFKEGDTIIPLDENGKPVYKLDEITENGVKRKVYSVTTKTATRED
VEQSAYSRGIQGDIDDLYEANKENVNRLIEHGDKIFANEESVQYLNKEVQNNIENIHELAQQQDQHSS
DIKTLKKNVEEGLLELSGRLIAQKEDIAQNQTDIQDLATYNELQDQYAQKQTEAIDALNKASSENTQNIA
KNSNHIKTLENNIEEGLLELSGHLIDQKADLTKDIKALESNVEEGLLDLSGRLLDQKADIAKNQADIAQN
QTDIQDLAAYNELQDQYAQKQTEAIDALNKASSENTQNIEDLAAYNELQDAYAKQQTEAIDALNKASS
ENTQNIEDLAAYNELQDAYAKQQTEAIDALNKASSENTQNIAKNQADIANNINNIYELAQQQDQHSSDI
KTLAKASAANTDRIAKNKADADASFETLTKNQNTLIEKDKEHDKLITANKTAIDANKASADTKFAATADA
ITKNGNAITKNAKSITDLGTKVDGFDGRVTALDTKVNALDTKVNAFDGRITALDSKVENGMAAQAALSG
LFQPYSVGKFNATAALGGYGSKSAVAIGAGYRVNPNLAFKAGAAINTSGNKKGSYNIGVNYEF
SEQ ID NO: 53 UspA2 Swedish BCS (630 amino acids)
MKTMKLLPLKIAVTSAMIIGLGAASTANAQAKNDITLEDLPYLIKKIDQNELEADIGDITALEKYLALSQYG
NILALEELNKALEELDEDVGWNQNDIANLEDDVETLTKNQNALAEQGEAIKEDLQGLADFVEGQEGKIL
QNETSIKKNTQRNLVNGFEIEKNKDAIAKNNESIEDLYDFGHEVAESIGEIHAHNEAQNETLKGLITNSIE
NTNNITKNKADIQALENNVVEELFNLSGRLIDQKADIDNNINNIYELAQQQDQHSSDIKTLKKNVEEGLL
ELSGHLIDQKTDIAQNQANIQDLATYNELQDQYAQKQTEAIDALNKASSENTQNIEDLAAYNELQDAYA
KQQTEAIDALNKASSENTQNIEDLAAYNELQDAYAKQQTEAIDALNKASSENTQNIAKNQADIANNINNI
YELAQQQDQHSSDIKTLAKASAANTDRIAKNKADADASFETLTKNQNTLIEKDKEHDKLITANKTAIDAN
KASADTKFAATADAITKNGNAITKNAKSITDLGTKVDGFDSRVTALDTKVNAFDGRITALDSKVENGMA
AQAALSGLFQPYSVGKFNATAALGGYGSKSAVAIGAGYRVNPNLAFKAGAAINTSGNKKGSYNIGVN
YEF
SEQ ID NO: 54 UspA2 American 7169 (616 amino acids)
MKTMKLLPLKIAVTSALIVGLGAASTANAQAQDRSLEQIQDKLANLVEKIEQAKSQNGQSQKDINQYLL
LSQYANVLTMEELNNNVVKNSSSIETLDNDIAWLNDDLIDLDKEVGVLSRDIGSLHDDVAQNQADIKTL
KNNVVEELFNLSDRLIDQEADIAQNNESIEDLYDFGREVAESIGEIHAHNEAQNETLKDLITNSVKNTDN
ITKNKADIQALENDVGKELLNLSGRLIDQKADIDNNINHIYELAQQQDQHSSDIKTLKNNVEEGLLELSG
HLIDQKADLTKDIKALESNVEEGLLDLSGRLLDQKADIAQNQANIQDLAAYNELQDAYAKQQTEAIDAL
NKASSENTQNIEDLAAYNELQDAYAKQQTEAIDALNKASSENTQNIAKNQADIANNINNIYELAQQQDQ
HSSDIKTLAKASAANTDRIAKNKADADASFETLTKNQNTLIEKDKEHDKLITANKTAIDANKASADTKFA
ATADAITKNGNAITKNAKSITDLGTKVDGFDSRVTALDTKVNAFDGRITALDSKVENGMAAQAALSGLF
QPYSVGKFNATAALGGYGSKSAVAIGAGYRVNPNLAFKAGAAINTSGNKKGSYNIGVNYEF
SEQ ID NO: 55 UspA2 Finnish FIN2344 (614 amino acids)
MKTMKLLPLKIAVTSAMIIGLGATSTVNAQVVEQFFPNIFFNENHDELDDAYHNMILGDTAIVSNSQDNS
TQLKFYSNDEDSVPDSLLFSKLLHEQQLNGFKAGDTIIPLDKDGKPVYTKDTRTKDGKVETVYSVTTKI
ATQDDVEQSAYSRGIQGDIDDLYDINREVNEYLKATHDYNERQTEAIDALNKASSANTDRIDTAEERID
KNEYDIKALESNVGKDLLDLSGRLIAQKEDIDNNINHIYELAQQQDQHSSDIKTLKNNVEEGLLELSGHL
IDQKADLTKDIKTLESNVEEGLLDLSGRLIDQKADIAQNQANIQDLAAYNELQDQYAQKQTEAIDALNKA
SSENTQNIEDLAAYNELQDAYAKQQTEAIDALNKASSENTQNIAKNQADIANNINNIYELAQQQDQHSS
DIKTLAKVSAANTDRIAKNKADADASFETLTKNQNTLIEKDKEHDKLITANKTAIDANKASADTKFAATA
DAITKNGNAITKNAKSITDLGTKVDGFDGRVTALDTKVNAFDGRITALDSKVENGMAAQAALSGLFQPY
SVGKFNATAALGGYGSKSAVAIGAGYRVNPNLAFKAGAAINTSGNKKGSYNIGVNYEF
SEQ ID NO: 56 UspA2 American V1118 (679 amino acids)
MKTMKLPPLKIAVTSAMIIGLGAASTANAQTTETFLPNLFDNDYTETTDPLYHGMILGDTAITQDTQYKF
YAENGNEVPDSLFFNKILHDQLLNGFKAGDTIIPLDENGKPVYKLDERTENGVKRKVYSVTTKTATQAD
VEQSAYSRGIQGDIDDLYEANKENVNRLIEHGDKIFANEESVQYLNREVQNNIENIHELAQQQDQHSS
DIKTLKKNVEKDLLDLSGRLIAQKEDIAQNQTDIQDLATYNELQDQYAQKQTEAIDALNKASSENTQNIA
KNSNHIKTLENNIEECLLELSGHLIDQKADLTKDIKALESNVEEGLLDLSGRLIDQKADIAQNQANIQDLA
AYNELQDAYAKQQTEAIDALNKASSENTQNIEDLAAYNELQDAYAKQQTEAIDALNKASSENTQNIEDL
AAYN ELQDAYAKQQTEAIDALNKASSENTQNIAKNQADIANNINNIYELAQQQDQHSSDIKTLAKASAA
NTDRIAKNKADADASFETLTKNQNTLIEKDKEHDKLITANKTAIDANKASADTKFAATADAITKNGNAITK
NAKSITDLGTKVDGFDGRVTALDTKVNALDTKVNAFDGRITALDSKVENGMAAQAALSGLFQPYSVGK
FNATAALGGYGSKSAVAIGAGYRVNPNLAFKAGAAINTSGNKKGSYNIGVNYEF
SEQ ID NO: 57 UspA2 American V1145 (724 amino acids)
MKTMKLLPLKIAVTSALIVGLGAASTANAQETLEEVLESIKQINEQDLQDDIGYNSALDRYLVLSQYGNL
LIAKELNENVEKNSNSIAKNSNSIADLEADVGYLAENQNTLIEQNETINQELEGITHELESFIAYAHAQDQ
KNLVNEFEIEKNKDAIAKNNESIEDLYDFGHEVAESIGEIHAYTEEVNKTLENLITNSVKNTDNITKNKADI
QALESNVEKELLNLSGRLIDQKADIDNNINHIYELAQQQDQHSSDIKTLKKNVEEGLLELSGHLIDQKSD
IAQNQTDIQDLATYNELQDQYAQKQTEAIDALNKASSENTQNIEDLAAYNELQDAYAKQQTEAIDALNK
ASSENTQNIEDLAAYNELQDAYAKQQTEAIDALNKASSENTQNIEDLAAYNELQDAYAKQQTEAIDALN
KASSENTQNIEDLAAYNELQDAYAKQQTEAIDALNKASSENTQNIEDLAAYNELQDAYAKQQTEAIDAL
NKASSENTQNIAKNQADIANNINNIYELAQQQDQHSSDIKTLAKASAANTDRIAKNKADADASFETLTK
NQNTLIEKDKEHDKLITANKTAIDANKASADTKFAATADAITKNGNAITKNAKSITDLGTKVDGFDSRVT
ALDTKVNAFDGRITALDSKVENGMAAQAALSGLFQPYSVGKFNATAALGGYGSKSAVAIGAGYRVNP
NLAFKAGAAINTSGNKKGSYNIGVNYEF
SEQ ID NO: 58 UspA2 American V1156 (611 amino acids)
MKTMKLLPLKIAVTSALIVGLGAASTANAQAQARDRSLEDIQALIGNIDVDKIRSQKQKNPEIFQYLLLN
QLSNTLITDELNNNVIKNTNSIETLDNDIAVVLNDDLIDLDKEVGVLSRDIGSLHDDVAQNQADIKTLENN
VVEELFNLSDRLIDQEAEIAQNNESIEDLYDFGREVAESIGEIHAHNEAQNETLKDLITNSVKNTDNIDK
NKADIQALENNVEEGLLELSGHLIDQKADLTKDIKALESNVEEGLLDLSGRLLDQKADIAKNQADIAQN
QTDIQDLAAYNELQDQYAQKQTEAIDALNKASSENTQNIEDLAAYNELQDAYAKQQTEAIDALNKASS
ENTQNIEDLAAYNELQDAYAKQQTEAIDALNKASSENTQNIAKNQADIANNINNIYELAQQQDQHSSDI
KTLAKVSAANTDRIAKNKADADASFETLTKNQNTLIEKDKEHDKLITANKTAIDANKASADTKFAATADA
ITKNGNAITKNAKSITDLGTKVDGFDSRVTALDTKVNAFDGRITALDSKVENGMAAQAALSGLFQPYSV
GKFNATAALGGYGSKSAVAIGAGYRVNPNLAFKAGAAINTSGNKKGSYNIGVNYEF

1. A process for preparing a liquid composition comprising a Protein D polypeptide, wherein the Protein D polypeptide has at least 90% identity to SEQ ID NO: 2, wherein the process comprises mixing the Protein D polypeptide with sucrose and poloxamer.

2. The process for preparing a liquid composition comprising a Protein D polypeptide according to claim 1, wherein the process comprises mixing the Protein D polypeptide with sucrose and poloxamer prior to mixing the Protein D polypeptide with other antigens.

3. The process for preparing a liquid composition comprising a Protein D polypeptide according to claim 1, wherein the process comprises mixing the Protein D polypeptide with solution(s) comprising: (a) sucrose to a concentration of 5 to 20% (w/v), 10 to 20% (w/v), or 10 to 15% (w/v), and (b) poloxamer to a concentration of 0.1 to 1% (w/v), 0.5 to 1% (w/v), or 1% (w/v).

4. The process for preparing a liquid composition comprising a Protein D polypeptide according to claim 1, wherein the process comprises mixing the Protein D polypeptide with solution(s) comprising: (a) sucrose, (b) poloxamer and (c) a salt.

5. The process for preparing a liquid composition comprising a Protein D polypeptide according to claim 1, wherein the process comprises mixing the Protein D polypeptide with solution(s) comprising: (a) sucrose, (b) poloxamer, (c) a salt and (d) a buffer.

6. The process for preparing a liquid composition comprising a Protein D polypeptide according to claim 1, wherein the process comprises mixing the Protein D polypeptide with solution(s) comprising: (a) sucrose, (b) poloxamer (c) a salt and (d) a buffer, to reach a pH 6.4 to 7.7.

7. The process for preparing a liquid composition comprising a Protein D polypeptide according to claim 1, wherein the process comprises the steps of: (i) thawing the Protein D polypeptide, and (ii) mixing the Protein D polypeptide with sucrose and poloxamer.

8. The process for preparing a liquid composition comprising a Protein D polypeptide according to claim 1, subsequently comprising the step of filtration to obtain a liquid composition comprising the Protein D polypeptide in the filtrate.

9. The process for preparing a liquid composition comprising a Protein D polypeptide according to claim 1, subsequently comprising the step of storing the liquid composition comprising the Protein D polypeptide.

10. The process for preparing a liquid composition comprising a Protein D polypeptide according to claim 1, subsequently comprising the step of mixing the liquid composition comprising the Protein D polypeptide with other antigen(s).

11. The process for preparing a liquid composition comprising a Protein D polypeptide according to claim 10, wherein the other antigens comprise a PE-PilA fusion protein and an UspA2 polypeptide.

12. The process for preparing a liquid composition comprising a Protein D polypeptide according to claim 1, which reduces the formation of Protein D polypeptide visible particles as compared to a process without the addition of sucrose and poloxamer to the Protein D polypeptide composition.

13. The process for preparing a liquid composition comprising a Protein D polypeptide according to the process of claim 1, and subsequently freeze-drying the liquid composition comprising the Protein D polypeptide.

14. The liquid composition comprising a Protein D polypeptide, sucrose and poloxamer.

15. The liquid composition according to claim 14, wherein the Protein D polypeptide has at least 90% identity to SEQ ID NO: 2 and is in an amount of 0.025 to 20 mg/ml, 0.5 to 10 mg/ml, 0.5 to 1 mg/ml, or 1 mg/ml; wherein the sucrose is in an amount of 5 to 20% (w/v), 10 to 20% (w/v), or 10 to 15% (w/v); wherein the poloxamer is in an amount of 0.1 to 1% (w/v), 0.5 to 1% (w/v), or 1% (w/v); and further comprising a buffer and a salt.

16. The liquid composition according to claim 15, wherein the poloxamer is poloxamer 188, the salt is NaCl, and the buffer is phosphate buffer.

17. The process for preparing a liquid composition comprising a Protein D polypeptide according to claim 3, wherein the poloxamer is poloxamer 188.

18. The process for preparing a liquid composition comprising a Protein D polypeptide according to claim 4, wherein the poloxamer is poloxamer 188 and the salt is NaCl.

19. The process for preparing a liquid composition comprising a Protein D polypeptide according to claim 5, wherein the poloxamer is poloxamer 188, the salt is NaCl, and the buffer is phosphate buffer.

20. The process for preparing a liquid composition comprising a Protein D polypeptide according to claim 6, wherein the poloxamer is poloxamer 188, the salt is NaCl, the buffer is phosphate buffer, and the pH is about 6.8.


SEQUENCE LISTING (58 sequences)

SEQ ID NO: 1 - protein, 364 aa, H. influenzae
MKLKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIK

SEQ ID NO: 2 - protein, 348 aa, Artificial
  Protein D fragment with MDP tripeptide from NS1
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIK

SEQ ID NO: 3 - protein, 10 aa, H. influenzae
SSHSSNMANT

SEQ ID NO: 4 - protein, 160 aa, H. influenzae
MKKIILTLSLGLLTACSAQIQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKK

SEQ ID NO: 5 - protein, 141 aa, H. influenzae
IQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKK

SEQ ID NO: 6 - protein, 149 aa, H. influenzae
MKLTTQQTLKKGFTLIELMIVIAIIAILATIAIPSYQNYTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 7 - protein, 110 aa, H. influenzae
TKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 8 - protein, 275 aa, Artificial
  LVL735 (protein) (pelB sp)(ProtE aa 20-160) (GG)(PilA aa40-149)
MKYLLPTAAAGLLLLAAQPAMAIQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKKGGTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 9 - protein, 253 aa, Artificial
  PE-PilA fusion protein without signal peptide
IQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKKGGTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 10 - protein, 630 aa, Moraxalla catarrhalis
MKTMKLLPLKIAVTSAMIIGLGAASTANAQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 11 - protein, 520 aa, Artificial
  MC-001 (protein) - (M)(UspA2 amino acids 30 - 540)(ASHHHHHH)
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKASHHHHHH

SEQ ID NO: 12 - protein, 512 aa, Moraxalla catarrhalis
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTK

SEQ ID NO: 13 - protein, 513 aa, Artificial
  MC-003 (Protein) - (M)(UspA2 amino acids 30- 540)(H)
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKH

SEQ ID NO: 14 - protein, 514 aa, Artificial
  MC-004 (Protein) - (M)(UspA2 amino acids 30- 540)(HH)
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKHH

SEQ ID NO: 15 - protein, 499 aa, Artificial
  MC-005 (Protein) - (M)(UspA2 amino acids 30- 519)(ASHHHHHH)
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSASHHHHHH

SEQ ID NO: 16 - protein, 491 aa, Moraxalla catarrhalis
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKS

SEQ ID NO: 17 - protein, 544 aa, Artificial
  MC-007 (Protein) - (M)(UspA2 amino acids 30- 564)(ASHHHHHH)
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAAASHHHHHH

SEQ ID NO: 18 - protein, 538 aa, Artificial
  MC-008 (Protein) - (M)(UspA2 30-564)(HH)
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAAHH

SEQ ID NO: 19 - protein, 537 aa, Artificial
  MC-009 (Protein) - (M)(UspA2 31-564)(HH)
MAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAAHH

SEQ ID NO: 20 - protein, 536 aa, Moraxalla catarrhalis
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAA

SEQ ID NO: 21 - protein, 519 aa, Artificial
  MC-011 (Protein) - (M)(UspA2 amino acids 31- 540)(ASHHHHHH)
MAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKASHHHHHH

SEQ ID NO: 22 - protein, 613 aa, Moraxalla catarrhalis
MKTMKLLPLKIAVTSAMIIGLGAASTANAQSRDRSLEDIQDSISKLVQDDINTLKQDQQKMNKYLLLNQLANTLITDELNNNVIKNTNSIEALGDEIGWLENDIADLEEGVEELTKNQNTLIEKDEEHDRLIAQNQADIQTLENNVVEELFNLSGRLIDQEADIAKNNASIEELYDFDNEVAERIGEIHAYTEEVNKTLENLITNSVKNTDNIDKNKADIDNNINHIYELAQQQDQHSSDIKTLKNNVEEGLLELSGHLIDQKADLTKDIKALESNVEEGLLDLSGRLLDQKADLTKDIKALESNVEEGLLDLSGRLLDQKADIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTNRIATAELGIAENKKDAQIAKAQANANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 23 - protein, 644 aa, Moraxalla catarrhalis
MKTMKLLPLKIAVTSALIIGLGAASTANAQQQLQTETFLPNFLSNDNYDLTDPFYHNMILGDTALLDKQDGSQPQLKFYSNDKDSVPDSLLFSKLLHEQQLNGFKKGDTIIPLDKDGKPVYQVDYKLDGKGKKQKRRQVYSVTTKTATDDDVNSAYSRGILGKVDDLDDEMNFLNHDITSLYDVTANQQDAIKDLKKGVKGLNKELKELDKEVGVLSRDIGSLNDDVAQNNESIEDLYDFSQEVADSIGEIHAHNKAQNETLQDLITNSVENTNNITKNKADIQALENNVVEELFNLSGRLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 24 - protein, 591 aa, Moraxalla catarrhalis
MKTMKLLPLKIAVTSALIVGLGAASTANAQLVERFFPNIFLDKPLAKQHYHNVVVGDTSIVSDLQSNSDQLKFYSDDEGLVPDSLLFNKMLHEQLLNGFKEGDTIIPLDENGKPVYKVDYKLDGKEPRKVYSVTTKIATAEDVATSSYANGIQKDIDDLYDFDHQVTERLTQHGKTIYRNGERILANEESVQYLNKEVQNNIEHIYELAQQQDQHSSDIKTLESNVEKGLLELSGHLIDQKADLTKDIKTLESNVEEGLLDLSGRLIDQKADLTKDIKTLESNVEEGLLDLSGRLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTNRIATAELGIAENKKDAQIAKAQANANKTAIDENKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 25 - protein, 687 aa, Moraxalla catarrhalis
MKTMKLLPLKIAVTSAMIIGLGAASTANAQQQQQQQQQQQSRTEIFFPNIFFNENHDELDDAYHNIILGDTALLDKQDGSQPQLKFYSNDKDSVPDSLLFSKLLHEQQLNGFKKGDTIIPLDKDGKPVYQVDYKLDGKGKKQKRRQVYSVTTKTATDDDVNSAYSRGILGKVDDLDDEMNFLNHDITSLYDVTANQQDAIKGLKKGVKGLNKELKELDKEVGVLSRDIGSLNDDVAQNNESIEDLYDFSQEVADSIGEIHAHNKAQNETLQDLITNSVENTNNITKNKADIQALENNVVEELFNLSGRLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDAFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 26 - protein, 683 aa, Moraxalla catarrhalis
MKTMKLLPLKIAVTSAMIVGLGMASTANAQQQKSPKTETFLPNIFFNEYADDLDTLYHNMILGDTAITHDDQYKFYADDATEVPDSLFFNKILHDQLLYGFKEGDKIIPLDENGKPVYKLDKRLENGVQKTVYSVTTKTATADDVNSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 27 - protein, 684 aa, Moraxalla catarrhalis
MKTMKLLPLKIAVTSAMMVGLGMASTANAQQQKSPKTEIFLPNLFDNDNTELTDPLYHNMILGNTALLTQENQYKFYADDGNGVPDSLLFNKILHDQLLHGFKEGGTIIPLDENGKPVYKLDSIVEQGKTKTVYSVTTKTATADDVNSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 28 - protein, 684 aa, Moraxalla catarrhalis
MKTMKLLPLKIAVTSAMIIGLGAASTANAQQQQKTKTEVFLPNLFDNDYYDLTDPLYHSMILGDTATLFDQQDNSKSQLKFYSNDKDSVPDSLLFSKLLHEQQLNGFKAGDTIIPLDKDGKPVYTQDTRTKDGKVETVYSVTTKIATQDDVEQSAYSRGIQGDIDDLYDINREVNEYLKATHDYNERQTEAIDALNKASSANTDRIDTAEERIDKNEYDIKALESNVGKDLLDLSGRLIAQKEDIDNNINHIYELAQQQDQHSSDIKTLKNNVEEGLLELSGHLIDQKADLTKDIKTLENNIEEGLLELSGHLIDQKADLTKDIKTLENNIEEGLLELSGHLIDQKADIAQNQANIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 29 - protein, 684 aa, Moraxalla catarrhalis
MKTMKLLPLKIAVTSAMMVGLGMASTANAQQQKSPKTEIFLPNLFDNDNTELTDPLYHNMILGNTALLTQENQYKFYADDGNGVPDSLLFNKILHDQLLHGFKKGDTIIPLDENGKPVYKLDSIVEQGKTKTVYSVTTKTATADDVNSAYSRGIQGDIDDLYEANKENVNRLIEHGDKIFANEESVQYLNREVQNNIENIYELVQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 30 - protein, 574 aa, Moraxalla catarrhalis
MKTMKLLPLKIAVTSAMIIGLGAASTANAQLAEQFFPNIFSNHAPVKQHYHNVVVGDTSIVENLQDSDDTQLKFYSNDEYSVPDSLLFNKMLHEQQLNGFKKGDTIIPLDENGKPVYKVDYKLDGQEPRRVYSVTTKIATQDDVDNSPYSRGIQGDIDDLYEANKENVNRLIEHGDKIFANEESVQYLNKEVQNNIENIYELAQQQDQHSSDIKTLKKNVEEGLLELSGHLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDAFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 31 - protein, 678 aa, Moraxalla catarrhalis
MKTMKLLPLKIAVTSALIVGLGAASTANAQQQPQTETFFPNIFFNENHDALDDVYHNMILGDTAITQDNQYKFYADAISEVPDSLLFNKILHDQQLNGFKEGDTIIPLDENGKPVYKLDEKVENGVKKSVYSVTTKTATRADVEQSAYSRGIQGDIDDLYEANKENVNRLIEHGDKIFANEESVQYLNKEVQNNIENIHELAQQQDQHSSDIKTLKKNVEEGLLELSGHLIDQKADLTKDIKTLESNVEEGLLDLSGRLLDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDAFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 32 - protein, 678 aa, Moraxalla catarrhalis
MKTMKLLPLKIAVTSAMIVGLGAASTANAQQQQQPRTETFFPNIFFNENHDALDDVYHNMILGDTAITQDNQYKFYADAISEVPDSLLFNKILHDQQLNGFKEGDTIIPLDENGKPVYKLDEKVENGVKKSVYSVTTKTATRADVEQSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKTLENNIEEGLLELSGHLIDQKADLTKDIKALESNVEEGLLDLSGRLLDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDTNKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 33 - protein, 587 aa, Moraxalla catarrhalis
MKTMKLLPLKIAVTSALIVGLGAASTANAQLVERFFPNIFLDKPLAKQHYHNVVVGDTSIVSDLQSNSDQLKFYSDDEGLVPDSLLFNKMLHEQLLNGFKEGDTIIPLDENGKPVYKVDYKLDGKEPRKVYSVTTKIATAEDVATSSYANGIQKDIDDLYDFDHQVTERLTQHGKTIYRNGERILANEESVQYLNKEVQNNIEHIYELAQQQDQHSSDIKTLESNVEKGLLELSGHLIDQKADLTKDIKTLENNVEEGLLDLSGRLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDTNKASADTKFAATADAITKNGNAITKNAKSITDLGTKVDGFDSRVTALDTKVNALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 34 - protein, 678 aa, Moraxalla catarrhalis; identical to SEQ ID NO: 32

SEQ ID NO: 35 - protein, 616 aa, Moraxalla catarrhalis
MKTMKLLPLKIAVTSALIVGLGAASTANAQVRDKSLEDIEALLGKIDISKLEKEKKQQTELQKYLLLSQYANVLTMEELNKNVEKNTNSIEALGYEIGWLENDIADLEEGVEELTKNQNTLIEKDEEHDRLIAQNQADIKTLENNVVEELFNLSDRLIDQEADIAKNNASIEELYDFDNEVAERIGEIHAYTEEVNKTLEKLITNSVKNTDNIDKNKADIQALENNVEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 36 - protein, 676 aa, Moraxalla catarrhalis
MKTMKLLPLKIAVTSALIVGLGAASTANAQATETFLPNLFDNDYTETTDPLYHGMILGNTAITQDTQYKFYAENGNEVPDSLFFNKILHDQQLNGFKEGDTIIPLDENGKPVYKLDEITENGVKRKVYSVTTKTATREDVEQSAYSRGIQGDIDDLYEANKENVNRLIEHGDKIFANEESVQYLNKEVQNNIENIHELAQQQDQHSSDIKTLKKNVEEGLLELSGHLIDQKADLTKDIKALESNVEEGLLDLSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 37 - protein, 629 aa, Moraxalla catarrhalis
MKTMKLLPLKIAVTSALIVGLGAASTANAQNGTSTKLKNLKEYAQYLDNYAQYLDDDIDDLDKEVGELSQNIAKNQANIKDLNKKLSRDIDSLREDVYDNQYEIVNNQADIEKNQDDIKELENNVGKELLNLSGRLLDQKADIDNNINNIYELAQQQDQHSSDIKTLKKNVEEGLLELSGHLIDQKSDIAQNQTDIQDLATYNELQDQYAQKQTEAIDALNKASSENTQNIEDLAAYNELQDAYAKQQTEAIDALNKASSENTQNIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDAFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 38 - protein, 683 aa, Moraxalla catarrhalis
MKTMKLLPLKIAVTSAMIVGLGMASTANAQQQRSPKTETFLPNIFFNEYADDLDTLYHNMILGDTAITHDDQYKFYADDATEVPDSLFFNKILHDQLLYGFKEGDKIIPLDENGKPVYKLDKRLDNGVQKTVYSVTTKTATADDVNSAYSRGIQGDIDDLYEANKENVNRLIEHGDKIFANEESVQYLNKEVQNNIENIHELAQQQDQHSSDIKTLKKNVEEGLLELSGHLIDQKTDIAQNQTDIQDLATYNELQDQYAQKQTEAIDALNKASSENTQNIAKNSNRIKALENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 39 - protein, 700 aa, Moraxalla catarrhalis
MKTMKLLPLKIAVTSAMIVGLGAASTANAQAQSNRSLDQVQALLRGIDETKIKKEIQQSQQPELNKYLTFNQLANALNIEELNNNVQKNTQRLDSAATLYGDLSKTVPKSIKENKESIKENKESIKENKESIKENKESIKENKESIKENKESITTLTRKSFQNQVDIVRNNASIEDLYAYGQEVAKSIGEIHAYTEEVNKTLENLITNSVENTNNITKNKADIQALENNVVEELFNLSGRLIDQKADIDNNINNIYELAQQQDQHSSDIKTLKKNVEEGLLELSGHLIDQKADLTKDIKTLESNVEEGLLDLSGRLLDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTVIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 40 - protein, 676 aa, Moraxalla catarrhalis
MKTMKLLPLKIAVTSALIVGLGAASTANAQATETFLPNLFDNDYIETTDPLYHGMILGNTAITQDTQYKFYAENGNEVPDSLFFNKILHDQQLNGFKEGDTIIPLDENGKPVYKLDEITENGVKRKVYSVTTKTATREDVEQSAYSRGIQGDIDDLYEANKENVNRLIEHGDKIFANEESVQYLNKEVQNNIENIHELAQQQDQHSSDIKTLKKNVEEGLLELSGHLIDQKADLTKDIKTLENNVEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 41 - protein, 678 aa, Moraxalla catarrhalis
MKTMKLLPLKIAVTSAMIIGLGAASTANAQLAEQFFPNIFSNHAPVKQHYHNVVVGDTSIVENLQDSDDTQLKFYSNDEYSVPDSLLFNKMLHEQQLNGFKKGDTIIPLDENGKPVYKVDYKLDGQEPRRVYSVTTKIATQDDVDNSPYSRGIQGDIDDLYEANKENVNRLIEHGDKIFANEESVQYLNKEVQNNIENIYELAQQQDQHSSDIKTLKKNVEEGLLELSGRLIDQKADIAQNQANIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 42 - protein, 613 aa, Moraxalla catarrhalis; identical to SEQ ID NO: 22

SEQ ID NO: 43 - protein, 589 aa, Moraxalla catarrhalis
MKTMKLLPLKIAVTSALIVGLGAASTANAQATNKDITLEDVLKSIEEIDPYELRDYIEYPTAIERFLLLSQYGNTLTLEEFDNDIELLDQDVEDLEESVTELAKNQNSLIEQGEAIKEDLQGLADFVERQEDKILQNETSIKKNTQRNLVNGFEIEKNKDAIAKNNESIEDLYDFGHEVAKSIGEIHAHNEAQNETLKDLITNSVKNTDNITKNKADIQALESNVEKGLLELSGHLIDQKADIDNNINNIHELAQQQDQHSSDIKTLKKNVEEGLLELSGHLIDQKSDIAQNQANIQDLATYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 44 - protein, 684 aa, Moraxalla catarrhalis
MKTMKLLPLKIAVTSAMMVGLGMASTANAQQQKSPKTEIFLPNLFDNDNTELTDPLYHNMILGNTALLTQENQYKFYADDGNGVPDSLLFNKILHDQLLHGFKEGDTIIPLDENGKPVYKLDSIVEQGKTKTVYSVTTKTATADDVNSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 45 - protein, 650 aa, Moraxalla catarrhalis
MKTMKLLPLKIAVTSALIVGLGAASTANAQQQQKTKTEVFLPNLFYNDYIEETDLLYHNMILGDTAALVDRQNYSNSQLKFYSNDEESVPDSLLFSKMLNNQQLNGFKAGDIIIPVDANGQVIYQKDTRVEGGKTRTVLSVTTKIATQQDVDSAYSRGIQGKVNDLDDEMNFLNHDITSLYDVTANQQDDIKGLKKGVKDLKKGVKGLNKELKELDKEVGVLSRDIGSLNDDVAQNNESIEDLYDFSQEVADSIGEIHAHNKAQNETLQDLITNSVENTNNITKNKADIQALENNVVEELFNLSGRLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 46 - protein, 616 aa, Moraxalla catarrhalis
MKTMKLLPLKIAVTSALIVGLGAVSTTNAQAQSRSLDQIQTKLADLAGKIAAGKNGGGQNNQNNQNDINKYLFLSQYANILTMEELNNNVVKNSSSIETLETDFGWLENDVADLEDGVEELTKNQNTLIEKDEEHDRLIAQNQADIQTLENNVVEELFNLSDRLIDQKADIAKNQADIAQNNESIEELYDFDNEVAEKIGEIHAYTEEVNKTLQDLITNSVKNTDNIDKNKADIDNNINHIYELAQQQDQHSSDIKTLKNNVEEGLLELSGHLIDQKADLTKDIKTLENNVEEGLLDLSGRLIDQKADIAKNQADIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 47 - protein, 668 aa, Moraxalla catarrhalis
MKTMKLLPLKIAVTSALIVGLGTASTANAQVASPANQKIQQKIKKVRKELRQDIKSLRNDIDSNTADIGSLNDDVADNQDDILDNQADIAKNQDDIEKNQADIKELDKEVGVLSREIGSLNDDIADNYTDIIDNYTDIIDNQANIAKNQDDIEKNQADIKELDKEVGVLSREIGSLNDDVADNQDDIAKNQADIQTLENNVEEGLLELSGHLLDQKADIDNNINNIYELAQQQDQHSSDIKTLKKNVEEGLLELSGHLIDQKTDIAQNQANIQDLATYNELQDQYAQEQTEAIDALNKASSENTQNIAKNSNRIKALESNVEEGLLELSGHLIDQKADLTKDIKALESNVEEGLLELSGHLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKVSADTKFAATADAITKNGNAITKNAKSITDLGTKVDAFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 48 - protein, 674 aa, Moraxalla catarrhalis
MKTMKLLPLKIAVTSAMIVGLGATSTVNAQVVEQFFPNIFFNENHDELDDAYHNMILGDTAIVSNSQDNSTQLKFYSNDEDSVPDSLLFSKLLHEQQLNGFKAGDTIIPLDKDGKPVYTKDTRTKDGKVETVYSVTTKIATQDDVEQSAYSRGIQGDIDDLYDINREVNEYLKATHDYNERQTEAIDALNKASSANTDRIDTAEERIDKNEYDIKALESNVEEGLLELSGHLIDQKADLTKDIKALESNVEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 49 - protein, 613 aa, Moraxalla catarrhalis
MKTMKLLPLKIAVTSAMIIGLGAASTANAQSRDRSLEDIQDSISKLVQDDIDTLKQDQQKMNKYLLLNQLANTLITDELNNNVIKNTNSIEALGDEIGWLENDIADLEEGVEELTKNQNTLIEKDEEHDRLIAQNQADIQTLENNVVEELFNLSGRLIDQEADIAKNNASIEELYDFDNEVAERIGEIHAYTEEVNKTLENLITNSVKNTDNIDKNKADIDNNINHIYELAQQQDQHSSDIKTLKNNVEEGLLELSGHLIDQKADLTKDIKALESNVEEGLLDLSGRLLDQKADLTKDIKALESNVEEGLLDLSGRLLDQKADIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTNRIATAELGIAENKKDAQIAKAQANANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 50 - protein, 576 aa, Moraxalla catarrhalis
MKTMKLLPLKIAVTSAMIVGLGATSTVNAQVVEQFFPNIFFNENHDELDDAYHNMILGDTAIVSNSQDNSTQLKFYSNDEDSVPDSLLFSKLLHEQQLNGFKAGDTIIPLDKDGKPVYTKDTRTKDGKVETVYSVTTKIATQDDVEQSAYSRGIQGDIDDLYDINREVNEYLKATHDYNERQTEAIDALNKASSANTDRIDTAEERIDKNEYDIKALESNVEEGLLELSGHLIDQKADLTKDIKALESNVEEGLLELSGHLIDQKADLTKDIKALESNVEEGLLDLSGRLLDQKADIAKNQADIAQNQTDIQDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 51 - protein, 684 aa, Moraxalla catarrhalis; identical to SEQ ID NO: 44

SEQ ID NO: 52 - protein, 686 aa, Moraxalla catarrhalis
MKTMKLLPLKIAVTSAMIIGLGAASTANAQATETFLPNLFDNDYTETTDPLYHGMILGNTAITQDTQYKFYAENGNEVPDSLFFNKILHDQQLNGFKEGDTIIPLDENGKPVYKLDEITENGVKRKVYSVTTKTATREDVEQSAYSRGIQGDIDDLYEANKENVNRLIEHGDKIFANEESVQYLNKEVQNNIENIHELAQQQDQHSSDIKTLKKNVEEGLLELSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 53 - protein, 630 aa, Moraxalla catarrhalis; identical to SEQ ID NO: 10

SEQ ID NO: 54 - protein, 616 aa, Moraxalla catarrhalis
MKTMKLLPLKIAVTSALIVGLGAASTANAQAQDRSLEQIQDKLANLVEKIEQAKSQNGQSQKDINQYLLLSQYANVLTMEELNNNVVKNSSSIETLDNDIAWLNDDLIDLDKEVGVLSRDIGSLHDDVAQNQADIKTLKNNVVEELFNLSDRLIDQEADIAQNNESIEDLYDFGREVAESIGEIHAHNEAQNETLKDLITNSVKNTDNITKNKADIQALENDVGKELLNLSGRLIDQKADIDNNINHIYELAQQQDQHSSDIKTLKNNVEEGLLELSGHLIDQKADLTKDIKALESNVEEGLLDLSGRLLDQKADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 55 - protein, 614 aa, Moraxalla catarrhalis
MKTMKLLPLKIAVTSAMIIGLGATSTVNAQVVEQFFPNIFFNENHDELDDAYHNMILGDTAIVSNSQDNSTQLKFYSNDEDSVPDSLLFSKLLHEQQLNGFKAGDTIIPLDKDGKPVYTKDTRTKDGKVETVYSVTTKIATQDDVEQSAYSRGIQGDIDDLYDINREVNEYLKATHDYNERQTEAIDALNKASSANTDRIDTAEERIDKNEYDIKALESNVGKDLLDLSGRLIAQKEDIDNNINHIYELAQQQDQHSSDIKTLKNNVEEGLLELSGHLIDQKADLTKDIKTLESNVEEGLLDLSGRLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 56 - protein, 679 aa, Moraxalla catarrhalis
MKTMKLPPLKIAVTSAMIIGLGAASTANAQTTETFLPNLFDNDYTETTDPLYHGMILGDTAITQDTQYKFYAENGNEVPDSLFFNKILHDQLLNGFKAGDTIIPLDENGKPVYKLDERTENGVKRKVYSVTTKTATQADVEQSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEECLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 57 - protein, 724 aa, Moraxalla catarrhalis
MKTMKLLPLKIAVTSALIVGLGAASTANAQETLEEVLESIKQINEQDLQDDIGYNSALDRYLVLSQYGNLLIAKELNENVEKNSNSIAKNSNSIADLEADVGYLAENQNTLIEQNETINQELEGITHELESFIAYAHAQDQKNLVNEFEIEKNKDAIAKNNESIEDLYDFGHEVAESIGEIHAYTEEVNKTLENLITNSVKNTDNITKNKADIQALESNVEKELLNLSGRLIDQKADIDNNINHIYELAQQQDQHSSDIKTLKKNVEEGLLELSGHLIDQKSDIAQNQTDIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 58 - protein, 611 aa, Moraxalla catarrhalis
MKTMKLLPLKIAVTSALIVGLGAASTANAQAQARDRSLEDIQALIGNIDVDKIRSQKQKNPEIFQYLLLNQLSNTLITDELNNNVIKNTNSIETLDNDIAWLNDDLIDLDKEVGVLSRDIGSLHDDVAQNQADIKTLENNVVEELFNLSDRLIDQEAEIAQNNESIEDLYDFGREVAESIGEIHAHNEAQNETLKDLITNSVKNTDNIDKNKADIQALENNVEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF